(19) United States
(12) Patent Application Publication
(10) Pub. No.: US 2026/0284241 A1
(43) Pub. Date: Sep. 24, 2026

(54) DOSAGE REGIMEN FOR SHORT-LIVED RADIOISOTOPE

(71) Applicant: AdvanCell Pty Limited, Sydney (AU)

(72) Inventors: Simon PUTTICK, Sydney (AU); Andrew ADAMOVICH, Sydney (AU); Stephen ROSE, Sydney (AU); Thomas KRYZA, Sydney (AU); Feifei LIU, Sydney (AU)

(21) Appl. No.: 19/545,906

(22) Filed: Feb. 20, 2026

Related U.S. Application Data
(63) Continuation of application No. 19/507,046, filed on Jul. 31, 2024.

(30) Foreign Application Priority Data
Jul. 31, 2023 (AU) 2023902414

(51) Int. Cl. A61K 51/08; A61P 35/04

(52) U.S. Cl. CPC A61K 51/088; A61P 35/04; A61K 2121/00

(57) ABSTRACT

There is provided a method for treating cancer in a human subject in need of treatment thereof, comprising administering to the human subject a course of treatment of an effective amount of a radiopharmaceutical comprising a therapeutic radioisotope having a half-life of less than about 24 hours, wherein at least part of the course of treatment comprises two or more separately dosed administrations of the radiopharmaceutical, each separate dose of radiopharmaceutical being independently administered at a dosage interval of about or less than about 4 weeks.

### CROSS REFERENCE TO RELATED APPLICATIONS

This application is a continuation of U.S. patent application Ser. No. 19/507,046, filed Jan. 30, 2026, which is a U.S. National Stage filing under 35 U.S.C. § 371 of International PCT Application No. PCT/AU2024/050810, filed Jul. 31, 2024, which claims the benefit of priority to Australian Application No. 2023902414, titled “Dosage Regimen for Short-Lived Radioisotope,” filed Jul. 31, 2023, each of which is incorporated herein by reference in its entirety.

### FIELD

The present disclosure relates generally to methods of treating cancer in a subject in need of treatment thereof, comprising administering to the subject a course of treatment of an effective amount of a radiopharmaceutical comprising a therapeutic radioisotope having a half-life of less than about 24 hours, wherein at least part of the course of treatment comprises two or more separately dosed administrations of the radiopharmaceutical, each separate dose of radiopharmaceutical being independently administered at a dosage interval of about or less than about 4 weeks.

### BACKGROUND

A promising emerging method for the treatment cancer involves the administration of targeted radiopharmaceuticals i.e. therapeutics which are labelled with a radioisotope that exhibit increased selectivity toward the cancer cells so as to deliver a toxic level of radiation to the cancer cells whilst sparing normal healthy tissues. Typically, radiopharmaceuticals are conjugate molecules comprising a targeting moiety with high affinity for a particular molecular target (e.g. a cell surface receptor), a chelator moiety (capable of forming a complex with a radioisotope), and optionally a small molecule linker or peptide-based linkage between the aforementioned moieties to effect conjugation, which itself may also contain other functionality for the purposes of modulating the overall properties of the therapeutic.

Radiopharmaceuticals comprising iodine (¹³¹I), yttrium (⁹⁰Y), or lutetium (¹⁷⁷Lu), with half-lives of approximately 8.0, 2.7 and 6.7 days respectively, represent a current standard of care of radiopharmaceutical therapy for the treatment of various cancers, such as prostate cancer, and are typically administered 4 to 6 times over a full course of treatment, with each administration occurring once every 6 to 8 weeks. The length of such intervals between administrations throughout a course of treatment has primarily been considered necessary as a result of the long half-life of the radioisotope and associated higher toxicity, which mandates an extended period in which systemic effects in the subject may be monitored. For patients with a short life expectancy (e.g. less than 12 months), such a prolonged treatment regime—which may be as long as e.g. 9 months—is far from ideal.

In light of the above, there is a need to identify new methods of treatment with radiopharmaceuticals that offer more convenient or advantageous courses of administration, high tumour uptake and efficacy, and which exhibit reduced or more tolerable systemic toxicity, or at least provide the public with a useful alternative.

### BRIEF DESCRIPTION OF DRAWINGS

FIG. 1 depicts ²¹²Pb-ADVC001 treatment in a mouse model of prostate cancer.

FIG. 2 depicts ²¹²Pb-SSTR2 ligand treatment in a mouse model of SSTR2-positive small cell lung cancer.

FIG. 3 depicts representative ²¹²Pb SPECT/CT images showing rapid tumour uptake of ²¹²Pb-ADVC001 highly concordant with tumour burden delineated on the pretreatment ¹⁸F-DCFPyl PET/CT images.

### SUMMARY

It has been surprisingly found that radiopharmaceuticals comprising short-lived isotopes (e.g. having a half-life of less than about 24 hours) can be safely and effectively administered to treat cancer with considerably shorter dosing frequencies (e.g. about or less than about 4 weeks between doses) than have previously thought to be viable. Importantly, the shorter schedule does not result in significant additional systemic toxicity, and in some instances may offer improved efficacy.

In one aspect, there is provided a method for treating cancer in a subject in need of treatment thereof, comprising administering to the subject a course of treatment of an effective amount of a radiopharmaceutical comprising a therapeutic radioisotope having a half-life of less than about 24 hours, wherein at least part of the course of treatment comprises two or more separately dosed administrations of the radiopharmaceutical, each separate dose of radiopharmaceutical being independently administered at a dosage interval of about or less than about 4 weeks.

In a related aspect, there is provided a method for treating cancer in a subject in need of treatment thereof, comprising administering to the subject two or more separately dosed administrations of a radiopharmaceutical comprising a therapeutic radioisotope having a half-life of less than about 24 hours, each separate dose of radiopharmaceutical being independently administered at a dosage interval of about or less than about 4 weeks.

In one embodiment, each separate dose of radiopharmaceutical is independently administered at a dosage interval (in weeks) of between about 0.1 to about 4, preferably between about 1 to about 3, more preferably between about 1 to about 2.

In one embodiment, the at least part of the course of treatment comprises between 2 to 20 separately dosed administrations of the radiopharmaceutical, preferably between 3 to 10 separately dosed administrations of the radiopharmaceutical, more preferably between 4 to 6 separately dosed administrations of the radiopharmaceutical, even more preferably 4 separately dosed administrations of the radiopharmaceutical.

In one embodiment, each dose of the radiopharmaceutical is evenly distributed over the at least part of the course of treatment.

In one embodiment, the at least part of the course of treatment has a dosing frequency (Q) defined by the formula: Q=A^{W}×Z, wherein: A^{W} is a dosage interval (in weeks) of between about 0.1 and about 4; and Z is the number of administered doses of between 2 and 200. In one embodiment, A^{W} is 1 or 2 and Z is 4.

In one embodiment, the at least part of the course of treatment (in weeks) is about or less than 100, 80, 60, 40, 30, 16, 12, 10, 9, 8, 6, 4, 3, 2, 1, 0.5, 0.3, or 0.2.

In one embodiment, the radiopharmaceutical has an activity per dose of between about 1 MBq to 20000 MBq. In one embodiment, the radiopharmaceutical has an activity per dose of between about 60 MBq to 500 MBq.

In one embodiment, the radiopharmaceutical is a PSMA-targeting radiopharmaceutical. In one embodiment, the radiopharmaceutical is a SSTR2-targeting radiopharmaceutical.

In one embodiment, the therapeutic radioisotope having a half-life of less than about 24 hours comprises a therapeutic radioisotope for emitting one or more of alpha, beta, gamma, x-ray, and auger electron radiation in an amount effective to damage a cancer cell, preferably alpha radiation.

In one embodiment, the therapeutic radioisotope is selected from the group consisting of ⁶⁴Cu, ¹⁶⁵Dy, ¹⁸⁸Re, ²¹¹At, ²¹²Pb, ²¹²Bi, or ²¹³Bi.

In one embodiment, the therapeutic radioisotope is ²¹²Pb or ²¹²Bi, more preferably ²¹²Pb.

In one embodiment, each separate dose of the radiopharmaceutical is parenterally administrated.

In one embodiment, each separate dose of the radiopharmaceutical is independently intravenously, topically or intravesically administered.

In one embodiment, the cancer is a PSMA expressing cancer.

In one embodiment, the PSMA expressing cancer is prostate cancer, preferably metastatic prostate cancer, more preferably metastatic castrate-resistant prostate cancer (mCRPC).

In one embodiment, the at least part of the course of treatment provides a reduction in average tumour volume (in %) of at least about 5.

In another aspect, there is provided use of a therapeutic radioisotope having a half-life of less than about 24 hours for the manufacture of a medicament for the treatment of cancer in a subject, wherein medicament is formulated to be administered to the subject as two or more separately dosed administrations of the radiopharmaceutical, each separate dose of radiopharmaceutical being independently administered at a dosage interval of about or less than about 4 weeks.

Other aspects and embodiments relating to the present disclosure are described herein. It will be appreciated that each example, aspect and embodiment of the present disclosure described herein is to be applied mutatis mutandis to each and every other example, aspect or embodiment unless specifically stated otherwise. The present disclosure is not to be limited in scope by the specific examples described herein, which are intended for the purpose of exemplification only. Functionally-equivalent substituents, compositions, methods and processes are clearly within the scope of the disclosure as described herein.

### DETAILED DESCRIPTION

The present disclosure describes the following various non-limiting embodiments, which relate to a method for treating cancer in a subject in need of treatment thereof, comprising administering to the subject a course of treatment of an effective amount of a radiopharmaceutical comprising a therapeutic radioisotope having a half-life of less than about 24 hours, wherein at least part of the course of treatment comprises two or more separately dosed administrations of the radiopharmaceutical, each separate dose of radiopharmaceutical being independently administered at a dosage interval of about or less than about 4 weeks.

### General Terms

In the following description, reference is made to the accompanying drawings which form a part hereof, and which is shown, by way of illustration, several embodiments. It is understood that other embodiments may be utilized and structural changes may be made without departing from the scope of the present disclosure.

With regards to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims. Furthermore, unless otherwise required by context, singular terms shall include pluralities and plural terms shall include the singular.

All publications discussed and/or referenced herein are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, processes, and compositions, referred to or indicated in this specification, individually or collectively, and any and all combinations or any two or more of said steps or features.

The term “and/or”, e.g., “X and/or Y” shall be understood to mean either “X and Y” or “X or Y” and shall be taken to provide explicit support for both meanings or for either meaning.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

As used herein, the term “about”, unless stated to the contrary, typically refers to a range of up to +/−10% of the designated value, and includes smaller ranges therein, for example +/−5% or +/−1% of the designated value.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 4.5, 4.75, and 5, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Specific Terms

As used herein, “administer” or “administration” shall refer to the means by which the radiopharmaceutical is delivered to a subject's body. It will be appreciated that in some embodiments “administer” or “administration” may refer to delivery of a dose of the radiopharmaceutical, and in some embodiments to the multiplicity of delivered (or intended to be delivered) doses of the radiopharmaceutical according to a “course of treatment”, such that it may be said that the “course of treatment” is administered.

As used herein, unless otherwise indicated the term “half-life” refers to radioactive half-life, which is the interval of time required for one half of the atomic nuclei in a given radioactive sample to decay.

As used herein, the term “radiopharmaceutical” refers to a radioactive molecular entity comprising a targeting moiety (non-limiting examples of which include small molecules or biological molecules) that is able to target specific organs, tissues, cells, or proteins within the body and a therapeutic radioisotope. In some embodiments, the radiopharmaceutical further comprises a chelator moiety that is bonded (for example, via covalent and/or non-covalent bonds) to the targeting moiety, and which contains, binds, or complexes the therapeutic radioisotope. It will be understood that “radiopharmaceutical” also encompasses any pharmaceutically acceptable salt, solvate, or enantiomer thereof.

As used herein, the term “subject” refers to any organism susceptible to a disease or condition that requires therapy. For example, the subject can be a mammal, primate, livestock (e.g., sheep, cow, horse, pig), companion animal (e.g., dog, cat), or laboratory animal (e.g., mouse, rabbit, rat, guinea pig, hamster). In one example, the subject is a mammal. In one embodiment, the subject is human.

As used herein, the term “treating” or “treatment” includes alleviation of the symptoms associated with a specific disease or condition and reducing and/or eliminating said symptoms. For example, the term “treating a PSMA-expressing cancer” refers to alleviating the symptoms associated with a PSMA-expressing cancer and/or eliminating the symptoms associated with a PSMA expressing cancer, such as prostate cancer. In certain embodiments, the terms refer to minimizing the advancement or worsening of the disease, disorder, or condition resulting from the administration of a formulation of the invention to a patient with such a disease, disorder, or condition. The terms “treat,” “treating”, “treatment”, or the like, as used herein covers the treatment of a disease, disorder, or condition in a subject, e.g., a mammal, and includes at least one of: (i) inhibiting the disease, disorder, or condition, i.e., partially or completely halting its progression; (ii) relieving the disease, disorder, or condition, i.e. causing regression of symptoms of the disease, disorder, or condition, or ameliorating a symptom of the disease, disorder, or condition; and (iii) reversal or regression of the disease, disorder, or condition, preferably eliminating or curing of the disease, disorder, or condition. In some embodiments, the terms refer to the administration of a radiopharmaceutical, after the onset of symptoms of the particular disease, disorder, or condition. As is known in the art, adjustments for age, body weight, general health, sex, diet, time of administration, drug interaction and the severity of the condition may be necessary, and will be ascertainable with routine experimentation by one of ordinary skill in the art based on the invention described herein.

As used herein, the term “preventing” or “prevention” includes prophylaxis of the specific disorder or condition. For example, the term “preventing a PSMA-expressing cancer” refers to preventing the onset or duration of the symptoms associated with a PSMA expressing cancer, such as prostate cancer.

As would be understood by the person skilled in the art, a radiopharmaceutical can be administered in a therapeutically effective amount. The term “therapeutically effective amount”, as used herein, refers to a radiopharmaceutical, being administered in an amount sufficient to alleviate or prevent to some extent one or more of the symptoms of the disorder or condition being treated. The result can be the reduction and/or alleviation of the signs, symptoms, or causes of a disease or condition, or any other desired alteration of a biological system. For example, one result may be the reduction of one or more symptoms associated with e.g. a PSMA-expressing cancer, such as prostate cancer.

The term, “effective amount”, as used herein, refers to an amount of a radiopharmaceutical, effective to achieve a desired pharmacologic effect or therapeutic improvement without undue adverse side effects. By way of example only, therapeutically effective amounts may be determined by routine experimentation, including but not limited to a dose escalation clinical trial. The term “therapeutically effective amount” includes, for example, a prophylactically effective amount. In one embodiment, a prophylactically effective amount is an amount sufficient to prevent a cancer, e.g. PSMA-expressing cancer, or prostate cancer. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician. Thus, it is not always possible to specify an exact “effective amount”. However, an appropriate “effective amount” in any individual case may be determined by one of ordinary skill in the art using routine experimentation. Where more than one therapeutic agent is used in combination, a “therapeutically effective amount” of each therapeutic agent can refer to an amount of the therapeutic agent that would be therapeutically effective when used on its own, or may refer to an adjusted (e.g., reduced) amount that is therapeutically effective by virtue of its combination with one or more additional therapeutic agents.

The term “onset” of activity, as used herein, refers to the length of time to alleviate or prevent to some extent one or more of the symptoms of the disorder or condition being treated following the administration of the radiopharmaceutical. The term “duration” refers to the length of time that the therapeutic continues to be therapeutically effective, i.e., alleviate or prevent to some extent one or more of the symptoms of the disorder or condition being treated. The person skilled in the art would be aware that onset, peak, and duration of therapy may vary depending on factors such as the patient, the condition of the patient, and the route of administration.

The terms “pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, colors, lubricants, preservatives, stabilizers (including those against radiolytic degradation), scavengers (e.g. metal chelators such as diethylenetriaminepentaacetic acid (DTPA), ethylenediaminetetraacetic acid [EDTA]), wetting agents, emulsifiers, salts for influencing osmotic pressure, buffers, coloring, and/or aromatic substances and the like (collectively referred to herein as “excipients”) that do not deleteriously react with the active component(s) of the composition. Non-limiting examples of stabilisers against radiolytic degradation include gentisic acid (2,5-dihydroxybenzoic acid) and salts thereof, ascorbic acid (L-ascorbic acid, vitamin C) and salts thereof (e.g. sodium ascorbate), methionine, histidine, melatonin, ethanol, and Se-methionine. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport. In one example, the compound of Formula (1) is an acetate salt.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine. It will be understood that the present disclosure encompasses solvated forms, including hydrates, of the compounds of Formula (1) and salts thereof.

Those skilled in the art of organic and/or medicinal chemistry will appreciate that the compounds of Formula (1) and salts thereof may be present in amorphous form, or in a crystalline form. It will be understood that the present disclosure encompasses all forms and polymorphs of the compounds of Formula (1) and salts thereof.

As used herein, the term “stereoisomer” refers to compounds having the same molecular Formula and sequence of bonded atoms (i.e., atom connectivity), though differ in the three-dimensional orientations of their atoms in space. As used herein, the term “enantiomers” refers to two compounds that are stereoisomers in that they are non-superimposable mirror images of one another. Relevant stereocenters may be denoted with (R)- or (S)-configuration.

The compounds of the present disclosure may contain chiral (asymmetric) centers or the molecule as a whole may be chiral. The individual stereoisomers (enantiomers and diastereoisomers) and mixtures of these are within the scope of the present disclosure.

The term “halo” or “halogen” whether employed alone or in compound words such as haloalkyl, represents fluorine, chlorine, bromine or iodine. Further, when used in compound words such as haloalkyl, the alkyl may be partially halogenated or fully substituted with halogen atoms which may be independently the same or different. Examples of haloalkyl groups include fluoromethyl, chloromethyl, bromomethyl, iodomethyl, fluoropropyl, fluorobutyl, difluoromethyl difluoroethyl, trifluoromethyl and trifluoroethyl groups. Further examples of haloalkyl groups include —CF₃, —CCl₃, and —CH₂CF₃, —CF₂CF₃ and —CH₂CHFCl.

As used herein, the term “alkyl” whether used alone, or in compound words such as haloalkyl, cycloalkyl, alkylcycloalkyl, alkylcarbocyclyl, heteroalkyl, alkylheterocyclyl, alkylheteroaryl, alkylamide, alkylphosphonate and alkylaryl, represents straight chain (i.e. linear) or branched chain hydrocarbon groups. Examples of alkyl groups include methyl, ethyl, n-propyl, iso-propyl, n-butyl, t-butyl, i-butyl, sec-butyl, pentyl, hexyl, heptyl, octyl, nonyl and decyl groups. In one example, the alkyl group is of 1 to 20 carbon atoms (i.e. C₁₋₂₀alkyl). In another examples, the alkyl is a group of 1 to 10 carbon atoms (i.e. C₁₋₁₀alkyl). In another example, the alkyl group is of 1 to 6 carbon atoms (i.e. C₁₋₆alkyl).

As used herein, the term “heteroalkyl” represents straight chain (i.e. linear) or branched chain hydrocarbon groups which are analogous to an alkyl group, but in which one or more carbon atoms is/are replaced by one or more heteroatoms selected from nitrogen, sulfur, and oxygen.

As used herein, the term “alkenyl” represents straight (i.e. linear) or branched chain unsaturated hydrocarbon groups containing at least one carbon-carbon double bond. Examples of alkenyl groups include ethylene, propenyl, butenyl, pentenyl, hexenyl, heptenyl, octenyl, nonenyl and decenyl groups. In one example, the alkenyl group is of 2 to 20 carbon atoms (i.e. C₂₋₂₀alkenyl). In another example, the alkenyl is a group 2 to 10 carbon atoms (i.e. C₂₋₁₀alkenyl). In another example, the alkenyl group is of 2 to 6 carbon atoms (i.e. C₂₋₆alkenyl)

As used herein, the term “alkynyl” represents straight (i.e. linear) or branched chain unsaturated hydrocarbon groups containing at least one carbon-carbon triple bond. Examples of alkenyl groups include, ethynyl, propynyl, butynyl, pentynyl, hexynyl, heptynyl, octynyl, nonynyl and decynyl groups. In one example, the alkynyl group is of 2 to 20 carbon atoms (i.e. C₂₋₂₀alkynyl). In one example, the alkynyl group is of 2 to 10 carbon atoms (i.e. C₂₋₁₀alkynyl). In another examples, the alkynyl group is of 2 to 6 carbon atoms (i.e. C₂₋₆alkynyl).

As used herein, the term “haloalkyl” represents to an alkyl group having at least one halogen substituent, where “alkyl” and “halogen” are as described above. For example, the haloalkyl group may have at least one, two or three halogen substituents. Examples of haloalkyl groups include fluoromethyl, chloromethyl, bromomethyl, iodomethyl, fluoropropyl, fluorobutyl, difluoromethyl difluoroethyl, trifluoromethyl and trifluoroethyl groups. Further examples of haloalkyl groups include —CF₃, —CCl₃, and —CH₂CF₃, —CF₂CF₃ and —CH₂CHFCl. In one example, the haloalkyl group is of 1 to 20 carbon atoms (i.e. C₁₋₂₀haloalkyl). In one example, the haloalkyl group is of 1 to 10 carbon atoms (i.e. C₁₋₁₀haloalkyl). In another example, the haloalkyl group is of 1 to 6 carbon atoms (i.e. C₁₋₆haloalkyl).

As used herein, the terms “carbocyclyl” and “carbocycle” whether used alone, or in compound words such as alkylcarbocyclyl, represents a monocyclic or polycyclic ring system wherein the ring atoms are all carbon atoms, e.g., of about 3 to about 20 carbon atoms, and which may be aromatic, non-aromatic, saturated, or unsaturated, and may be substituted and/or contain fused rings. In one example, the carbocyclyl group is of 3 to 20 carbon atoms (i.e. C₃₋₂₀-membered carbocyclyl). In another example, the carbocyclyl group is of 3 to 10 carbon atoms (i.e. C₃₋₁₀-membered carbocyclyl). Examples of such groups include aryl groups such as phenyl, naphthyl, anthracenyl or fluorenyl, saturated groups such as cycloalkyl and cycloalkenyl groups e.g. cyclopropyl, cyclobutyl, cyclopentyl, cyclopentenyl, cyclohexyl, cyclohexenyl, cycloheptyl, cyclooctyl, cyclononyl, cyclodecyl groups, or fully or partially hydrogenated phenyl, naphthyl and fluorenyl. It will be appreciated that the polycyclic ring system includes bicyclic and tricyclic ring systems.

As used herein, the term “cycloalkyl” whether used alone, or in compound words such as alkylcycloalkyl, refers to a monocyclic or polycyclic carbocyclic ring system of varying sizes, e.g., from about 3 to about 20 carbon atoms, e.g., cyclopropyl, cyclobutyl, cyclopentyl, cyclopentenyl, cyclohexyl, cyclohexenyl, cycloheptyl, cyclooctyl, cyclononyl, cyclodecyl. It will be appreciated that the polycyclic ring system includes bicyclic and tricyclic ring systems.

As used herein, the term “heterocyclyl” whether used alone or in compound words such as alkylheterocyclyl, refers to a monocyclic or polycyclic ring system wherein the ring atoms are provided by at least two different elements, typically a combination of carbon and one or more of nitrogen, sulfur, and oxygen, and wherein the ring system may be aromatic such as a “heteroaryl” group, non-aromatic, saturated, or unsaturated, and may be substituted and/or contain fused rings. Heterocyclyl groups containing a suitable nitrogen atom include the corresponding N-oxides. In one example, the heterocyclyl group is of 3 to 20 atoms (i.e. 3-20-membered heterocyclyl). In another example, the heterocyclyl group is of 3 to 10 atoms (i.e. 3-10-membered heterocyclyl). The heteroatom may preferably be N, O or S. Examples of monocyclic non-aromatic heterocyclyl groups include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl. Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl, and benzoazepanyl. Examples of monocyclic aromatic heterocyclyl groups (also referred to as monocyclic heteroaryl groups) include furanyl, thienyl, pyrrolyl, oxazolyl, thiazolyl, imidazolyl, oxadiazolyl, thiadiazolyl, pyridyl (e.g. the radical derived from pyridine), triazolyl, triazinyl, pyridazyl, isothiazolyl, isoxazolyl, pyrazinyl, pyrazolyl, and pyrimidinyl. Examples of bicyclic aromatic heterocyclyl groups (also referred to as bicyclic heteroaryl groups) include quinoxalinyl, quinazolinul, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridyl, pyridopyrimidinyl, isoquinolinyl, and benzohydroxazole. It will be appreciated that the polycyclic ring system includes bicyclic and tricyclic ring systems.

As used herein, amino acids may be referred to by their full name, three letter code, or single letter code, which will be understood by the person skilled in the art.

As will be understood, an “aromatic” group means a cyclic group having 4m+2 π electrons, where m is an integer equal to or greater than 1. As used herein, “aromatic” is used interchangeably with “aryl” to refer to an aromatic group, regardless of the valency of aromatic group.

As used herein, the term “aryl” whether used alone, or in compound words such as alkylaryl, represents an monocyclic (e.g. phenyl) or polycyclic (e.g. naphthyl) aromatic carbocyclic ring system. In one example, the aryl group is of 3 to 20 carbon atoms (i.e., an aromatic 3-20 membered carbocyclyl). In another example, the aryl group is of 3 to 10 carbon atoms (i.e., an aromatic 3-10 membered carbocyclyl). Examples of aryl groups include, but are not limited to, phenyl, naphthyl, anthracenyl or fluorenyl. It will be appreciated that the polycyclic ring system includes bicyclic and tricyclic ring systems. Related to the term aryl, the term “aralkyl” as used herein refers to an alkyl group wherein a hydrogen atom is replaced by an aryl group as a substituent. Examples of alkylaryl groups include, but are not limited to, an optionally substituted benzyl (e.g. —CH₂-phenyl).

As used herein, the term “heteroaryl” whether used alone, or in compound words such as alkylheteroaryl, represents a monocyclic or polycyclic aromatic ring system wherein the ring atoms are provided by at least two different elements, typically a combination of carbon and one or more of nitrogen, sulfur, and oxygen, and may be substituted and/or contain fused rings. Heteroaryl groups containing a suitable nitrogen atom include the corresponding N-oxides. In one example, the heteroaryl group is of 3 to 20 atoms (i.e. 3-20-membered heteroaryl). In another example, the heteroaryl group is of 3 to 10 atoms (i.e. 3-10-membered heteroaryl). Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl. Examples of bicyclic heteroaryl groups include quinoxalinyl, quinazolinul, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridyl, pyridopyrimidinyl, isoquinolinyl, and benzohydroxazole. All regioisomers are contemplated, e.g. 2-pyridyl, 3-pyridyl and 4-pyridyl. It will be appreciated that the polycyclic ring system includes bicyclic and tricyclic ring systems.

As used herein, the term “divalent linking moiety” refers to any divalent group capable of linking, joining, bonding or attaching two chemical moieties.

“—C(═O)—” represents a carbonyl linker group.

“—C(═O)O—” represents an ester linking group.

“—C(═O)S—” represents a thioester linking group.

“—C(═O)NH—” or “—C(═O)NR—” represents an amide linker group.

“—S(═O)₂—” represents a sulfone linker group.

“—S(═O)NH—” or “—S(═O)NR—” represents a sulfinamide linker group.

“—S(═O)₂NH—” or “—S(═O)₂NR—” represents a sulfonamide linker group.

“—OS(═O)₂—” represents a sulfonate ester linker group.

“—O—” represents an ether linker group.

“—NH—” or “—NR—” represents an amine linker group.

“—S—” represents a sulfide linker.

“—NHC(═S)NH—” or “—N(R)C(═S)N(R)—” represents a thiourea linker group.

“—NHC(═O)NH—” or “—N(R)C(═O)N(R)—” represents a urea linking group.

“—(CH₂)ₘ—” represents an alkylene linking group, including an alkylene bridge, having a defined number (“m”) of methylene (—CH₂—) units.

Unless otherwise stated or structurally depicted, it will be appreciated that the orientation of the linker groups described above and herein within the compound of Formula (1) are undefined. That is, the linker groups may be attached at either side within the compound of Formula (1).

As used herein, the term “saturated” refers to a group where all available valence bonds of the backbone atoms are attached to other atoms. Representative examples of saturated groups include, but are not limited to, butyl, cyclohexyl, and piperidine.

As used herein, the term “unsaturated” refers to a group where at least one valence bond of two adjacent backbone atoms is not attached to other atoms. Representative examples include, but are not limited to, alkenes (e.g., —CH₂CH═CH), phenyl, and pyrrole.

As used herein, the term “optionally substituted” means that a functional group is either substituted or unsubstituted, at any available position. As used herein, the term “substituted” refers to a group having one or more hydrogens or other atoms removed from a carbon or suitable heteroatom and replaced with a further group (i.e., substituent). As used herein, the term “unsubstituted” refers to a group that does not have any further groups attached thereto or substituted therefore.

### Methods of Treating Cancer

The present disclosure provides for a method for treating cancer in a subject in need of treatment thereof. The method comprises administering to the subject a course of treatment of an effective amount of a radiopharmaceutical comprising a therapeutic radioisotope having a half-life of less than about 24 hours, wherein at least part of the course of treatment comprises two or more separately dosed administrations of the radiopharmaceutical. Each separate dose of radiopharmaceutical can be independently administered at a dosage interval of about or less than about 4 weeks. Related to the method, the present disclosure also provides use of a therapeutic radioisotope having a half-life of less than about 24 hours for the manufacture of a medicament for the treatment of cancer in a subject, wherein medicament is formulated to be administered to the subject as two or more separately dosed administrations of the radiopharmaceutical, each separate dose of radiopharmaceutical being independently administered at a dosage interval of about or less than about 4 weeks. The embodiments and examples described herein in relation to the method of treatment equally apply to the use, and vice versa.

### Course of Treatment

The course of treatment may be carried out for as long a period as necessary. The course of treatment may comprise one or more parts. The course of treatment may, in some embodiments, comprise a single course of treatment that is definable according to a uniform dosing frequency and/or dosage. The period in which a part of a course of treatment is carried out may be of a duration independent to that of any one of, or all other, periods. It will be understood that a part of a course of treatment is identifiable from the other parts of the course of treatment that are immediately subsequent or preceding to that part, by reference to the distinctive dosage regimes that are carried out within each of, and characterises, those parts. The treating physician will know how to increase, decrease, or interrupt treatment based on patient response. The course of treatment, or part of a course of treatment, may be repeated as required.

In some embodiments, the at least part of the course of treatment (in weeks) is greater than about 0.2, 0.3, 0.4, 0.5, 0.6, 0.7, 0.8, 0.9, 1.0, 2, 3, 4, 5, 6, 7, 8, 9, 10, 12, 14, 16, 18, 20, 25, 30, 35, 40, 45, 50, 55, 60, 65, 70, 75, 80, 85, 90, 95, or 100. In some embodiments, the at least part of the course of treatment (in weeks) is less than about 100, 95, 90, 85, 80, 75, 70, 65, 60, 55, 50, 45, 40, 35, 30, 25, 20, 18, 16, 14, 12, 10, 9, 8, 7, 6, 5, 4, 3, 2, 1.0, 0.9, 0.8, 0.7, 0.6, 0.5, 0.4, 0.3, or 0.2. In some embodiments, the at least part of the course of treatment (in weeks) is in a range provided by any two of the previously described upper and/or lower amounts, for example between 0.2 and 100, 1 and 50, or 2 and 25.

### Dose-Frequency

It will be understood that the at least part of the course of treatment comprises two or more separately dosed administrations of the radiopharmaceutical (i.e. comprises multiple separately dosed administrations of the radiopharmaceutical).

In some embodiments, the separately dosed administrations of the radiopharmaceutical are independently administered at a dosage interval of (in weeks) greater than about 0.1, 0.2, 0.3, 0.4, 0.5, 0.6, 0.7, 0.8, 0.9, 1.0, 1.5, 2.0, 2.5, 3.0, 3.5, or 4.0. In some embodiments, the separately dosed administrations of the radiopharmaceutical are independently administered at a dosage interval (in weeks) less than about 0.1, 0.2, 0.3, 0.4, 0.5, 0.6, 0.7, 0.8, 0.9, 1.0, 1.5, 2.0, 2.5, 3.0, 3.5, or 4.0. In some embodiments, the separately dosed administrations of the radiopharmaceutical are independently administered at a dosage interval in a range provided by any two of the previously described upper and/or lower amounts, for example, the separately dosed administrations of the radiopharmaceutical are independently administered at a dosage interval (in weeks) between about 0.1 and about 4, about 1 and about 4, about 1 and about 3, or about 1 and about 2.

In some embodiments, the separately dosed administrations of the radiopharmaceutical are independently administered at a dosage interval of (in days) about or greater than about 1, 2, 3, 4, 5, 6, 7, 6, 8, 9, 10, 11, 12, 13, or 14. In some embodiments the dosage interval (in weeks) is about or less than about 14, 13, 12, 11, 10, 9, 8, 7, 6, 5, 4, 3, 2, or 1. In some embodiments, the dosage interval is in a range provided by any two of the previously described upper and/or lower amounts, for example, the dosage interval (in days) is between about 1 and about 14, about 1 and about 7, or about 7 and about 14.

In some embodiments, the number of separately administered doses of the radiopharmaceutical is greater than or equal to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200. In some embodiments, the number of separately administered doses of the radiopharmaceutical is between 1 and 200, 1 and 100, 2 and 20, 8 and 20, 8 and 18, 8 and 16, 8 and 14, 6 and 20, 6 and 18, 6 and 14, 6 and 12, 3 and 10, or 4 and 6.

The person skilled in the art will appreciate that the at least part of the course of treatment may be described according to standard dosing frequency nomenclature, which encompasses both the dosage interval for each separately administered dose and overall number of doses for the at least part of the course of treatment. Accordingly, in some embodiments, the at least part of the course of treatment has a dosing frequency (Q) defined by the formula:

$$Q = {A}^{W} \times Z$$

wherein:
A″ is a dosage interval (in weeks); and
Z is the number of administered doses.

In some embodiments, the dosage interval A^{W} (in weeks) is greater than about 0.1, 0.2, 0.3, 0.4, 0.5, 0.6, 0.7, 0.8, 0.9, 1.0, 1.5, 2.0, 2.5, 3.0, 3.5, or 4.0. In some embodiments the dosage interval A^{W} (in weeks) is less than about 0.1, 0.2, 0.3, 0.4, 0.5, 0.6, 0.7, 0.8, 0.9, 1.0, 1.5, 2.0, 2.5, 3.0, 3.5, or 4.0. In some embodiments, the dosage interval A^{W} is in a range provided by any two of the previously described upper and/or lower amounts, for example, the dosage interval A^{W} (in weeks) is between about 0.1 and about 4, about 1 and about 4, about 1 and about 3, or about 1 and about 2.

In some embodiments, the dosage interval A^{D} (in days) may be used instead of A^{w}. In some embodiments, the dosage interval in A^{D} (in days) is about or greater than about 1, 2, 3, 4, 5, 6, 7, 6, 8, 9, 10, 11, 12, 13, 14, 15, 16, 17, 18, 19, 20, or 21. In some embodiments the dosage interval A^{D} (in days) is about or less than about 21, 20, 19, 18, 17, 16, 15, 14, 13, 12, 11, 10, 9, 8, 7, 6, 5, 4, 3, 2, or 1. In some embodiments, the dosage interval A^{D} (in days) is in a range provided by any two of the previously described upper and/or lower amounts, for example, the dosage interval A^{D} (in days) is between about 1 and about 21, about 1 and about 14, about 2 and about 10, about 1 and about 7, or about 7 and about 14. It will be appreciated that where the dosage interval A^{D} refers to days, that the formula may be written as Q=A^{D}×Z.

In some embodiments, the number of administered doses Z is greater than 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200. In some embodiments the number of administered doses Z is less than 200, 190, 180, 170, 160, 150, 140, 130, 120, 110, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, or 2. In some embodiments, the number of administered doses Z is in a range provided by any two of the previously described upper and/or lower amounts, for example, the number of administered doses Z is between 1 and 200, 2 and 200, 1 and 100, 2 and 20, 8 and 20, 8 and 18, 8 and 16, 8 and 14, 6 and 20, 6 and 18, 6 and 14, 6 and 12, 3 and 10, or 4 and 6.

In some embodiments, the dosage interval A^{W} and the number of administered doses Z may be independently selected from any of the previously described amounts and/or ranges, for example, in some embodiments the dosage interval A^{W} is between 1 and 3 weeks, and/or the number of administered doses Z is between 6 and 20 (i.e. Q1-3W×6-20). In another example, the dosage interval A^{W} is 0.33 weeks, and the number of administered doses Z is 3 (i.e. Q0.33W×3). In another example, A^{W} is 0.33, and Z is 6 (i.e. Q0.33W×6). In another example, A^{W} is 1, and Z is 4 (i.e. Q1W×4). In another example, A^{W} is 1, and Z is 6 (i.e. Q1W×6). In another example, A^{W} is 2, and Z is 4 (i.e. Q2W×4). In another example, A is 2, and Z is 6 (i.e. Q2W×6). In another example, A^{W} is 3, and Z is 4 (i.e. Q3W×4). In another example, A^{W} is 3, and Z is 6 (i.e. Q3W×6). It will be understood that the present disclosure encompasses all possible combinations of dosage interval A^{W} or A^{D} with number of administered doses Z, wherein the dosage interval A^{W} or A^{D} and the number of administered doses Z may be selected from any such disclosed herein.

In another example, the dosage interval A^{D} (in days) is 1, 2, 3, 4, 5, 6, 7, 8, 9, 10, 11, 12, 13, or 14, or any range between any two integers thereof, and the number of administered doses Z is between 6 and 20. In another example, the dosage interval A^{D} (in days) is 2, 3, 4, 5, 6, 7, 8, 9, or 10, or any range between any two integers thereof, and the number of administered doses Z is between 6 and 20.

In another example, the dosage interval A^{W} (in weeks) is 1, 2, 3 or 4, and the number of administered doses Z is between 6 and 20. In another example, the dosage interval A^{W} (in weeks) is 1, 2, or 3, and the number of administered doses Z is between 6 and 20. In another example, the dosage interval A^{W} (in weeks) is 1 or 2, and the number of administered doses Z is between 6 and 20. In another example, the dosage interval A^{W} (in weeks) is 1, and the number of administered doses Z is between 6 and 20. In another example, the dosage interval A^{W} (in weeks) is 2, and the number of administered doses Z is between 6 and 20. In another example, the dosage interval A^{W} (in weeks) is 3, and the number of administered doses Z is between 6 and 20.

In some embodiments, the at least part of the course of treatment has a dosing frequency selected from the group consisting of: Q1W×6, Q1W×7, Q1W×8, Q1W×9, Q1W×10, Q1W×11, Q1W×12, Q1W×13, Q1W×14, Q1W×15, Q1W×16, Q1W×17, Q1W×18, Q1W×19, Q1W×20, Q2W×6, Q2W×7, Q2W×8, Q2W×9, Q2W×10, Q2W×11, Q2W×12, Q2W×13, Q2W×14, Q2W×15, Q2W×16, Q2W×17, Q2W×18, Q2W×19, Q2W×20, Q3W×6, Q3W×7, Q3W×8, Q3W×9, Q3W×10, Q3W×11, Q3W×12, Q3W×13, Q3W×14, Q3W×15, Q3W×16, Q3W×17, Q3W×18, Q3W×19, Q3W×20, Q4W×6, Q4W×7, Q4W×8, Q4W×9, Q4W×10, Q4W×11, Q4W×12, Q4W×13, Q4W×14, Q4W×15, Q4W×16, Q4W×17, Q4W×18, Q4W×19 and Q4W×20. In some embodiments, the at least part of the course of treatment has a dosing frequency selected from the group consisting of: Q1W×6, Q1W×7, Q1W×8, Q1W×9, Q1W×10, Q1W×11, Q1W×12, Q1W×13, Q1W×14, Q1W×15, Q1W×16, Q1W×17, Q1W×18, Q1W×19, Q1W×20. In some embodiments, the at least part of the course of treatment has a dosing frequency selected from the group consisting of: Q2W×6, Q2W×7, Q2W×8, Q2W×9, Q2W×10, Q2W×11, Q2W×12, Q2W×13, Q2W×14, Q2W×15, Q2W×16, Q2W×17, Q2W×18, Q2W×19 or Q2W×20. In some embodiments, the at least part of the course of treatment has a dosing frequency selected from the group consisting of: Q3W×6, Q3W×7, Q3W×8, Q3W×9, Q3W×10, Q3W×11, Q3W×12, Q3W×13, Q3W×14, Q3W×15, Q3W×16, Q3W×17, Q3W×18, Q3W×19 or Q3W×20.

In some embodiments, the at least part of the course of treatment has a dosing frequency of Q0.33W×3, Q0.33×6, Q1W×4, Q1W×6, Q2W×4, Q2W×6, Q3W×4, Q3W×6.

In some embodiments, the at least part of the course of treatment has a dosing frequency of Q0.33W×3, Q0.33×6, Q2W×4, Q2W×6, Q3W×4, Q3W×6.

In one embodiment, the at least part of the course of treatment comprises administering the radiopharmaceutical once per week. In some embodiments, the at least part of the course of treatment comprises administering the radiopharmaceutical once per week for up to 24, 18, 12, 9, 6, 4, 2 or 1 months.

In some embodiments, each dose of the radiopharmaceutical is evenly distributed over the at least part of the course of treatment. It will be understood that evenly distributed doses of the radiopharmaceutical are doses that have been administered to the subject with a common dosage interval (A) which may be described according to any aspect, embodiment, or example provided herein. It will be appreciated that for a dosage interval to be considered common across the at least part of the course of treatment, the actual interval between doses may vary by up to +/−1 week from the stated dosage interval defining the course of treatment (i.e. dosage interval A), as well as smaller ranges therein, for example +/−1, 2, 3, 4, 5, or 6 days of the stated dosage interval.

### Dose-Activity

The amount of the radiopharmaceutical that is administered to a subject depends on several physiological factors. These factors are known by the physician, including the nature of therapy to be carried out, tissue to be targeted for therapy and the body weight and medical history of the subject to be treated using the radiopharmaceutical. The activity of the radiopharmaceutical dose will principally depend upon the therapeutic radioisotope comprised by the radiopharmaceutical. It will be appreciated that a dose of the radiopharmaceutical may be described in terms of the total activity of that dose. It will be understood that activity per dose refers to the activity of a unitary dose, and is independent of the body weight of the subject to which it is administered.

Dosages may be varied depending upon the requirements of the patient and the radiopharmaceutical being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the subject's disease state.

In some embodiments, the radiopharmaceutical has an activity per dose (in MBq) greater than about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 2000, 5000, 10000, 15000, 20000. In some embodiments, the radiopharmaceutical has an activity per dose (in MBq) less than about 20000, 15000, 10000, 5000, 2000, 1000, 990, 980, 970, 960, 950, 940, 930, 920, 910, 900, 890, 880, 870, 860, 850, 840, 830, 820, 810, 800, 790, 780, 770, 760, 750, 740, 730, 720, 710, 700, 690, 680, 670, 660, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, 10, or 1. In some embodiments, the radiopharmaceutical has an activity per dose (in MBq) in a range provided by any two of the previously described upper and/or lower amounts, for example, the radiopharmaceutical has an activity per dose (in MBq) between about 1 to 20000, 1 to 1000, 1 to 600, 10 to 300, 20 to 500, 20 to 200, 60 to 500, 60 to 200, 100 to 200, or 120 to 200. Bq (becquerel) refers to an SI derived unit of radioactivity that is commonly understood by a person skilled in the art. One becquerel is defined as the amount of activity of a radioactive material when one nucleus decays per second.

If administered intravenously, an infusion of the radiopharmaceutical over a period of time may be used, and may include, for example, a dose escalation.

### Dose—Administration

It will be understood that “administer” or “administration” as used herein shall refer to the means by which the radiopharmaceutical is delivered to a subject's body. It will be appreciated that administration may refer to delivery of a dose of the radiopharmaceutical, or the multiplicity of delivered (or intended to be delivered) doses of the radiopharmaceutical according to a “course of treatment”, such that it may be said that the “course of treatment” is administered.

Each separate dose of the radiopharmaceutical may be administered via any known method suitable for delivery of the radiopharmaceutical. Specific modes of administration include, without limitation, inhalation, parenteral (including intravesical, intraperitoneal, intraarterial, intravenous, intranasal, intradermal, transdermal, subcutaneous, or intramuscular injection), topical, or oral. Also contemplated is the instillation of a drug in the body of the patient in a controlled formulation, with systemic or local release of the drug to occur at a later time. For example, the drug may be localized in a depot for controlled release to the circulation, or for release to a local site of tumour growth. Administration may be carried out by injection and/or delivery. The radiopharmaceutical may also be administered directly to the target site, e.g., by biolistic delivery to an external or internal target site.

In some embodiments, each separate dose of the radiopharmaceutical is parenterally administered. In some embodiments, each separate dose of the radiopharmaceutical is independently intravenously, topically or intravesically administered. In some embodiments, each separate dose of the radiopharmaceutical is independently intravenously administered. It some embodiments, all separate doses of the radiopharmaceutical are delivered according to the same mode of administration. In some embodiments, all separately dosed administrations of the radiopharmaceutical administered via the same mode of administration.

The mode of administration will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently.

In some embodiments, the patient being administered a radiopharmaceutical may remain radioactive following administration (e.g. have levels of radioisotope at or above the threshold considered to be radioactive by a medical practitioner) for between about 1 hours to about 96 hours. In some embodiments, the patient being administered a radiopharmaceutical may remain radioactive following administration (in hours) for at least about 1, 2, 3, 4, 5, 6, 8, 10, 12, 24, 48, 72, 96, or 120. In some embodiments, the patient being administered a radiopharmaceutical may remain radioactive following administration (in hours) for at least about 120, 96, 72, 48, 24, 12, 10, 8, 6, 5, 4, 3, 2, or 1. The patient may remain radioactive for a period of time in a range provided by any two of these upper and/or lower values, for example between about 1 to 24 hours, or between about 1 to 12 hours.

### Dose—Formulation

Whilst the radiopharmaceutical may in some embodiments be administered to a patient in need thereof alone, it is more typically administered as part of a pharmaceutical composition or formulation.

A pharmaceutical composition comprising a radiopharmaceutical may further comprise one or more pharmaceutically acceptable diluents, carriers, stabilisers (including those against radiolytic degradation), or excipients (collectively referred to herein as “excipients”). Examples of pharmaceutical compositions include those suitable for parenteral administration, including those suitable for intravesical, intraperitoneal, intraarterial, intravenous, intranasal, intravesical, intradermal, transdermal, subcutaneous, or intramuscular administration. Other examples of pharmaceutical compositions include those suitable for oral, inhalation, rectal, intraperitoneal and topical administration.

The excipient(s) must be pharmaceutically acceptable in the sense of being compatible with the other ingredients of the formulation and not unduly deleterious to the recipient thereof.

The pharmaceutically acceptable carrier may be an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the subject's needs.

Radioactive decay of the radionuclide occurs throughout manufacturing and storage of the radiopharmaceutical, releasing high energy emissions which may induce the cleavage of the chemical bonds within the radiopharmaceutical. This is often referred to as radiolysis or radiolytic degradation, and may lead to a decrease in therapeutic efficacy. One method for reducing radiolytic degradation, is to compose the radiopharmaceutical with one or more stabilisers against radiolytic degradation. Non-limiting examples of stabilisers against radiolytic degradation include gentisic acid (2,5-dihydroxybenzoic acid) and salts thereof, ascorbic acid (L-ascorbic acid, vitamin C) and salts thereof (e.g. sodium ascorbate), methionine, histidine, melatonin, ethanol, and Se-methionine.

In some embodiments, the radiopharmaceutical is formulated for parenteral delivery. For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, in one embodiment, the composition may be a sterile, lyophilized, crystalized or amorphous composition that is suitable for reconstitution in an aqueous vehicle prior to injection. In one embodiment, a composition suitable for parenteral administration conveniently comprises a sterile aqueous preparation of a radiopharmaceutical, which may for example be formulated to be isotonic with the blood of the recipient.

Sterile injectable solutions can be prepared by incorporating the radiopharmaceutical in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

In some embodiments, the radiopharmaceutical is formulated for oral delivery. Compositions for oral delivery may, for example, be in the form of tablets, lozenges, aqueous or oily suspensions, granules, powders, emulsions, capsules, syrups, or elixirs. Orally administered compositions may contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in a tablet or pill form, the compositions may be coated to delay disintegration and absorption in the gastrointestinal tract thereby providing a sustained action over an extended period of time. Oral compositions can include standard vehicles such as mannitol, lactose, starch, magnesium stearate, sodium saccharine, cellulose, magnesium carbonate, etc. Such vehicles are preferably of pharmaceutical grade. The oral compositions described herein may contain from about 1% to about 95% of a radiopharmaceutical weight. Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such compositions is such that a suitable dosage can be obtained.

The radiopharmaceutical may conveniently be presented in unit dosage form and may be prepared by any of the methods well known in the art of pharmacy. All methods include the step of bringing the radiopharmaceutical or a pharmaceutically acceptable salt, solvate or prodrug thereof, into association with a carrier that constitutes one or more accessory ingredients.

### Dose—Extrapolation

The person skilled in the art will appreciate that, in the normal development of any therapeutic, including a radiopharmaceutical, that human dosage regimens having suitable efficacy, toxicity and pharmacokinetic/dynamic profiles may be predicted by extrapolating from experiments (e.g. biodistribution) performed on laboratory animals (e.g. rats or mice). The person skilled in the art will be aware of the relevant factors that justify the scaling or adjustment of a parameter of a dosing regimen in the course of predicting a suitable translation or extrapolation of that dosing regimen from a laboratory animal to a human. Without intending to limit approaches that may be suitable for animal-to-human dosimetry extrapolation, various computational methods may be adopted, e.g. application of organ time-integrated activity coefficients, relative mass scaling, metabolic time scaling, combined mass and time scaling, or organ-specific allometric scaling, or any of the methods disclosed in Cicone et al., EJNMMI Research (2022) 12, 21; and Konijnenberg et al. J Nucl Med (2004) 45, 1260-1269, the entire contents of both of which is hereby incorporated herein by reference. Biodistribution and radiation dose to specific organs must be carefully assessed and scaled, accounting for the difference in organ sizes and radiation sensitives between species. Safety margins should also be considered due to the potential for radiation damage.

### Outcome

The person skilled in the art will appreciate that there are many measurements by which “treatment” of cancer may be assessed. Merely by way of example, any reduction or prevention of cancer development, cancer progression, cancer recurrence, or cancer propagation may be considered to indicate effective treatment of cancer.

Non-limiting examples of measurable treatment outcomes include reduction in the proportion of tumourigenic cells (e.g. cancer stem cells) within the cancer, and/or inhibition of tumour growth; and/or a reduction in tumour volume. Accordingly, in some embodiments, the at least part of the course of treatment provides one or more of the following outcomes: a reduction in the proportion of tumourigenic cells within the cancer; an inhibition of tumour growth; a reduction in tumour volume; and/or an alleviation and/or control of symptoms associated with the cancer.

In some embodiments, the at least part of the course of treatment provides a reduction in the proportion of tumourigenic cells within the cancer. In some embodiments, the at least part of the course of treatment provides a reduction (in vol %) in the proportion of tumourigenic cells within the cancer of at least about 1, 5, 10, 15, 20, 25, 30, 35, 40, 45, 50, 55, 60, 65, 70, 75, 80, 85, 90, 95, or 99. In some embodiments, the at least part of the course of treatment provides a reduction (in vol %) in the proportion of tumourigenic cells within the cancer of at most about 100, 99, 95, 90, 85, 80, 75, 70, 65, 60, 55, 50, 45, 40, 35, 30, 25, 20, 15, 10, 5, or 1. In some embodiments, the at least part of the course of treatment provides a reduction (in vol %) in the proportion of tumourigenic cells within the cancer in a range provided by any two of the previously described upper and/or lower amounts, for example, the at least part of the course of treatment provides a reduction (in vol %) in the proportion of tumourigenic cells within the cancer of between about 1 to 100, about 20 to 70, about 10 to 30.

In some embodiments, the at least part of the course of treatment provides inhibition of tumour growth. Inhibition of tumour growth is understood to mean that the at least part of the course of treatment at least slows/stunts the tumour growth rate, and in some brings about a reduction in tumour size, resulting in a net beneficial outcome to the patient. The ability of the methods of the present disclosure to bring about a reduction in tumour size, and also to maintain the reduction in tumour size during/after the period in which the treatment is administered represents a particularly relevant indication of effective cancer treatment. As set out in the Examples, the methods of the present disclosure have proven surprisingly effective in this respect. Tumour growth may be assessed by any suitable method in which the change in size of a tumour is assessed over time.

In some embodiments, the at least part of the course of treatment provides an inhibition (in %) of tumour growth of at least about 1, 5, 10, 15, 20, 25, 30, 35, 40, 45, 50, 55, 60, 65, 70, 75, 80, 85, 90, 95, or 99. In some embodiments, the at least part of the course of treatment provides an inhibition (in %) of tumour growth of at most about 100, 99, 95, 90, 85, 80, 75, 70, 65, 60, 55, 50, 45, 40, 35, 30, 25, 20, 15, 10, 5, or 1. In some embodiments, the at least part of the course of treatment provides an inhibition (in %) of tumour growth in a range provided by any two of the previously described upper and/or lower amounts, for example, the at least part of the course of treatment provides an inhibition (in %) of tumour growth of between about 1 to 100, about 20 to 70, about 10 to 30.

Suitably the size of a tumour prior to cancer treatment may be compared with the size of the same tumour during or after cancer treatment. A number of ways in which the size of a tumour may be assessed are known. For example, the size of a tumour may be assessed by imaging (e.g. radiopharmaceutical imaging, such as SPECT/CT imaging) of the tumour in situ within a patient. Suitable techniques, such as imaging techniques, may allow the volume of a tumour to be determined, and changes in tumour volume to be assessed. As shown in the results set out in the examples of this specification, the methods of treatment of the present disclosure are able not only to arrest tumour growth, but are actually able to bring about a reduction in tumour volume in subjects with cancers.

A reduction in tumour volume can be calculated with reference to a suitable control. For example in studies carried out in vitro, or in vivo in suitable animal models, the reduction in tumour volume may be determined by direct comparison between the volume of a tumour treated according to any of the methods described herein and the volume of a control tumour (which may be untreated, or may have received treatment other than according to any of the methods described herein). It will be appreciated that such models requiring lack of treatment of a tumour may not be ethically acceptable in the context of clinical trials or therapeutic management of patients, and in this case a reduction in tumour volume may be assessed by comparing the volume of a treated tumour with the volume of the same tumour prior to treatment, or with a predicted volume that would have been attained by the tumour had no treatment been administered.

In some embodiments, the at least part of the course of treatment provides a reduction in average tumour volume. In some embodiments, the at least part of the course of treatment provides a reduction in average tumour volume (in %) of at least about 1, 5, 10, 15, 20, 25, 30, 35, 40, 45, 50, 55, 60, 65, 70, 75, 80, 85, 90, 95, or 99. In some embodiments, the at least part of the course of treatment provides a reduction in average tumour volume (in %) of at most about 100, 99, 95, 90, 85, 80, 75, 70, 65, 60, 55, 50, 45, 40, 35, 30, 25, 20, 15, 10, 5, or 1. In some embodiments, the at least part of the course of treatment provides a reduction in average tumour volume (in %) in a range provided by any two of the previously described upper and/or lower amounts, for example, the at least part of the course of treatment provides a reduction in average tumour volume (in %) of between about 1 to 100, about 20 to 70, about 10 to 30.

In some embodiments, the at least part of the course of treatment provides an alleviation and/or control of symptoms associated with the cancer. The specific symptoms of a cancer will depend on the particular type of cancer. Non-limiting symptoms of cancer include: fatigue, a lump or thickening that can be felt under the skin, weight changes (unintended weight loss or gain), skin changes (e.g., yellowing, darkening, or redness of the skin, or sores), fever, changes in bowel or bladder habits, persistent cough, difficulty swallowing, hoarseness, persistent indigestion or discomfort after eating, persistent, unexplained muscle or joint pain. Symptoms of prostate cancer include burning or pain during urination, inability to urinate, frequent nocturnal urination, weak urine stream, and blood in urine. In some embodiments, the at least part of the course of treatment provides an alleviation and/or control of symptoms associated with the cancer (in %, as experienced by the patient, or as according to any relevant quantitative measure(s) accepted by persons skilled in the art) of at least about 1, 5, 10, 15, 20, 25, 30, 35, 40, 45, 50, 55, 60, 65, 70, 75, 80, 85, 90, 95, or 99.

Beneficial effects of the methods according to the present disclosure, such as a reduction in the proportion of tumourgenic cells, an inhibition of tumour growth, or reduction in tumour volume, observed on treatment of cancer in accordance with the methods of the present disclosure may be maintained for at least one month. In some embodiments, such beneficial effects may be maintained (in months) for at least about 1, 2, 3, 4, 5, 6, 7, 8, 9, 10, 11, 12, 14, 16, 18, 20, 22, 24, 28, 32, 36, 40, 44, 48, 60, 72, 84, 96, 108 or 120.

In another example, the at least part of the course of treatment provides a change in the levels of one or more biomarkers in a subject which are used to screen for the presence or absence of a cancer. For example, where the subject is being treated for prostate cancer, the at least part of the course of treatment may result in a decrease in levels of prostate-specific antigen (PSA) present in the subject which may indicate reduction in tumour volume as described above.

### Radiopharmaceuticals

The radiopharmaceutical may be any radioactive molecular entity comprising a targeting moiety, a therapeutic radioisotope, and optionally a chelator moiety. In some embodiments, the radiopharmaceutical may be any radioactive molecular entity comprising a targeting moiety, a therapeutic radioisotope and a chelator moiety. Typically, the radiopharmaceutical is a conjugate molecule whereby the chelator moiety is linked to the targeting moiety, either directly (e.g. to a residue of an amino acid forming part of the targeting moiety), or via a linkage derived from e.g. a small molecule or peptide. The chelator moiety is capable of forming a complex with the therapeutic radioisotope. Accordingly, the chelator moiety may contain (or “chelate”) a therapeutic radioisotope (e.g. radioactive cation). A therapeutic radioisotope is a radioisotope that is suitable for use in therapy. In some embodiments, there is no chelator moiety. In some embodiments, the therapeutic radioisotope (e.g. ²¹¹At) is covalently bound to the targeting moiety, optionally via a linkage derived from e.g. a small molecule or peptide reagent used to conjugate the radioisotope.

In some embodiments, the radiopharmaceutical comprises a therapeutic radioisotope described herein, a chelator containing the therapeutic radioisotope, and a targeting moiety specific to a target selectively expressed on cancer cells, wherein the targeting moiety is linked to the chelator. Without limiting possible selections for the targeting moiety, chelator moiety and radioisotope, nor the combinations thereof, the targeting moiety, chelator moiety, and radioisotope may be selected and combined according to any aspect, embodiment, or example thereof as described herein.

Procedures for the synthesis of radiopharmaceuticals are generally known in the art. For example, there are a wide range of moieties which can serve as chelating moieties and which can be conjugated to the targeting moieties of the present disclosure. Moreover, procedures for the complexation of a variety radioisotopes to a variety of chelating moieties are generally known in the art.

### Chelator Moiety

For many shorter half-life radioisotopes, the radiopharmaceutical comprises a chelator for complexing to the radioisotope, viz. in some embodiments, the radiopharmaceutical comprises a chelating moiety. If present, the chelator moiety (also referred to as “chelating agent” or “chelator”) may be any moiety capable of chelating a radioisotope, that is to say, capable of forming a complex. The chelator moiety may be used to avoid dissociation of the radioisotope from the radiopharmaceutical e.g. under mildly acidic conditions, such as within the patient's body. Suitable chelator moieties for diverse range of cations are well known in the art, and can be used in the context of the present disclosure.

In the radiopharmaceutical, the radionuclide metal ion usually forms a non-covalent bond with functional groups of the chelator moiety, non-limiting examples of which include amines or carboxylic acids. The chelator moiety typically has at least two such complexing functional groups to be able to form a chelate complex. The term “chelator moiety” encompasses “polydentate” ligands capable of forming two or more separate coordinate bonds with (“coordinating”) a central (metal) ion, in particular the radionuclide metal ion. Specifically, such molecules or molecules sharing one electron pair may also be referred to as “Lewis bases.” The central (metal) ion is usually coordinated by two or more electron pairs to the chelating agent. The terms, “bidentate”, “tridentate”, and “tetradentate” are known in the art and refer to chelator moieties having two, three, and four electron pairs, respectively, which are readily available for simultaneous donation to a metal ion coordinated by the chelating agent. Usually, the electron pairs of a chelating agent form coordinate bonds with a single central (metal) ion; however, in certain examples, a chelating agent may form coordinate bonds with more than one metal ion, with a variety of binding modes being possible. The terms “coordinating” and “coordination” refer to an interaction in which one multi electron pair donor coordinatively bonds (is “coordinated”) to, i.e. shares two or more unshared pairs of electrons with, one central (metal) ion.

The chelator moiety may be any group suitable for forming a chelate with a radioisotope. Suitable chelator moieties for diverse range of cations are well known in the art, and can be used in the context of the present disclosure. The chelator moiety may be selected such that the chelator moiety forms a square bi-pyramidal complex for complexing the radioisotope. In another embodiment, the chelator moiety does not form a planar or a square planar complex. The chelator moiety may be selected based on its ability to coordinate the desired central (metal) ion, which typically is a radioisotope according to any embodiment or example described herein. The chelator moiety, for example DOTA or DO3AM, may be complexed with any radioisotope (in particular the radioisotopes described herein) as a central (metal) ion. It is within the skill and knowledge of the skilled person in the art to select suitable combinations or chelator moiety and radioisotope.

In some embodiments, the chelator moiety is selected from the group comprising at least one: (i) a macrocyclic ring structure with 8 to 20 ring atoms of which 2 or more are selected from oxygen atoms, sulfur atoms and nitrogen atoms; and (ii) an acyclic, open chain chelating structure with 8 to 20 main chain atoms of which 2 or more are heteroatoms selected from oxygen atoms, sulfur atoms and nitrogen atoms.

In some embodiments, the chelator moiety is selected from the group comprising at least one: (i) a macrocyclic ring structure with 8 to 20 ring atoms of which 3 or more are selected from oxygen atoms, sulfur atoms and nitrogen atoms; and (ii) an acyclic, open chain chelating structure with 8 to 20 main chain atoms of which 3 or more are heteroatoms selected from oxygen atoms, sulfur atoms and nitrogen atoms.

In some embodiments, the chelator moiety is selected from the group comprising at least one: (i) a macrocyclic ring structure with 8 to 20 ring atoms of which 4 or more are selected from oxygen atoms, sulfur atoms and nitrogen atoms; and (ii) an acyclic, open chain chelating structure with 8 to 20 main chain atoms of which 4 or more are heteroatoms selected from oxygen atoms, sulfur atoms and nitrogen atoms.

In some embodiments, the chelator moiety (including any such chelator moiety as described herein) comprises a pendant group capable of coordinating to the therapeutic radioisotope. In some embodiments, the chelator moiety (including any such chelator moiety as described herein) comprises a pendant group selected from the group consisting of acetamide, carboxylic acid, amino, hydroxyl, thiol, phenol, sulfonate, carboxamide and ether. In some embodiments, the chelator moiety (including any such chelator moiety as described herein) comprises a pendant group selected from the group consisting of acetamide and carboxylic acid.

In some embodiments, the chelator moiety (including any such chelator moiety as described herein) comprises a pendant acetamide group, viz, the pendant group is acetamide. In some embodiments, the chelator moiety (including any such chelator moiety as described herein) comprises 2, or at least 2, pendant acetamide groups. In some embodiments, the chelator moiety comprises 3, or at least 3, pendant acetamide groups. In some embodiments, the chelator moiety comprises 4, or at least 4, pendant acetamide groups. There is no particular limitation on the number of pendant acetamide groups that the chelator moiety may comprise, e.g., the chelator moiety may comprise 1, 2, 3, 4, 5, 6, 7, 8 or more pendant acetamide groups. It will be understood that “pendant group” (e.g. pendant acetamide group) refers to an group (e.g. —CH₂CONH₂) that is covalently attached, optionally via a divalent linking moiety (e.g. C₁-C₁₀ alkyl, or PEG group), to the chelator moiety. It will be understand that whether the chelator moiety is a macrocyclic ring structure, or an acyclic open chain chelating structure, that the group (e.g. acetamide group), optionally via the divalent linking moiety, may be attached either to the carbon backbone of the structure, or to a heteroatom such as nitrogen. Examples of chelator moieties comprising at least one pendant acetamide group include, but are not limited to, 2-[4,7,10-tris(2-amino-2-oxoethyl)-1,4,7,10-tetrazacyclododec-1-yl]acetamide (DOTAM), 2-[4,7-bis(2-amino-2-oxoethyl)-1,4,7,10-tetrazacyclododec-1-yl]acetamide (DO3AM) and 1,4,7,10-tetraazacyclododecane-7-acetamide-1,4,10-triacetic acid (PSC). Accordingly, in some embodiments, the chelator moiety is selected from the group consisting of 2-[4,7,10-tris(2-amino-2-oxoethyl)-1,4,7,10-tetrazacyclododec-1-yl]acetamide (DOTAM), 2-[4,7-bis(2-amino-2-oxoethyl)-1,4,7,10-tetrazacyclododec-1-yl]acetamide (DO3AM) and 1,4,7,10-tetraazacyclododecane-7-acetamide-1,4,10-triacetic acid (PSC).

Without intending to be limited by theory, it is believed that the methods described herein are of particularly advantageous application for the administration of radiopharmaceuticals comprising acetamide-based chelators, because it has been surprisingly found that such chelators provide greater retention of the therapeutic isotope, which facilitates toleration by the patient of higher and/or more frequent dosages. It will be appreciated that chelators will have differing propensities to retain a complexed therapeutic radioisotope (e.g. in competition with other chemical species), and that therefore, chelators can exhibit ‘leaching’ of the therapeutic radioisotope (e.g. ²¹²Pb) to differing extents. Leaching of the therapeutic radioisotope will typically lower treatment efficacy (as less of the therapeutic radioisotope may be delivered to the target), and increase toxicity, as the uncomplexed therapeutic radioisotope is free to circulate and more readily build up in particular organs/parts of the body, e.g. the kidney or bone marrow. It has also been surprisingly found that such acetamide-based chelators may alter the pharmacokinetics of a compound, specifically, alter the reabsorption of a compound in the kidneys. It will be appreciated that chelators can have differing propensities to alter pharmacokinetic properties of a compound as the effect is believed to be dictated by a delicate interplay of charge balance and sterics between the chelator and the rest of the molecule. Reabsorption of a compound in kidney tissue will typically lower treatment efficacy (as less of the therapeutic radioisotope may be administered to the patient), and increase toxicity, as the radiation dose to the kidneys is increased.

In some embodiments, the chelating moiety is selected from the group consisting of: 1,4,7,10-tetraazacyclododecane-1,4,7,10-tetraacetic acid (DOTA), N,N″-bis[2-hydroxy-5-(carboxyethyl)-benzyl]ethylenediamine-N,N″-diacetic acid (HBED-CC), 1,4,7-triazacyclononane-1,4,7-triacetic acid (NOTA), 2-(4,7-bis(carboxymethyl)-1,4,7-triazonan-1-yl)pentanedioic acid (NODAGA), 2-(4,7,10-tris(carboxymethyl)-1,4,7,10-tetraazacyclododecan-1-yl)-pentanedioic acid (DOTAGA), 1,4,7-triazacyclononane phosphinic acid (TRAP), 1,4,7-triazacydononane-1-[methyl(2-carboxyethyl)-phosphinic acid]-4,7-bis[methyl(2-hydroxymethyl)phosphinic acid] (NOPO), 3,6,9,15-tetraazabicyclo[9,3,1]pentadeca-1(15), 11,13-triene-3,6,9-triacetic acid (PCTA), N′-{5-[acetyl(hydroxy)amino]pentyl}-N-[5-({4-[(5-aminopentyl) (hydroxy)amino]-4-oxobutanoyl}amino)pentyl]-N-hydroxysuccinamide (DFO), ethylenediaminetetraacetic acid (EDTA), nitrilotriacetic acid (NTA), 1,4,7,10-tetraazacyclododecane-1,4,7-triacetic acid (DO3A), diethylen-etriaminepentaacetic acid (DTPA), and 2-[4,7-bis(2-amino-2-oxoethyl)-1,4,7,10-tetrazacyclododec-1-yl]acetamide (DO3AM), 2-[4,7,10-tris(2-amino-2-oxoethyl)-1,4,7,10-tetrazacyclododec-1-yl]acetamide (DOTAM), bis(carboxymethyl)-1,4,8,11-tetraazabicyclo[6.6.2]hexadecane (CBTE2a), cyclohexyl-1,2-diaminetetraacetic acid (CDTA), 4-(1,4,8,11-tetraazacyclotetradec-1-yl)-methylbenzoic acid (CPTA), 4,11-bis(carboxymethyl)-1,4,8,11-tetraazabicyclo[6.6.2]hexadecan (DO2A), N,N′-dipyridoxylethylendiamine-N,N′-diacetate-5,5,-bis(phosphat) (DPDP), ethyleneglykol-O,O-bis(2-aminoethyl)-N,N,N′,N′-tetraacetic acid (EGTA), N,N-bis(hydroxybenzyl)-ethylenediamine-N,N′-diacetic acid (HBED), hydroxyethyldiaminetriacetic acid (HEDTA), 1-(p-nitrobenzyl)-1,4,7,10-tetraazacyclodecan-4,7,10-triacetate (HP-DOA3), 6-hydrazinyl-N-methylpyridine-3-carboxamide (HYNIC), 1,4,7-triazacyclononan-1-succinic acid-4,7-diacetic acid (NODASA), 1,4,7,10-tetraazacyclododecane-7-acetamide-1,4,10-triacetic acid (PSC), 4,11-bis(carboxymethyl)-1,4,8,11-tetraazabicyclo[6.6.2]hexadecane (TE2A), 1,4,8,11-tetraazacyclododecane-1,4,8,11-tetraacetic acid (TETA), terpyridin-bis(methyleneamintetraacetic acid (TMT), 1,4,7,10-tetraazacyclotridecan-N,N′,N″,N′″-tetraacetic acid (TRITA), triethylenetetraaminehexaacetic acid (TTHA), N,N′-bis[(6-carboxy-2-pyridil)methyl]-4,13-diaza-18-crown-6 (H2macropa) and 4-amino-4-{2-[(3-hydroxy-1,6-dimethyl-4-oxo-1,4-dihydro-pyridin-2-ylmethyl)-carbamoyl]-ethyl}heptanedioic acid bis-[(3-hydroxy-1,6-dimethyl-4-oxo-1,4-dihydro-pyridin-2-ylmethyl)-amide] (THP), and derivatives and/or residues thereof.

In some embodiments, the chelator moiety may be selected from the group consisting of: 1,4,7,10-tetraazacyclododecane-1,4,7,10-tetraacetic acid (DOTA), 2-(4,7-bis(carboxymethyl)-1,4,7-triazonan-1-yl)-pentanedioic acid (NODAGA), 2-[4,7-bis(2-amino-2-oxoethyl)-1,4,7,10-tetrazacyclododec-1-yl]acetamide (DO3AM), 2-[4,7,10-tris(2-amino-2-oxoethyl)-1,4,7,10-tetrazacyclododec-1-yl]acetamide (DOTAM), 1,4,7,10-tetraazacyclododecane-7-acetamide-1,4,10-triacetic acid (PSC), and derivatives and/or residues thereof.

In some embodiments, the chelating agent is DOTA, DO3AM, PSC or DOTAM. In some embodiments, the chelating agent is DOTAM.

Chelator moieties typically have groups on the side chain by which the chelator can be used for attachment to a targeting component of the present invention. Non-limiting examples of such groups include benzylisothiocyanate, maleimide, divinylpyrimidine, or pyridazinedione, by which the chelator moiety can be coupled to, e.g. an amino acid residue of the targeting moiety (e.g. the amino group of a lysine, the sulhydryl group of a cysteine). Various chemical and enzymatic conjugation procedures are known in the art for effecting this conjugation or linking between chelator and targeting moiety.

### Radioisotope

The radiopharmaceuticals described herein comprise a therapeutic radioisotope, particularly those having a half-life of less than about 24 hours. Various radionuclides (radioisotopes) are known to be useful in the field of radiopharmacy. As understood in the art, the term radioisotope refers to isotopes of natural or artificial origin with an unstable neutron to proton ratio that disintegrates with the emission of e.g. an alpha particle (alpha-radiation) or electrons (beta-radiation) or electromagnetic radiation (gamma-radiation). In other words, radioisotopes undergo radioactive decay. The choice of suitable radioisotopes typically depends inter alia on the chemical structure and chelating capability of the chelator moiety (if present), and the intended application of the resulting (complexed) conjugate (e.g. diagnostic vs. therapeutic). For example, the use of DOTA, DOTAM or DO3AM, as the chelator moiety may advantageously enable the use of ²¹³Bi, ²¹²Bi, or ²¹²Pb as radioisotopes. Some radioisotopes, e.g. ²¹¹At, may be covalently conjugated to the targeting moiety, optionally through a linkage derived from e.g. a small molecule or peptide reagent.

Treatment of cancer cells may involve use of emitters (such as e.g., α (alpha), β (beta), γ (gamma) and/or Auger electron emitting radioisotopes) as the radioisotope(s). For example, α-emitting radioisotopes may be delivered to targeted cancer cells by targeting moieties such as PSMA-targeting moieties (i.e. a moiety that binds to or associates with PSMA), which are known in the art. These α-emitting radioisotopes may be of particular interest because they have a high linear energy transfer (LET; which refers to the amount of energy that an ionizing particle transfers to the material traversed per unit distance) compared to other radioisotopes such as ¹⁷⁷Lu, ⁹⁰Y, and/or other β-emitters, and may deposit their high energy within about a 1 to about a 100 μm long pathway tracking within about 1 to about 2 cancer cell clusters. The damaging effects of this high LET radiation may not depend on active cell proliferation or oxygenation, and/or the resulting deoxyribonucleic acid (DNA) damage caused by α-particles may be more difficult to repair than that caused by β-emitting radioisotopes, due to α-particles higher LET. The α-emitting radioisotopes may have an LET that is powerful, and is also generally limited to within the internal region of the cancer cell. The emissions from the α-emitting radioisotopes may also have the ability to cause irreversible damage to the cancer cell that does not require waiting for the life cycle of the cancer cell. Further still, α-emitting radioisotopes can cause death and apoptosis of the cancer cells that developed resistance to β-emitter therapy.

The α-emitting radioisotopes may be, for example, produced during decay of lead based radioisotopes, such as ²¹²Pb radioisotopes. The ²¹²Pb is a β-emitting radioisotope with a half-life of about 10.6 hours with a radioactive emission profile having decay products which are α-emitters having the properties of α-emitting radioisotopes. Since ²¹²Pb decays to ²¹²Bi (which is an α-emitting radioisotope having a half-life of about 60 minutes), which decays whether by α-emission to ²⁰⁸Tl (with a half-life of about 3 min), which decays by β-emission to ²⁰⁸Pb (which is stable), or by β-emission to ²¹²Po (with a half-life of about 0.3 μs), which decays by α-emission to ²⁰⁸Pb.

The use of radioisotopes with short half-lives allows for a higher frequency of dose administration than would be possible (due to e.g. patient safety) for isotopes with longer half-lives. Such a course of treatment, with higher frequency dosing, in theory provides an increased number of emissions that impact the cancerous cells. This leads to a more efficacious treatment without increasing toxicity. In contrast, longer-lived radioisotopes, having half-lives such as those greater than about 24 hours e.g. ¹⁷⁷Lu, can be problematic. For example, radioisotopes with longer half-lives such as ¹⁷⁷Lu remain radioactive for an extended period during which the patient is exposed to radiation.

In some embodiments, the radiopharmaceutical comprises a therapeutic radioisotope for emitting one or more of alpha, beta, gamma, x-ray, and auger electron radiation in an amount effective to damage a cancer cell. In some embodiments, the radiopharmaceutical comprises a therapeutic radioisotope for emitting alpha radiation. In some embodiments, the radiopharmaceutical comprises a therapeutic radioisotope that emits one or more of alpha, beta, gamma, x-ray, and auger electron radiation in an amount effective to damage a cancer cell. In some embodiments, the radiopharmaceutical comprises a therapeutic radioisotope that emits alpha radiation. In some embodiments, the radiopharmaceutical comprises a therapeutic radioisotope that undergoes a series of radioactive decays towards a stable isotope, wherein one or more of the radioactive decays emits alpha radiation.

It will be appreciated that a therapeutic radioisotope may be described according to its radioactive half-life, which is the interval of time required for one half of the atomic nuclei of a given sample of the therapeutic radioisotope to decay. In some embodiments the therapeutic radioisotope has a half-life less than about 48, 24, 20, 16, 12, 8, 4, or 1 hours. In some embodiments the therapeutic radioisotope has a half-life less than about 24 hours. In some embodiments, the therapeutic radioisotope has a half-life (in hours) of between about 1 and about 24, or about 2 and about 24, or about 6 and about 18.

In some embodiments, the therapeutic radioisotope is selected from the group consisting of ⁴³Sc, ⁴⁴Sc, ⁵²Fe, ⁵⁵Co, ⁵⁷Ni, ⁶²Cu, ⁶⁴Cu, ⁶⁶Ga, ⁶⁸Ga, ⁸⁶Y, ⁹⁴Tc, ^{94m}Tc, ^{99m}Tc, ¹⁰⁵Rh, ¹⁰⁹Pd, ¹⁰⁹In, ¹¹⁰In, ^{110m}In, ^{113m}In, ¹²¹Sn, ¹²⁷Te, ¹⁴²Pr, ¹⁴⁹Tb, ¹⁵¹Pm, ¹⁵¹Tb, ¹⁵³Sm, ¹⁶⁵Dy, ¹⁶⁶Ho, ¹⁸⁸Re, ²¹¹At, ²¹²Pb, ²¹²Bi, ²¹³Bi.

In some embodiments, the therapeutic radioisotope is selected from the group consisting of ⁴³Sc, ⁴⁴Sc, ⁵⁵Co, ⁶⁴Cu, ⁸⁶Y, ⁹⁴Tc, ^{99m}Tc, ¹⁵³Sm, ¹⁶⁶Dy, ¹⁸⁸Re, ²¹¹At, ²¹²Pb, ²¹²Bi, and ²¹³Bi.

In some embodiments, the therapeutic radioisotope is selected from the group consisting of ⁶⁴Cu, ¹⁶⁵Dy, ¹⁸⁸Re, ²¹¹At, ²¹²Pb, ²¹²Bi and ²¹³Bi. In some embodiments, the therapeutic radioisotope is selected from the group consisting of ²¹²Pb or ²¹²Bi.

In some embodiments, the therapeutic radioisotope is ²¹²Pb.

### Targeting Moiety

The targeting moiety (which may also referred to as a targeting ligand) is a component that is able to bind to or otherwise associate with a molecular target, for example, a membrane component, a cell surface receptor, a membrane antigen (e.g. PSMA, which is also known as folate hydrolase 1 [FOLH1], glutamate carboxypeptidase II, and NAALADase), or the like. The binding of the targeting molecule to the molecular target may be reversible or irreversible.

The therapeutic, in consequence of the targeting moiety, may become localized or converge at a particular targeted site, for instance, a tumour, a disease site, a tissue, an organ, a type of cell, etc. The targeting moiety may bind, for example, to a disease (e.g., cancer) marker (which is expressed/located at the surface of the cell involved in the disease, e.g. a cancer cell). Thereby, the targeting moiety can guide the radioisotope specifically to the cell involved in the disease, e.g. a cancer cell. As such, the therapeutic may be said to be “target-specific”. Non-limiting examples of targeting moieties include a nucleic acid, peptide, polypeptide, protein (including immunoglobulins and antibodies), glycoprotein, carbohydrate, or lipid. A targeting moiety may be a naturally occurring or synthetic ligand for a cell surface receptor, e.g., a growth factor, hormone, LDL, transferrin, etc. A targeting moiety can be an antibody, which term is intended to include antibody fragments, characteristic portions of antibodies, single chain targeting moieties which can be identified, for example, using procedures such as phage display, cell display or mRNA display. A targeting moiety may also be a targeting peptide, targeting peptidomimetic, or a small molecule, whether naturally-occurring or artificially created (e.g., via chemical synthesis). In some embodiments, a targeting moiety of the radiopharmaceutical is a targeting peptide.

Typically, the molecular target of the targeting moiety is specific for or overexpressed by the target cell (e.g., a cell “marker”). Various molecular targets, to which the targeting molecule may suitably bind, are known in the art. Examples of receptors and cell surface molecules present on tumour cells, which may be a target structure for the targeting molecule, are described herein. However, the target structures are not limited to the receptors and cell surface molecules described below. Further receptors and cell surface molecules present on cancer or other disease cells are contemplated as target structures for the targeting molecules. Moreover, further targeting moieties targeting the receptors and cell surface molecules present on cancer or other disease cells are contemplated.

In some embodiments, the targeting moiety binds to or associates with a target selected from the group consisting of: ABCB5, ABHD2, ADAM22, ADCY8, ADCYAP1R1, ADGRB1, ADORA1, ALOX15B, ALPG, AMER2, AP2S1, AQP4, ASIC1, ATP2B3, ATP8B4, BACE2, BDKRB1, BST2, C11orf24, CACNG4, CACNG7, CADM2, CAIX, CALN1, CANT1, CCDC88A, CCR8, CD200, CD320, CD63, CD84, CD9, CDCP1, CDH1, CDH10, CDH20, CDH22, CDH3, CDH6, CEACAM5, CEACAM6, CEACAM7, CHP1, CHRNA3, CHRNA5, CHRNA6, CHRNA7, CHRNA9, CHRNB4, CLDN15, CLDN16, CLDN2, CLDN3, CLDN4, CLDN6, CNST, CNTN1, CSMD2, CSMD3, CSPG5, CT83, CX3CR1, CXCR4, DIRAS2, DLL3, DNER, DPP10, DSG2, DTNA, EDNRB, ENPP3, EPHA2, EPHB2, ERVMER34-1, ESR1, FAIM2, FAP, FAT1, FGFR3, FLRT1, FMN1, FOLH1 (PSMA), FOLR1, FZD6, GABRA1, GABRA3, GABRG1, GABRR3, GJA8, GJD3, GNGT1, GPC3, GPR151, GPR160, GPR21, GPR32, GPR33, GPR34, GPR35, GPR37, GPR45, GPR62, GPR75, GRIA1, GRIA3, GRIA4, GRID2, GRIK1, GRIK4, GRIN2A, GRIN2D, GRIN3A, GRM1, GRM3, GRM5, GRM8, GSGIL, HCAR1, HTR1F, HTR2C, HTR3A, HTR3D, IGF-1R, IGHD1-1, IGKC, IGLC2, IHH, ITLN1, KCND2, KCNG1, KCNG4, KCNIP1, KCNK9, KIT, KLK14, KLK2, KLK3, LAMP5, LANCL2, LAPTM4B, LINGO1, LOXL2, LPAR3, LPAR4, LRIG1, LRRC55, LRRTM1, LRRTM2, LSAMP, LSR, LY6E, LY6G6D, LYPD1, LZTS1, MAGI2, MAGT1, MAL2, MARCKSL1, MC1R, MDGA2, MEGF10, MEGF11, MELK, MELTF, MMP11, MMP13, MMP16, MMP17, MPL, MRGPRX4, MSLN, MTNR1B, MUC16, MUC21, NCAM1, NECTIN2, NKAIN3, NKAIN4, NLGN1, NLGN3, NOX1, NPSR1, NRAS, NRG3, NRROS, NTRK1, NTRK2, NTSR1, NTSR2, OMG, OPALIN, OPCML, OR10A2, OR10A4, OR10A5, OR10W1, OR13C3, OR13C4, OR13C8, OR13D1, OR13F1, OR13G1, OR13H1, OR14A2, OR14K1, OR1C1, OR1F1, OR1J2, OR1J4, OR1K1, OR1L3, ORIL4, OR1L6, OR1M1, ORIN2, OR2A4, OR2AG2, OR2AK2, OR2B2, OR2D3, OR2G2, OR2G3, OR2I1P, OR2L2, OR2L3, OR2L5, OR4D1, OR4N2, OR51E1, OR51E2, OR52K1, OR52K2, OR5B21, OR5C1, OR6F1, OR6J1, OR7G2, OR9A4, OR9G1, OXTR, PCDH1, PCDH15, PCDHB9, PCDHGC3, PCDHGC4, PIK3R6, PLOD3, PLPPR5, POSTN, PPP3CA, PRAME, PRR7, PRSS21, PSD2, PSENEN, PTPRZ1, QRFPR, RAB33A, RAB44, RACGAP1, RASL10A, RCC2, RET, RFTN2, RHEX, RIT2, RNF43, ROR1, RPE65, RXFP2, S100A6, SDC3, SERINC5, SLC15A2, SLC16A1, SLC39A1, SLC39A11, SLC39A6, SLC50A1, SLC52A2, SLC6A1, SLC6A2, SLCO6A1, SMPDL3B, SSTR2, TAAR1, TBC1D3E, TEC, TESC, TLCD1, TM7SF3, TMEM106A, TMPRSS11D, TNFSF18, TRABD2A, TRAV30, TRBV12-3, TRBV6-8, TRBV7-4, TREM2, TRGC1, TRGV9, TRPM1, TRPM8, TRPV2, TTYH1, TTYH3, UMODL1, UNC5B, UPK3B, VAMP8, VANGL2, VXN, XPR1, and ZPLD1.

In some embodiments, the targeting moiety binds to or associates with a target selected from the group consisting of: AQP4, ATP2B3, ATP8B4, CAIX, CD63, CD84, CD9, CDCP1, CDH3, CDH6, CEACAM5, CEACAM6, CEACAM7, CLDN15, CLDN16, CLDN2, CLDN3, CLDN4, CLDN6, CXCR4, DLL3, ENPP3, EPHA2, EPHB2, ESR1, FAIM2, FAP, FAT1, FGFR3, FOLH1 (PSMA), FOLR1, GPC3, GRIN2A, GRIN2D, GRIN3A, IGF-IR, IGKC, IGLC2, IHH, KIT, KLK14, KLK2, KLK3, LAMP5, LAPTM4B, LOXL2, LSR, LY6E, LY6G6D, LYPD1, LZTS1, MAGI2, MAGT1, MAL2, MARCKSL1, MC1R, MEGF10, MEGF11, MELK, MELTF, MMP11, MMP13, MMP16, MMP17, MSLN, MUC16, MUC21, NOX1, NPSR1, NRAS, NRG3, NRROS, NTRK1, NTRK2, NTSR1, NTSR2, OMG, OXTR, PCDH1, PCDH15, PCDHB9, PCDHGC3, PCDHGC4, PIK3R6, PLOD3, PLPPR5, POSTN, PPP3CA, PRAME, PRR7, PRSS21, PSD2, PSENEN, PTPRZ1, QRFPR, RAB33A, RAB44, RACGAP1, RASL10A, RCC2, RET, RFTN2, RHEX, RIT2, RNF43, ROR1, RPE65, RXFP2, S100A6, SDC3, SERINC5, SLC15A2, SLC16A1, SLC39A1, SLC39A11, SLC39A6, SLC50A1, SLC52A2, SLC6A1, SLC6A2, SLCO6A1, SMPDL3B, SSTR2, TAAR1, TBC1D3E, TEC, TESC, TLCD1, TM7SF3, TMEM106A, TMPRSS11D, TNFSF18, TRABD2A, TRAV30, TRBV12-3, TRBV6-8, TRBV7-4, TREM2, TRGC1, TRGV9, TRPM1, TRPM8, TRPV2, TTYH1, TTYH3, UMODL1, UNC5B, UPK3B, VAMP8, VANGL2, VXN, XPR1, ZPLD1.

In some embodiments, the targeting moiety binds to or associates with a target selected from the group consisting of: AQP4, ATP2B3, CAIX, CDCP1, CDH3, CDH6, CEACAM5, CEACAM6, CEACAM7, CXCR4, DLL3, ENPP3, EPHA2, EPHB2, ESR1, FAIM2, FAP, FAT1, FGFR3, FOLH1 (PSMA), FOLR1, GPC3, IGF-1R, IHH, KLK14, KLK2, KLK3, LAPTM4B, LOXL2, LSR, LY6E, LY6G6D, LYPD1, LZTS1, MARCKSL1, MC1R, MELK, MMP11, MMP13, MMP16, MMP17, MSLN, NTSR1, NOX1, PIK3R6, PLOD3, POSTN, PPP3CA, PRAME, PRSS21, PTPRZ1, RCC2, RNF43, ROR1, S100A6, SDC3, SLC39A6, SSTR2, TREM2, TTYH1, TTYH3, UPK3B, and VAMP8.

In some embodiments, the targeting moiety binds to or associates with a target selected from the group consisting of: CAIX, CDCP1, CEACAM5, CEACAM6, CXCR4, DLL3, ENPP3, EPHA2, EPHB2, FAP, FGFR3, FOLH1 (PSMA), FOLR1, GPC3, IGF-IR, KLK14, KLK2, KLK3, LOXL2, LSR, MC1R, MMP11, MMP13 MSLN, NTSR1, NOX1, PRAME, PTPRZ1, RNF43, ROR1, SSTR2, and TREM2.

In some embodiments, the targeting moiety binds to or associates with a target selected from the group consisting of: ENPP3, FOLH1 (PSMA), FOLR1, KLK2, KLK3, MC1R, MMP11, MMP13, MSLN, NOX1 and SSTR2. In some embodiments, the targeting moiety binds to or associates with a target selected from the group consisting of: FOLH1 (PSMA) and SSTR2.

In some embodiments, the targeting moiety binds to or associates with SSTR2. In some embodiments, the targeting moiety binds to or associates with FOLH1 (PSMA). In other words, the targeting moiety is a PSMA-targeting moiety (i.e. a moiety that binds to or associates with PSMA). In one embodiment, the radiopharmaceutical is a PSMA-targeting radiopharmaceutical. According to some embodiments or examples described herein, separate and more frequent administrations of a [²¹²Pb]Pb-PSMA-targeting radiopharmaceutical to a patient can effectively treat PSMA-positive prostate cancer.

Non-limiting examples of molecules that may be used to administer radioisotope according to the method described herein include ADVC001, DOTAM-PSMA, PSMA-I&T, PSMA-617, CA008, CA009, CA011, CA012, NG001, DOTATATE, DOTATOC, DOTAMTATE, and PSC-PEGn-TOC.

The person skilled in the art will appreciate that there are a variety of molecules (including small molecules, peptides, proteins, etc.) or moieties that are known to be suitable for the targeted delivery of radioisotopes to particular target receptors, by virtue of the molecule or moiety comprising a targeting moiety suitable for targeting the desired target receptor. Some such molecules comprise chelating moieties, and it will be understood by the person skilled in the art, that such molecules may be sourced directly from a commercial supplier, or be may synthesised by a competent synthetic chemist and/or from a contract research organisation. Such molecules may be obtained and/or synthesised in a form complexed with the therapeutic radioisotope, or in their uncomplexed form. A person skilled in the art will be capable of complexing the molecule with the therapeutic radioisotope to form the radiopharmaceutical. It will also be understood that where the molecule or moiety does not comprise a chelating moiety, that it is within the ability of the skilled person to conjugate said molecule or moiety to a chelating moiety, such that the resulting conjugate molecule may be complexed with the therapeutic radioisotope, and administered according to the methods described herein.

Non-limiting examples of molecules that may be used for the targeted delivery of a therapeutic radioisotope towards CAIX according to the method described herein include G250 and cG250.

Non-limiting examples of molecules that may be used for the targeted delivery of a therapeutic radioisotope towards CEACAM5 according to the method described herein include labetuzumab.

Non-limiting examples of molecules that may be used for the targeted delivery of a therapeutic radioisotope towards CEACAM6 according to the method described herein include NY004.

Non-limiting examples of molecules that may be used for the targeted delivery of a therapeutic radioisotope towards CXCR4 according to the method described herein include those described in Yu et al, Molecules (2023) 28 (12), page 4707-2728, the entire contents of which is hereby incorporated herein by reference. Particular examples include, but are not limited to, AMD3100, AMD3465, CB-Bicyclam, RAD1-24, RAD1-52, HZ270-1, RPS-544, RPS-534, RPS-547, MCFB and CPCR4-2, FDG, NOTA-pentixather, NODA-NCS-pentixather, CPCR4.3, DOTA-r-a-ABA-CPCR4, DOTA-r-a-ABA-iodoCPCR4, CXCR4-L, MK007, FRM001, BL01, BL09, NOTA-CP01, BL08, FRM001 and LY2510924.

Non-limiting examples of molecules that may be used for the targeted delivery of a therapeutic radioisotope towards CDCP1 according to the method described herein include DOTA-4A06 and DOTA-10D7.

Non-limiting examples of molecules that may be used for the targeted delivery of a therapeutic radioisotope towards CAIX according to the method described herein include RAYZ-6114 and HYNIC-PEG4-EPH-3.

Non-limiting examples of molecules that may be used for the targeted delivery of a therapeutic radioisotope towards DLL3 according to the method described herein include DTPA-SC16.

Non-limiting examples of molecules that may be used for the targeted delivery of a therapeutic radioisotope towards IGF-1R according to the method described herein include FPX-01a.

Non-limiting examples of molecules that may be used for the targeted delivery of a therapeutic radioisotope towards FAP according to the method described herein include FAPI-46, FAPI-34, FAPI-04, PNT6555 and FAP-2286.

Non-limiting examples of molecules that may be used for the targeted delivery of a therapeutic radioisotope towards FGFR3 according to the method described herein include FPI-1966 and FPI-1967.

Non-limiting examples of molecules that may be used for the targeted delivery of a therapeutic radioisotope towards GPC3 according to the method described herein include RAYZ-8009, GP2633 and NOTA-TJ12P2.

Non-limiting examples of molecules that may be used for the targeted delivery of a therapeutic radioisotope towards KLK3 according to the method described herein include DOTA-hu5A10.

Non-limiting examples of molecules that may be used for the targeted delivery of a therapeutic radioisotope towards MC1R according to the method described herein include MSH and derivatives thereof such as DOTA-Nle-CycMSHₕₑₓ, PSC-C-MCR1 and VMT1. Further examples include those disclosed in WO03006604, AU2005201166A1, U.S. Pat. No. 8,986,651B2, WO2016179529A2, WO2017223565A1, US20220363719A1 and U.S. Pat. No. 11,395,857B2, the entire contents of each of which is hereby incorporated by reference.

Non-limiting examples of molecules that may be used for the targeted delivery of a therapeutic radioisotope towards MSLN according to the method described herein include MSLN-TTCa.

Non-limiting examples of molecules that may be used for the targeted delivery of a therapeutic radioisotope towards NTSR1 according to the method described herein include FBEM-NT, DEG-VS-NT-DOTA-NT, FGlc-NT4, DOTA-NT-20.3, DOTA-LB119, TRAP (NT4)₃, 3BP-227, 3BP-228, 3BP483, 3BP-227, DOTA-CL-156, FAUC 469. Further examples include those described in Maschaer and Prante, Journal of Labelled Compounds and Radiopharmaceuticals (2018) 61 (30 pages 309-325, the entire contents of which is hereby incorporated herein by reference.

Non-limiting examples of molecules that may be used for the targeted delivery of a therapeutic radioisotope towards SSTR2 according to the method described herein include DOTATATE, DOTATOC, DOTAMTATE, PSC-PEGn-TOC, SARTATE, tyr3-octreotide, and DOTA-JR11.

Non-limiting examples of molecules that may be used for the targeted delivery of a therapeutic radioisotope towards PSMA according to the method described herein include ADVC001, DOTAM-PSMA, PSMA-I&T, PSMA-617, CA008, CA009, CA011, CA012, NG001, PSMA-R2, PSMA-TTCa, DOTA-N3-CTT1403, scFvD2B, J591, 07-1A4, BAY2315497, DOTA-DCIBzL, DOTAM-DCIBzL. Further examples include those described in Alati et al. ‘Preclinical Development in ‘Radiopharmaceutical Therapy for Prostate Cancer’, Seminars in Nuclear Medicine (2023) 53 (5), pages 663-686, the entire contents of which is hereby incorporated herein by reference herein.

In another embodiment, the radiopharmaceutical used to administer radioisotope according to the method described herein is a PSMA-targeting radiopharmaceutical. That is, the radiopharmaceutical comprises a conjugate molecule comprising a targeting moiety/ligand that binds to or associates with PSMA. In one embodiment, the PSMA-targeting radiopharmaceutical is a complex of ²¹²Pb and a PSMA-targeting conjugate. In one embodiment, the PSMA-targeting radiopharmaceutical is a complex of ²¹²Pb and a compound related to PSMA-617 structure, such as:

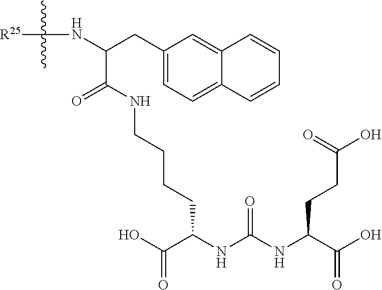

wherein R²⁵ is an optionally linked chelator according to any embodiments or examples thereof as described herein. In one example, the optional linker group may be a C₁₋₂₀alkyl or C₁₋₂₀heteroalkyl, each optionally interrupted and/or optionally substituted with 1-2 groups selected from an aryl, hetaryl, and cycloalkyl. In another example, R25 is a macrocyclic chelator comprising an optionally substituted 1,4,7,10-tetraazacyclododecane group. For example, the 1,4,7,10-tetraazacyclododecane group may be substituted with one or more acetamide groups (e.g. 1,4,7,10-tetraazacyclododecane-1,4,7,10-tetraacetamide, DOTAM). In some examples, R25 is selected from any one of:

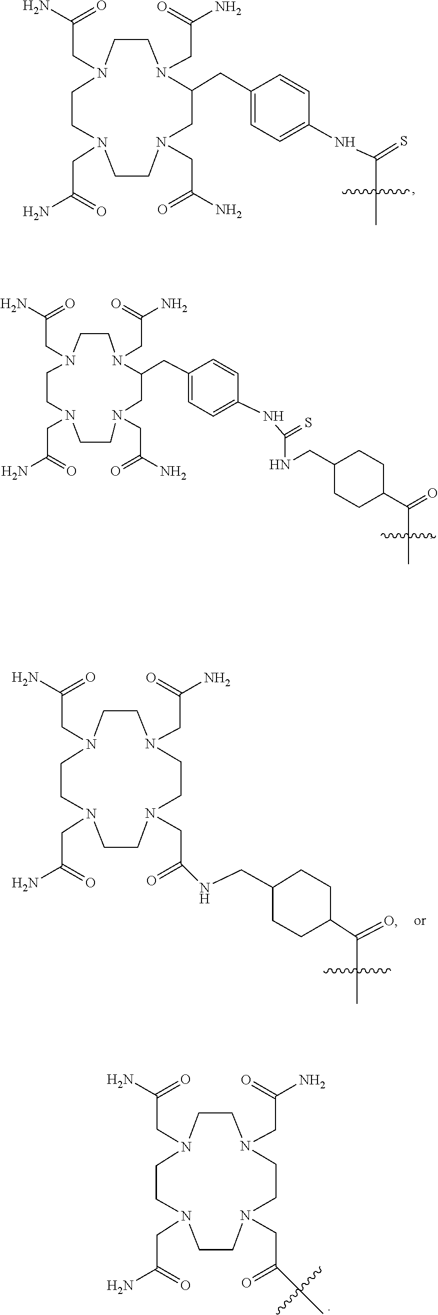 [SMILES: *C(=S)Nc1ccc(CC2CN(CC(N)=O)CCN(CC(N)=O)CCN(CC(N)=O)CCN2CC(N)=O)cc1 ; *C(=O)C1CCC(CNC(=S)Nc2ccc(CC3CN(CC(N)=O)CCN(CC(N)=O)CCN(CC(N)=O)CCN3CC(N)=O)cc2)CC1 ; *C(=O)C1CCC(CNC(=O)CN2CCN(CC(N)=O)CCN(CC(N)=O)CCN(CC(N)=O)CC2)CC1 ; *C(=O)CN1CCN(CC(N)=O)CCN(CC(N)=O)CCN(CC(N)=O)CC1]

For example, the PSMA-targeting radiopharmaceutical can be a complex of ²¹²Pb and a compound having the structure:

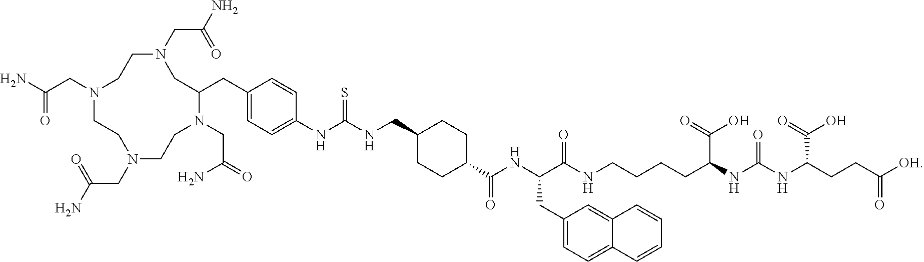 [SMILES: NC(=O)CN1CCN(CC(N)=O)CCN(CC(N)=O)C(Cc2ccc(NC(=S)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)cc2)CN(CC(N)=O)CC1]

In another example, the PSMA-targeting radiopharmaceutical can be a complex of ²¹²Pb and a compound having the structure:

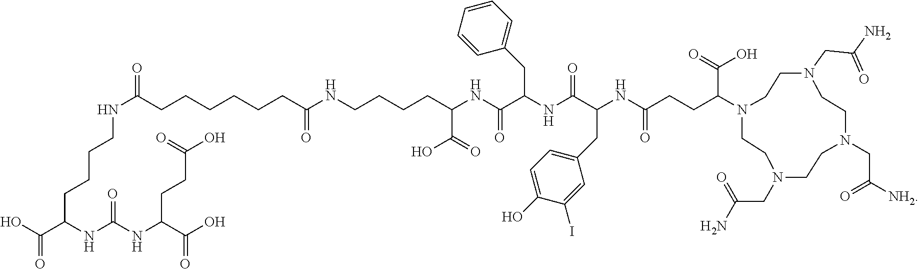 [SMILES: NC(=O)CN1CCN(CC(N)=O)CCN(C(CCC(=O)NC(Cc2ccc(O)c(I)c2)C(=O)NC(Cc2ccccc2)C(=O)NC(CCCCNC(=O)CCCCCCC(=O)NCCCCC(NC(=O)NC(CCC(=O)O)C(=O)O)C(=O)O)C(=O)O)C(=O)O)CCN(CC(N)=O)CC1]

In another embodiment, the PSMA-targeting radiopharmaceutical is a complex of ²¹²Pb and a compound having the structure:

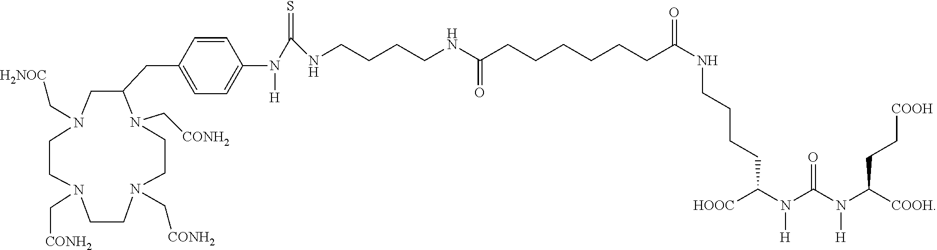 [SMILES: NC(=O)CN1CCN(CC(N)=O)CCN(CC(N)=O)C(Cc2ccc(NC(=S)NCCCCNC(=O)CCCCCCC(=O)NCCCC[C@H](NC(=O)N[C@@H](CCC(=O)O)C(=O)O)C(=O)O)cc2)CN(CC(N)=O)CC1]

In other embodiments, the radiopharmaceutical used to administer radioisotope according to the method described herein is a SSTR2-targeting radiopharmaceutical. That is, the radiopharmaceutical comprises a conjugate molecule comprising a targeting moiety/ligand that binds to or associates with SSTR2. In one embodiment, the SSTR2-targeting radiopharmaceutical is a complex of ²¹²Pb and a SSTR2-targeting conjugate. The SSTR2-targeting conjugate can comprise an optionally linked chelator according to any embodiments or examples thereof as described herein. In one example, the optional linker group may be a C₁₋₂₀alkyl or C₁₋₂₀heteroalkyl, each optionally interrupted and/or optionally substituted with 1-2 groups selected from an aryl, hetaryl, and cycloalkyl. In one example, SSTR2-targeting conjugate comprises a macrocyclic chelator comprising an optionally substituted 1,4,7,10-tetraazacyclododecane group. For example, the 1,4,7,10-tetraazacyclododecane group may be substituted with one or more acetamide groups (e.g. 1,4,7,10-tetraazacyclododecane-1,4,7,10-tetraacetamide, DOTAM). In some examples, the SSTR2-targeting conjugate is selected from, wherein the chelator and optional linker are selected from any embodiments or examples thereof as described herein:

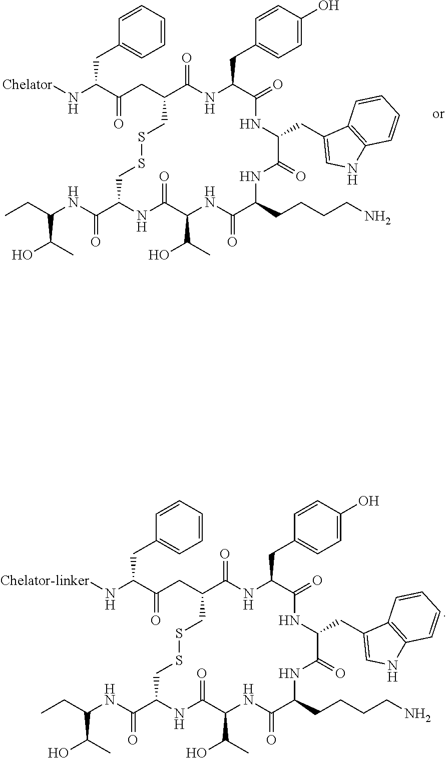

Examples of an SSTR2-targeting radiopharmaceuticals include a complex of ²¹²Pb and a compound having a structure selected from:

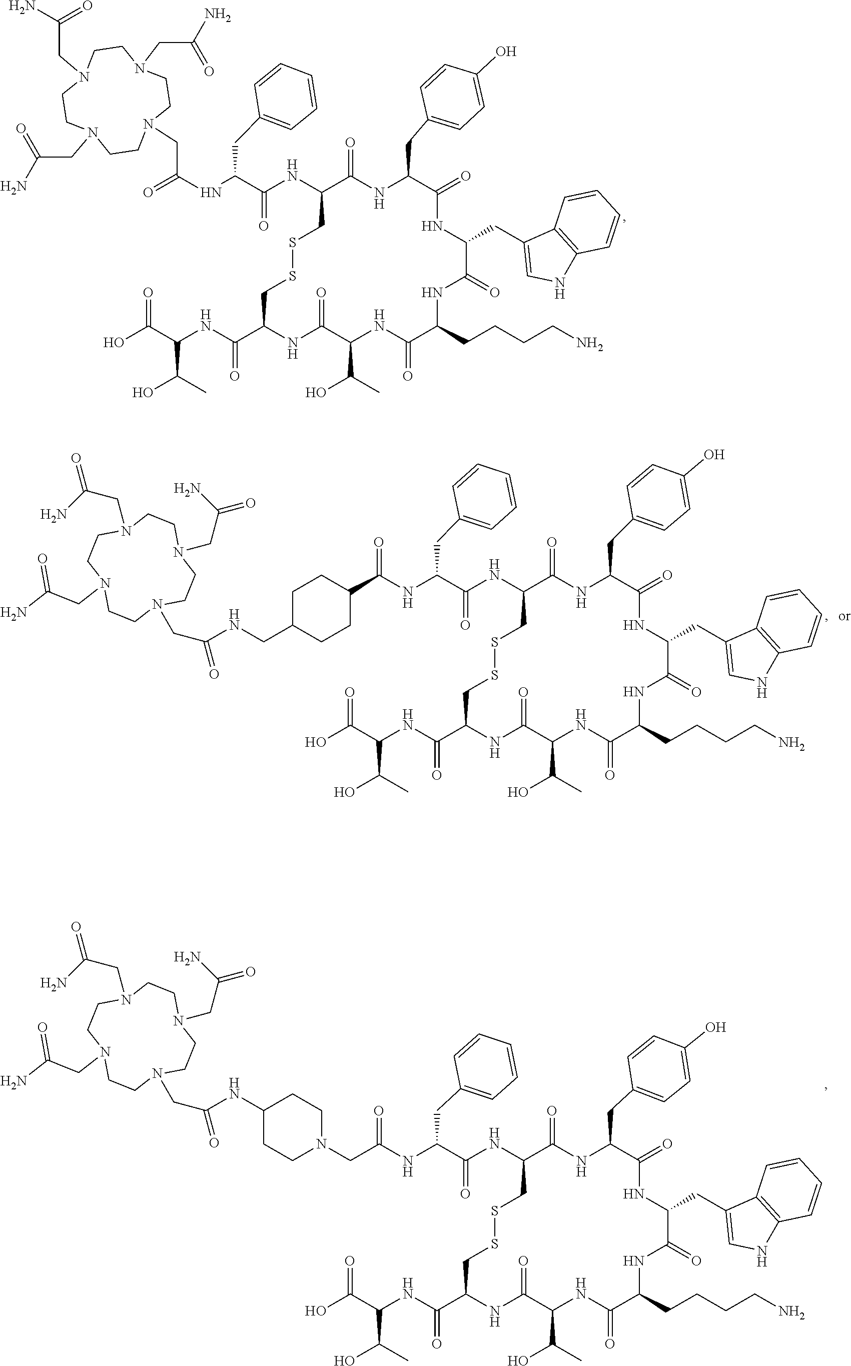

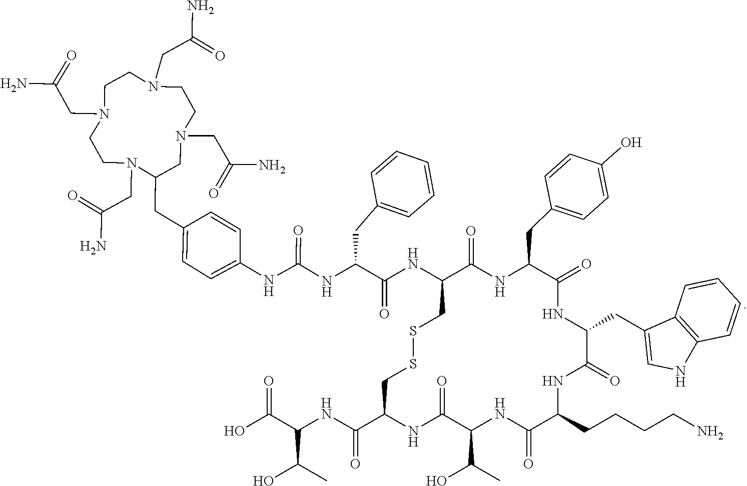 [SMILES: CC(O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](NC(=O)[C@@H](Cc2ccccc2)NC(=O)Nc2ccc(CC3CN(CC(N)=O)CCN(CC(N)=O)CCN(CC(N)=O)CCN3CC(N)=O)cc2)CSSC[C@H](C(=O)NC(C(=O)O)[C@@H](C)O)NC1=O]

Another example of a SSTR2-targeting radiopharmaceutical is a complex of ²¹²Pb and a compound having the structure:

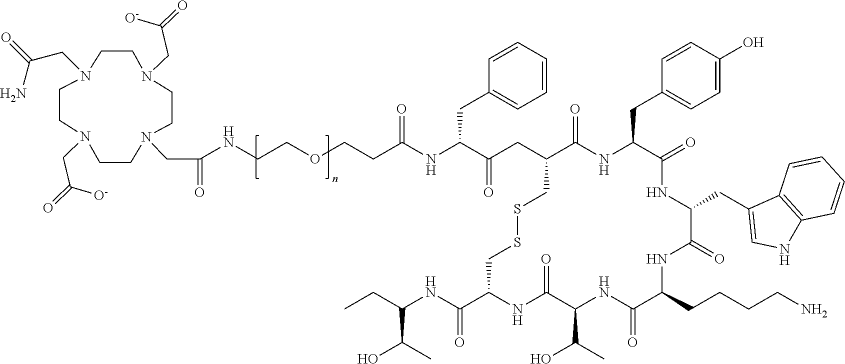

wherein n=0 to 10. In some examples, n=2 to 4.

Still another example of a SSTR2-targeting radiopharmaceutical is a complex of ²¹²Pb and a compound having the structure:

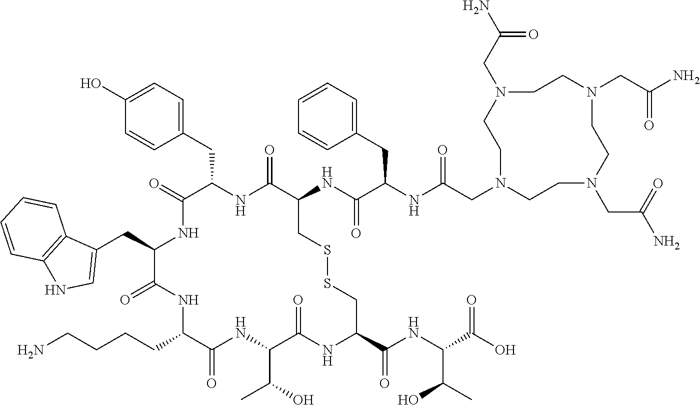 [SMILES: C[C@@H](O)[C@H](NC(=O)[C@@H]1CSSC[C@H](NC(=O)[C@@H](Cc2ccccc2)NC(=O)CN2CCN(CC(N)=O)CCN(CC(N)=O)CCN(CC(N)=O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Suitable conjugate molecules that may be used for the targeted delivery of a therapeutic radioisotope towards PSMA according to the method described herein, include those disclosed in WO2024031155A1, the entire contents of which is hereby incorporated herein by reference.

In some embodiments, the radiopharmaceutical is a complex of a therapeutic radioisotope (e.g. ²¹²Pb) and a compound of Formula (1), or pharmaceutically acceptable salt, solvate or stereoisomer thereof:

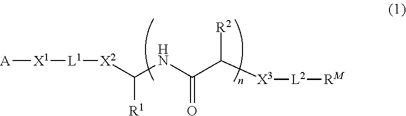

as described in any of the embodiments or examples below.

In the above Formula (1), n may be 0 or an integer from 1 to 10.0 or 1, 2, 3 or more, preferably any integer of 1 to 5 In one embodiment n is 3. When n is 0, it will be understood that there is a directed bond between the central carbon atom attached to R1 and the X³.

In one embodiment, n is 1 and the compound of Formula (1) has a structure of Formula (1A):

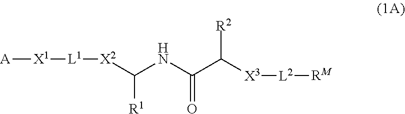

wherein A, L¹, L², X¹, X², X³, R¹, R² and R^{M} are as described herein.

X¹ to X³

In the above Formula (1), X¹ to X³ are each independently absent or selected from the group consisting of —O—, —S—, —C(═O)—, —C(═O)NR³—, —NR³—, —C(═O)O—, —C(═O)S—, —S(═O)₂—, —S(═O)₂NR³— —S(═O)NR³—, —OS(═O)₂—, —N(R³)C(═S)N(R³)— and —N(R³)C(═O)N(R³)—, wherein R³ is described herein. That is, X¹ may be absent, —O—, —S—, —C(═O)—, —C(═O)NR³—, —NR³—, —C(═O)O—, —C(═O)S—, —S(═O)₂—, —S(═O)₂NR³— —S(═O)NR³—, —OS(═O)₂—, —N(R³)C(═S)N(R³)— or —N(R³)C(═O)N(R³)—. Similarly, X² may be absent, —O—, —S—, —C(═O)—, —C(═O)NR³—, —NR³—, —C(═O)O—, —C(═O)S—, —S(═O)₂—, —S(═O)₂NR³— —S(═O)NR³—, —OS(═O)₂—, —N(R³)C(═S)N(R³)— or —N(R³)C(═O)N(R³)—. X³ may be absent, —O—, —S—, —C(═O)—, —C(═O)NR³—, —NR³—, —C(═O)O—, —C(═O)S—, —S(═O)₂—, —S(═O)₂NR³— —S(═O)NR³—, —OS(═O)₂—, —N(R³)C(═S)N(R³)— or —N(R³)C(═O)N(R³)—.

Unless otherwise stated or structurally depicted, it will be appreciated that the orientation of X¹, X² and X³, if present, within the compound of Formula (1) are undefined. That is, X¹, X² and X³, if present, may be attached at either side within the compound of Formula (1). For example, in the above Formula (1), when X² is —C(═O)NH— (i.e. an amide bond), the compound of Formula (1) may have a structure selected from

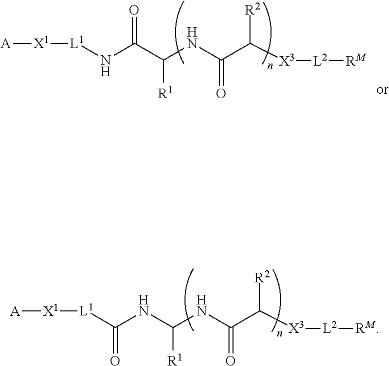

When X¹ is absent, it will be understood that that there is a direct bond between the PSMA-targeting ligand A and the rest of the molecule of Formula (1), including for example a direct bond to any one of the divalent linking moiety L¹, X² and the carbon attached to R¹. Similarly, when X² is absent, it will be understood that there is a direct bond between the carbon atom attached to R¹ and any one of L¹, X¹ and the PSMA-targeting ligand A, depending on whether L¹ and X¹ are present or absent. When X³ is absent, it will be understood that there is a direct bond between the either the central carbon atom attached to R¹ or R² and the divalent linking moiety L² or R^{M}, depending on whether n is 0 or 1 or more, and whether L² is present or absent.

In one embodiment, X¹ to X³ are present and each independently selected from the group consisting —O—, —S—, —C(═O)—, —C(═O)NR³—, —NR³—, —C(═O)O—, —C(═O)S—, —S(═O)₂—, —N(R³)C(═S)N(R³)— and —N(R³)C(═O)N(R³)—. In one embodiment, X¹ to X³ are each —C(═O)NR³—.

In the above Formula (1), where one or more of X¹ to X³ are independently —C(═O)NR³—, —NR³—, —N(R³)C(═S)N(R³)— or —N(R³)C(═O)N(R³)—, each R³ may be independently selected from the group consisting of H, alkyl, alkenyl, alkynyl, carbocyclyl, heterocyclyl, alkylcarbocyclyl, alkylheterocyclyl, each of which is optionally substituted. In one embodiment, where one or more of X¹ to X³ are independently —C(═O)NR³—, —NR³—, —N(R³)C(═S)N(R³)— or —N(R³)C(═O)N(R³)—, each R³ may be independently selected from the group consisting of H and C₁₋₁₀ alkyl. In one embodiment, where one or more of X¹ to X³ are independently —C(═O)NR³—, —NR³—, —N(R³)C(═S)N(R³)— or —N(R³)C(═O)N(R³)—, each R³ is H.

In one embodiment, X¹ to X³ are present and each independently selected from the group consisting of —O—, —S—, —C(═O)—, —C(═O)NH—, —NH—, —C(═O)O—, —C(═O)S—, —S(═O)₂—, —NHC(═S)NH—, and —NHC(═O)NH—. In one embodiment, X¹ to X³ are present and are each —C(═O)NH—.

In one embodiment, X¹ to X³ are each —C(═O)NR³—, and the compound of Formula (1) has a structure of Formula (IB):

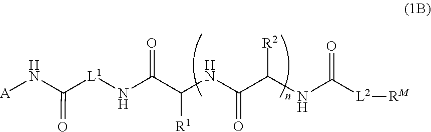

wherein n, A, L¹, L², R¹, R² and R^{M} are as described herein.

L¹ and L²

In the above Formula (1), L¹ and L² represent linker moieties and are each independently absent or a divalent linking moiety. That is, L¹ may be absent or a divalent linking moiety. Unless otherwise stated or structurally depicted, it will be appreciated that the orientation of L¹ and L², if present, within the compound of Formula (1) are undefined. That is, L¹ and L², if present, may be attached at either side within the compound of Formula (1). When L¹ is absent, it will be understood that there is a direct bond between X² and any one of the carbon atom attached to R¹, X¹ and the PSMA targeting ligand A, depending on whether X¹ and X² are present or absent. Similarly, when L² is absent, it will be understood that there is a direct bond between R^{M} and any one of X³, the carbon atom attached to R² (where multiple carbons attached to R² exist [viz., where n is 2 or 3], the bond is to the carbon atom attached to R² that is the furthest from the carbon atom attached to R¹), and the carbon atom attached to R¹, depending on whether X³ is present or absent and n is 0, 1, 2 or 3 etc.).

In one embodiment, L¹ and L² are each independently an aliphatic linker group which is uninterrupted or interrupted and is optionally substituted. As used herein, the term “aliphatic linker group” refers to a divalent linking moiety in which the atoms forming the linking moiety are connected by single, double or triple bonds to form a non-aromatic linking moiety (e.g. does not comprise any aromatic ring structure within the backbone of the linking moiety). In one embodiment, L¹ is present and is —C₁₋₃₀alkyl-, which is uninterrupted or interrupted and optionally substituted. In one embodiment, L¹ is —C₁₋₂₀alkyl- which is uninterrupted or interrupted and optionally substituted.

In one embodiment, L² is absent or is an aliphatic linker group which is uninterrupted or interrupted and optionally substituted. In one embodiment, L² is an aliphatic linker group which is uninterrupted or interrupted and optionally substituted. In one embodiment, L² is absent or is an aliphatic linker group which is uninterrupted or interrupted with one or more groups selected from —O—, —S—, —C(═O)—, —C(═O)NR³—, —NR³—, —C(═O)O—, —C(═O)S—, —S(═O)₂—, —N(R³)C(═S)N(R³)—, and —N(R³)C(═O)N(R³)—. In one embodiment, L² is present and is —C₁₋₂₀alkyl-, which is uninterrupted or interrupted and optionally substituted. In one embodiment, L² is —C₁₋₁₀alkyl- which is uninterrupted or interrupted and optionally substituted.

When present in either L¹ and/or L², each C₁₋₃₀alkyl, C₁₋₂₀alkyl or C₁₋₁₀alkyl may be uninterrupted or interrupted and optionally substituted. In one embodiment, each C₁₋₃₀alkyl, C₁₋₂₀alkyl or C₁₋₁₀alkyl may be uninterrupted or interrupted with one or more groups selected from —O—, —S—, —C(═O)—, —C(═O)NR³—, —NR³—, —C(═O)O—, —C(═O)S—, —S(═O)₂—, —N(R³)C(═S)N(R³)—, and —N(R³)C(═O)N(R³)—, and optionally substituted. In one embodiment, each C₁₋₃₀alkyl, C₁₋₂₀alkyl or C₁₋₁₀alkyl may be uninterrupted or interrupted with one or more groups selected from —O—, —S—, —C(═O)—, —C(═O)NH—, —NH—, —C(═O)O—, —C(═O)S—, —S(═O)₂—, —NHC(═S)NH—, and —NHC(═O)NH—, and optionally substituted. In one embodiment, each C₁₋₃₀alkyl, C₁₋₂₀alkyl or C₁₋₁₀alkyl may be interrupted with a total of 1 to 10, 1 to 5, or preferably 1 to 3 groups, each interruption independently selected from —O—, —S—, —C(═O)—, —C(═O)NR³—, —NR³—, —C(═O)O—, —C(═O)S—, —S(═O)₂—, —N(R³)C(═S)N(R³)—, and —N(R³)C(═O)N(R³)—, and optionally substituted.

In one embodiment, L¹ is —C₁₋₂₀alkyl- which is uninterrupted or interrupted with one or more groups selected from —O—, —S—, —C(═O)—, —C(═O)NH—, —NH—, —C(═O)O—, —C(═O)S—, —S(═O)₂—, —NHC(═S)NH—, and —NHC(═O)NH, and is optionally substituted. In one embodiment, L² is —C₁₋₁₀alkyl- or —C₂₋₁₀alkyl- and is optionally substituted. In one embodiment, L² is —C₂₋₁₀alkyl- and is optionally substituted. In one embodiment, L² is —C₁₋₁₀alkyl- and optionally substituted. In one embodiment, L¹ is —C₁₋₂₀alkyl- which is interrupted with one or more —C(═O)NR³— (e.g. —C(═O)NH—)amide bonds, wherein the C₁₋₂₀alkyl is optionally substituted with one or more R⁸. In one embodiment, L¹ comprises a total of 1 to 5, preferably 1 to 3, more preferably 1 to 2 —C(═O)NR³— (e.g. —C(═O)NH—) amide bond interruptions within its backbone, and optionally substituted.

In one embodiment, L² is optionally substituted with one or more groups selected from C₁₋₁₀alkyl, OC₁₋₁₀alkyl, —NH₂, —OH, —COOH, and —C(═O)OC₁₋₆alkyl. In one embodiment, L² is optionally substituted with one or more groups selected from C₂₋₁₀alkyl, OC₁₋₁₀alkyl, —NH₂, —OH, —COOH, and —C(═O)OC₁₋₆alkyl. In one embodiment, L² is optionally substituted with one or more groups selected from C₁₋₁₀alkyl, C₂₋₁₀alkyl, OC₁₋₁₀alkyl, —NH₂, —OH, —COOH, and —C(═O)OC₁₋₆alkyl.

In line with the above, in a preferred embodiment the moiety —X¹-L¹-X²- in Formula (1) has a structure (L-1):

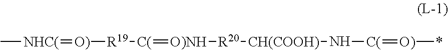

wherein * indicates the bond which is attached to the carbon atom carrying R¹ in Formula (1).

In line with the above, in a preferred embodiment the moiety —X³-L²- in Formula (1) has the structure (L-2) or (L-3):

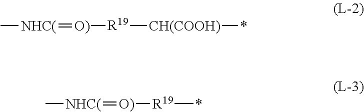

wherein * indicates the bond which is attached to R^{M} in Formula (1).

In some embodiments, R¹⁹ and R²⁰ are each independently selected from an optionally substituted C₁₋₂₀alkyl. In some embodiments, R¹⁹ and R²⁰ are each independently selected from an optionally substituted C₁₋₁₀alkyl. In some embodiments, each of R¹⁹ and R²⁰ may be independently optionally substituted with one or more R⁸. In one embodiment, R¹⁹ and R²⁰ are unsubstituted. In the instance where R¹⁹ or R²⁰ is not substituted with one or more R⁸, then it will be understood that one or more hydrogen atoms will remain at the unsubstituted positions.

In one embodiment, the moiety —X³-L²- in Formula (1L) has the structure (L-2) or (L-3):

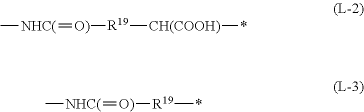

wherein * indicates the bond which is attached to the cyclic Nin Formula (1L).

In some embodiments, R¹⁹ is C₁₋₂₀alkyl. In some embodiments, R¹⁹ is C₁₋₁₀alkyl. In some embodiments, R¹⁹ is C₂₋₂₀alkyl. In some embodiments, R¹⁹ is C₂₋₁₀alkyl. In some embodiments, R¹⁹ is C₁₋₁₀alkyl or C₂₋₁₀alkyl. In some embodiments, R¹⁹ may be optionally substituted with one or more R⁸. In one embodiment, R¹⁹ is unsubstituted. In the instance where R¹⁹ is not substituted with one or more R⁸, then it will be understood that one or more hydrogen atoms will remain at the unsubstituted positions.

In one embodiment, the moiety —X³-L²-in Formula (1L) has the structure (L-2) or (L-3), wherein the carbon atom that is attached to the cyclic N in Formula (1L) is a chiral center. Without intending to limit the scope of any of the aspects, embodiments, or examples described herein, it is believed that the existence of chiral center at the carbon atom that is attached to the cyclic N, may contribute to the PSMA affinity of compounds of Formula (1).

In line with the above, in one embodiment, the moiety —X³-L²-in Formula (1L) has the structure (L-2):

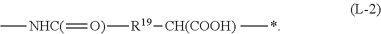

R¹ and R²

Without intending to limit the scope of any of the aspects, embodiments or examples described herein, it is believed that R¹ and/or R² may influence the binding affinity of compounds of Formula (1) to PSMA, through aromatic stacking interaction(s) with an arene-binding site on PSMA. In the above Formula (1), R¹ and each R² are independently selected from the group consisting of an aryl, alkylaryl, heteroaryl and alkylheteroaryl, each of which is optionally substituted. That is, R¹ may be selected from the group consisting of an aryl, alkylaryl, heteroaryl and alkylheteroaryl, each of which is optionally substituted. Similarly, R² may be selected from the group consisting of an aryl, alkylaryl, heteroaryl and alkylheteroaryl, each of which is optionally substituted. R¹ and R² may be the same (e.g. R¹ and R² are both an optionally substituted alkylaryl) or R¹ and R² may be different (e.g. R¹ is an alkylaryl and R² is an alkylaryl substituted with one or more R⁸). In other words, the R¹ and R² substituents are independently selected from one another). In one embodiment, R¹ and R² are each independently an optionally substituted alkylaryl or an optionally substituted alkylheteroaryl. In one embodiment, R¹ and R² are each independently an optionally substituted alkylaryl. In one embodiment, R¹ is an optionally substituted alkylaryl. In one embodiment, R¹ is an optionally substituted alkylheteroaryl. In one embodiment, R² is an optionally substituted alkylaryl. In one embodiment, R² is an optionally substituted alkylheteroaryl. In one embodiment, R¹ and each R² are independently selected from the group consisting of aryl, alkylaryl, heteroaryl and alkylheteroaryl. That is, R¹ may be selected from the group consisting of an aryl, alkylaryl, heteroaryl and alkylheteroaryl. Similarly, R² may be selected from the group consisting of an aryl, alkylaryl, heteroaryl and alkylheteroaryl. In one embodiment, R¹ and R² are each independently selected from an optionally substituted 3-10-membered aryl, an optionally substituted C₁₋₁₀alkyl-3-10-membered aryl, an optionally substituted 3-10-membered heteroaryl, or an optionally substituted C₁₋₁₀alkyl-3-10-membered heteroaryl. That is, R¹ may be selected from an optionally substituted 3-10-membered aryl, an optionally substituted C₁₋₁₀alkyl-3-10-membered aryl, an optionally substituted 3-10-membered heteroaryl, or an optionally substituted C₁₋₁₀alkyl-3-10-membered heteroaryl. Similarly, R² may be selected from an optionally substituted 3-10-membered aryl, an optionally substituted C₁₋₁₀alkyl-3-10-membered aryl, an optionally substituted 3-10-membered heteroaryl, or an optionally substituted C₁₋₁₀alkyl-3-10-membered heteroaryl. In one embodiment, R¹ is an optionally substituted 3-10-membered aryl. In one embodiment, R¹ is an optionally substituted C₁₋₁₀alkyl-3-10-membered aryl. In one embodiment, R¹ is an optionally substituted 3-10-membered heteroaryl. In one embodiment, R¹ is an optionally substituted C₁₋₁₀alkyl-3-10-membered heteroaryl. In one embodiment, R² is an optionally substituted 3-10-membered aryl. In one embodiment, R² is an optionally substituted C₁₋₁₀alkyl-3-10-membered aryl. In one embodiment, R² is an optionally substituted 3-10-membered heteroaryl. In one embodiment, R² is an optionally substituted C₁₋₁₀alkyl-3-10-membered heteroaryl. The heteroaryl or the heteroaryl group of the alkylheteroaryl may selected from the group consisting of pyridyl, pyrimidinyl furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl, each of which is optionally substituted.

In one embodiment, R¹ and R² are each independently an optionally substituted aryl or an optionally substituted alkylaryl. Preferably, the alkylaryl comprises one aryl group bound to an alkyl group, each of which may be optionally substituted. In one embodiment, The aryl or the aryl group of the alkylaryl may be independently selected from phenyl and napthyl, such as 2-naphthyl. The alkyl group of the alkylaryl may be a C₁₋₁₀alkylene group, C₁₋₆alkylene group, or preferable a —CH₂—. In one embodiment, R¹ and R² are each independently an optionally substituted aryl, an optionally substituted benzyl or an optionally substituted-CH₂-naphthyl. In one embodiment, R¹ and R² are each independently an optionally substituted benzyl. In one embodiment, R¹ and R² are benzyl, each independently optionally substituted with one or more groups selected from halogen, —NO₂, —NH₂, —CN, —SCN, —COOH and —OH. In one embodiment, R¹ is benzyl, and R² is benzyl substituted with one or more groups selected from halogen, —NO₂, —NH₂, —CN, —SCN, —COOH and —OH. In one embodiment, R¹ is benzyl and is unsubstituted, and R² is benzyl substituted with one or more groups selected from halogen, —NO₂, —NH₂, —CN, —SCN, —COOH and —OH.

In one embodiment, R¹ and R² are each independently an optionally substituted benzyl, the compound of Formula (1) has a structure of Formula (1C):

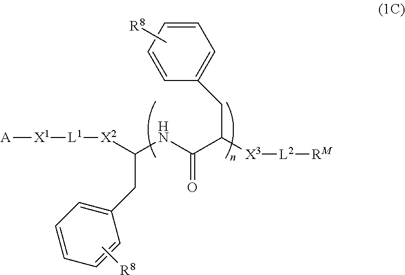

wherein n, A, L¹, L², X¹, X², X³, and R^{M} are as described herein.

In the above Formula (1), each of R¹ and R² may be optionally substituted. In one embodiment, each of R¹ and R² may be optionally substituted with one or more R⁸. In one embodiment, R¹ and R² are each independently optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹, wherein each R¹¹ is independently as described herein. In the instance where R¹ or R² is not substituted with one or more R⁸, then it will be understood that a hydrogen atom will remain as the substitution.

### R³

In the above Formula (1), each R³ may be independently selected from the group consisting of H, alkyl, alkenyl, alkynyl, carbocyclyl, heterocyclyl, alkylcarbocyclyl, and alkylheterocyclyl, each of which is optionally substituted.

In one embodiment, where one or more of X¹ to X³ described herein are independently-C(═O)NR³—, —NR³—, —N(R³)C(═S)N(R³)— or —N(R³)C(═O)N(R³)—, each R³ may be independently selected from the group consisting of H, alkyl, alkenyl, alkynyl, carbocyclyl, heterocyclyl, alkylcarbocyclyl, alkylheterocyclyl, each of which is optionally substituted.

In one embodiment, where R⁴ to R⁷ described herein are each independently selected from the group consisting of —C₁₋₁₀alkylC(═O)N(R³)₂, —C₁₋₁₀alkylP(═O)(OR³)₂, —C₁₋₁₀alkylP(═O)OR³(R³) and —C₁₋₁₀alkylP(═O)(R³)₂, each R³ may be independently selected from the group consisting of H, alkyl, alkenyl, alkynyl, carbocyclyl, heterocyclyl, alkylcarbocyclyl, alkylheterocyclyl, each of which is optionally substituted.

In one embodiment, where L¹ and/or L² described herein comprise an optionally substituted C₁₋₃₀alkyl, optionally substituted C₁₋₂₀alkyl or optionally substituted C₁₋₁₀alkyl, wherein each C₁₋₃₀alkyl, C₁₋₂₀alkyl or C₁₋₁₀alkyl may be uninterrupted or interrupted with one or more groups selected from —O—, —S—, —C(═O)—, —C(═O)NR³—, —NR³—, —C(═O)O—, —C(═O)S—, —S(═O)₂—, —N(R³)C(═S)N(R³)—, and —N(R³)C(═O)N(R³)—, each R³ may be independently selected from the group consisting of H, alkyl, alkenyl, alkynyl, carbocyclyl, heterocyclyl, alkylcarbocyclyl, alkylheterocyclyl, each of which is optionally substituted.

In the above Formula (1), each R³ may be optionally substituted. In one embodiment, each of R³ may be optionally substituted with one or more R⁸. In the instance where R³ is not substituted with one or more R⁸, then it will be understood that a hydrogen atom will remain as the substitution.

### R^{M}

In the above Formula (1), R^{M} is a chelating moiety which is in complex with a therapeutic radioisotope described herein. In some embodiments, the compound of Formula (1) is complexed to a therapeutic radioisotope described herein. For the most part, it will be appreciated that any such complexation occurs predominantly at the chelator moiety of the compound of Formula (1), such as a tetraazamacrocyclic moiety, as described herein. Any disclosure herein that “R^{M} is complexed to a radioisotope” should be understood to also disclose that the compound of Formula (1) is complexed to that radioisotope. R^{M} may be any suitable chelating moiety that is capable of complexing with a radioisotope. In one embodiment, R^{M} is a macrocyclic moiety. In one embodiment, R^{M} is a tetraazamacrocyclic moiety. In one embodiment, R^{M} is chelating moiety having the structure of Formula (M-1):

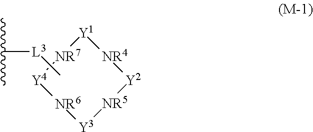

wherein [symbol] 0 represents the bond which attaches R^{M} to the rest of the molecule of Formula (1).

In some embodiments, L³ is absent and the ring is directly connected to the rest of the molecule of Formula (1) via any ring heteroatom (e.g. at any nitrogen atom of M-1) or any one of Y¹ to Y⁴, or L³, or L³ is a divalent linking moiety connecting the ring to the rest of the molecule of Formula (1) via any ring heteroatom or any one of Y¹ to Y⁴. In some embodiments, L³ is absent and the ring is directly connected to the rest of the molecule of Formula (1) via any ring heteroatom (e.g. at any nitrogen atom of M-1) or any one of Y¹ to Y⁴. It will be appreciated that when L³ is absent the rest of the molecule of Formula (1) is connected at any nitrogen atom of M-1, the corresponding R group at that nitrogen atom (e.g. either one of R⁴ to R⁷) will be absent. In some embodiments, L³ is a divalent linking moiety connecting the ring to the rest of the molecule of Formula (1) via any ring heteroatom (e.g. at any nitrogen atom of M-1) or any one of Y¹ to Y⁴. It will be appreciated that when L³ is present and connecting the ring to the rest of the molecule of Formula (1) at any nitrogen atom of M-1, the corresponding R group at that nitrogen atom (e.g. either one of R⁴ to R⁷) will be absent.

It will be appreciated that for compounds of Formula (1), there will always be a bond from the ring (e.g. the macrocyclic chelator) to any one of L³, L², X³, the carbon atom attached to R² (where multiple carbons attached to R² exist [viz., where n is 2 or 3], the bond is to the carbon atom attached to R² that is the furthest from the carbon atom attached to R¹), and the carbon atom attached to R¹. The person skilled in the art will understand that in some embodiments, each of L³, L², X³, and the carbon atom attached to R², may independently be absent or present, and that in consequence, the atom to which the ring is attached may be an atom of or defined by L³, L², X³, or the carbon atom attached to R², or the carbon atom attached to R¹, as the case may be.

More generally, it will be understood that some embodiments permit multiple permutations of absent/present groups (e.g. X¹, X², X³, L¹, L² or L³), and that in such cases a given group will be linked (where relevant) via a covalent bond to the nearest present group, according to the order defined by the relevant structural formulae (e.g. the structure of Formula (1), any one of Formulas (1A) to (1L), and any other structure described herein). This will be clear to the person skilled in the art, and for ease of reference where there are multiple possibilities for attachment depending on group/variable selection, the term ‘the rest of the molecule’ has been adopted in accordance with the above explanation.

In one embodiment, R^{M} is a chelating moiety having structure of formula (M-2):

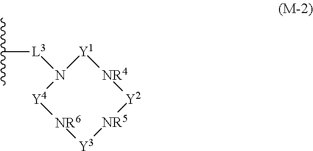

In one embodiment, L³ is absent or —C₁₋₂₀alkyl- which is uninterrupted or interrupted and optionally substituted. In one embodiment, L³ is absent or —C₁₋₁₀alkyl- which is uninterrupted or interrupted and optionally substituted. When present in L³, each C₁₋₂₀alkyl or C₁₋₁₀alkyl may be uninterrupted or interrupted and optionally substituted. In one embodiment, each C₁₋₂₀alkyl or C₁₋₁₀alkyl may be uninterrupted or interrupted with one or more groups selected from —O—, —S—, —C(═O)—, —C(═O)NR³—, —NR³—, —C(═O)O—, —C(═O)S—, —S(═O)₂—, —N(R³)C(═S)N(R³)—, and —N(R³)C(═O)N(R³)—, and optionally substituted. In one embodiment, each C₁₋₂₀alkyl or C₁₋₁₀alkyl may be uninterrupted or interrupted with one or more groups selected from —O—, —S—, —C(═O)—, —C(═O)NH—, —NH—, —C(═O)O—, —C(═O)S—, —S(═O)₂—, —NHC(═S)NH—, and —NHC(═O)NH—, and optionally substituted. In one embodiment, each C₁₋₂₀alkyl or C₁₋₁₀alkyl is uninterrupted and optionally substituted. In one embodiment, L³ may be optionally substituted. In one embodiment, L³ may be optionally substituted with one or more R⁸. In the instance where L³ is not substituted with one or more R⁸, then it will be understood that a hydrogen atom will remain as the substitution.

In some embodiments, Y¹ to Y⁴ are each independently an optionally substituted —C₁₋₆alkyl-. In some embodiments, Y¹ to Y⁴ are each independently selected from an optionally substituted —C₂₋₆alkyl-. In some embodiments, Y¹ to Y⁴ are each independently selected from an optionally substituted —C₂₋₄alkyl-. In some embodiments, Y¹ to Y⁴ are each independently selected from an optionally substituted —C₂₋₃alkyl-. Each alkyl of Y¹ to Y⁴ may be optionally substituted. In one embodiment, each alkyl of Y¹ to Y⁴ may be optionally substituted with one or more R⁸. In the instance where Y¹ to Y⁴ is not substituted with one or more R⁸, then it will be understood that a hydrogen atom will remain as the substitution.

In some embodiments, R⁴ to R⁷ are each independently selected from the group consisting of —C(═O)N(R³)₂, —P(═O)(OR³)₂, —P(═O)OR³(R³), —P(═O)(R³)₂, —C₁₋₁₀alkylC(═O)N(R³)₂, —C¹⁻¹⁰alkylP(═O)(OR³)₂, —C₁₋₁₀alkylP(═O)OR³(R³) and —C₁₋₁₀alkylP(═O)(R³)₂, or one of R⁴ and R⁶ or R⁵ and R⁷ together from a —(CH₂)ₘ— bridge; wherein each C₁₋₁₀alkyl is optionally substituted, or R⁴ to R⁷ is a bond connecting the ring to L³ or the rest of the molecule of Formula (1), and m is 1 to 3, preferably 2.

In some embodiments, R⁴ to R⁷ are each independently selected from the group consisting of —C(═O)N(R¹⁴)₂, —P(═O)(OR¹⁴)₂, —P(═O)OR³(R¹⁴), —P(═O)(R¹⁴)₂, —C₁₋₁₀alkylC(═O)N(R¹⁴)₂, —C₁₋₁₀alkylP(═O)(OR¹⁴)₂, —C₁₋₁₀alkylP(═O)OR¹⁴(R¹⁴) and —C₁₋₁₀alkylP(═O)(R¹⁴)₂, or one of R⁴ and R⁶ or R⁵ and R⁷ together from a —(CH₂)ₘ— bridge; wherein each C₁₋₆alkyl is optionally substituted, or R⁴ to R⁷ is a bond connecting the ring to L³ or the rest of the molecule of Formula (1), and m is 1 to 3, preferably 2.

In some embodiments, R⁴ to R⁷ are each independently selected from the group consisting of —C(═O)NH₂, —P(═O)(OH)₂, —P(═O)(OH) H, —P(═O)(OH)OC₁₋₆alkyl, —C₁₋₆alkylC(═O)NH₂, —C₁₋₆alkylP(═O)(OH)₂, —C₁₋₆alkyl-P(═O)(OH)H, and —C₁₋₆alkyl-P(═O)(OH)OC₁₋₆alkyl, or one of R⁴ and R⁶ or R⁵ and R⁷ together from a —(CH₂)ₘ— bridge; wherein each C₁₋₆alkyl is optionally substituted, or R⁴ to R⁷ is a bond connecting the ring to L³ or the rest of the molecule of Formula (1), and m is 1 to 3, preferably 2.

In some embodiments, R⁴ to R⁷ are each independently selected from the group consisting of —C(═O)N(R¹⁴)₂ and —C₁₋₁₀alkylC(═O)N(R¹⁴)₂, or one of R⁴ and R⁶ or R⁵ and R⁷ together from a —(CH₂)ₘ— bridge, or R⁴ to R⁷ is a bond connecting the ring to L³ or the rest of the molecule of Formula (1); wherein m is 1 to 3, preferably 2.

In some embodiments, R⁴ to R⁷ are each independently selected from the group consisting of —C(═O)NH₂, and —C₁₋₆alkyl-C(═O)NH₂, or one of R⁴ and R⁶ or R⁵ and R⁷ together from a —(CH₂)ₘ— bridge, or R⁴ to R⁷ is a bond connecting the ring to L³ or the rest of the molecule of Formula (1); wherein m is 1 to 3, preferably 2. In some embodiments, each R⁴ to R⁷ may be optionally substituted. In some embodiments, each R⁴ to R⁷ may be optionally substituted with one or more R⁸. In the instance where R⁴ to R⁷ is not substituted with one or more R⁸, then it will be understood that a hydrogen atom will remain as the substitution.

In some embodiments, R^{M} is a chelating moiety having a structure selected from the group consisting of of Formula (M-1A) to (M-1D):

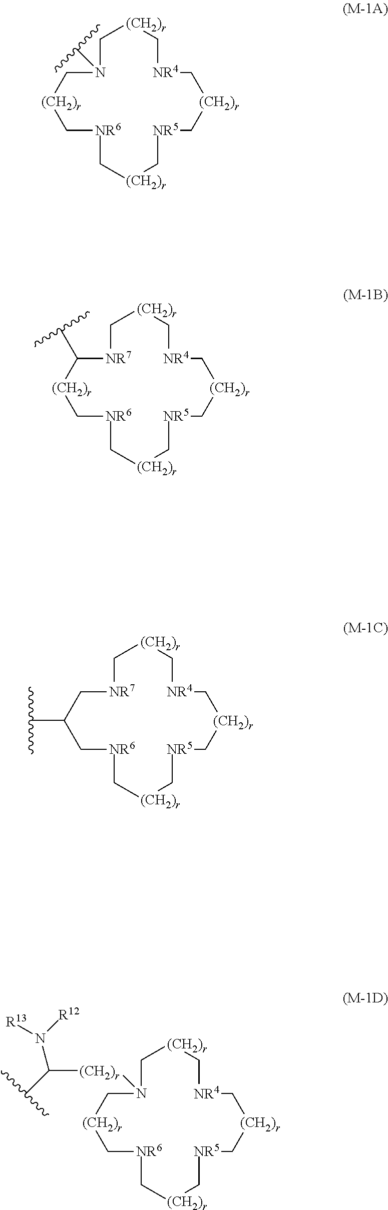

wherein [symbol] represents the bond which attaches R^{M} to the rest of the molecule of in Formula (1); r is 0 or 1, and R⁴ to R⁷ are described herein.

In some embodiments, R¹² and R¹³ are each independently selected from the group consisting of H, —C(═O)OR¹⁴, —C(═O)N(R¹⁴)₂, —C₁₋₆alkylC(═O)OR¹⁴, and —C₁₋₆alkylC(═O)N(R¹⁴)₂, —P(═O)(OR¹⁴)₂—P(═O)OR¹⁴(R¹⁴), —P(═O)(R¹⁴)₂, —C₁₋₆alkylP(═O)OR¹⁴(R¹⁴) and —C₁₋₆alkylP(═O)(R¹⁴)₂, wherein each C₁₋₆alkyl is optionally substituted, or R¹² and R¹³ together form an optionally substituted heterocyclyl. In some embodiments, R¹² and R¹³ are each independently selected from the group consisting of H, —C(═O)OR¹⁴, —C(═O)N(R¹⁴)₂, —C₁₋₆alkylC(═O)OR¹⁴, and —C₁₋₆alkylC(═O)N(R¹⁴)₂, wherein each C₁₋₆alkyl is optionally substituted, or R¹² and R¹³ together form an optionally substituted heterocyclyl. In some embodiments, R¹² and R¹³ are each independently —C(═O)OH or —C(═O)NH₂.

In the above Formula (1), each C₁₋₆alkyl of R¹² or R¹³ may be optionally substituted. In one embodiment, each C₁₋₆alkyl of R¹² or R¹³ may be optionally substituted with one or more R⁸. In the instance where the C₁₋₆alkyl of R¹² or R¹³ is not substituted with one or more R⁸, then it will be understood that a hydrogen atom will remain as the substitution.

In some embodiments, each R¹⁴ is independently selected from the group consisting of H, C₁₋₁₀alkyl, C₂₋₁₀alkenyl, C₂₋₁₀alkynyl, 3-10 membered carbocyclyl, 3-10 membered heterocyclyl, C₁₋₁₀alkyl-3-10-membered carbocyclyl, C₁₋₁₀alkyl-3-10-membered heterocyclyl, wherein each alkyl, alkenyl, alkynyl carbocyclyl, and heterocyclyl is optionally substituted, for example with one or more R⁸. In the instance where R¹⁴ is not substituted with one or more R⁸, it will be understood that a hydrogen atom will remain as the substitution.

In some embodiments, R^{M} is a chelating moiety, having a structure selected from the group consisting of Formula (M-1Ai) to (M-1Dii):

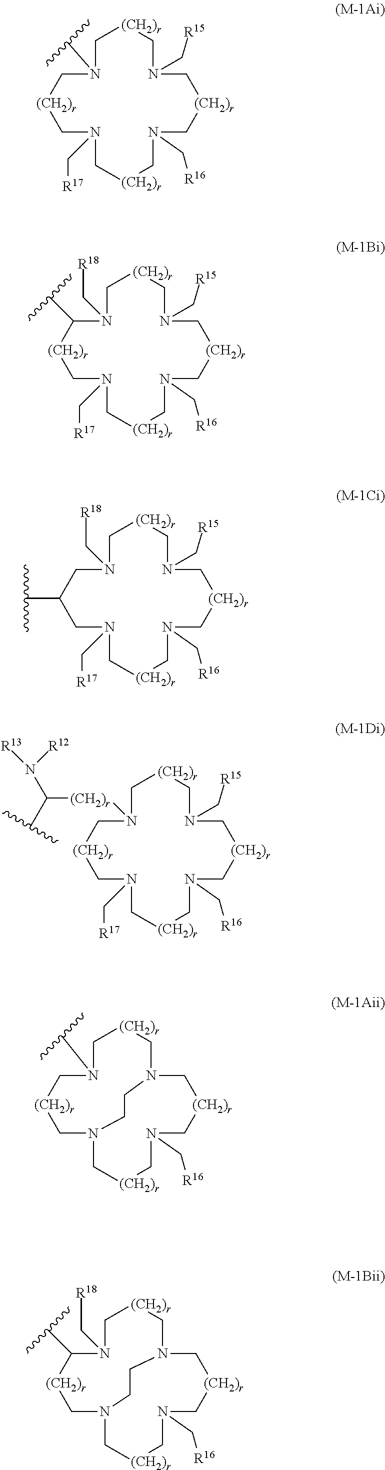

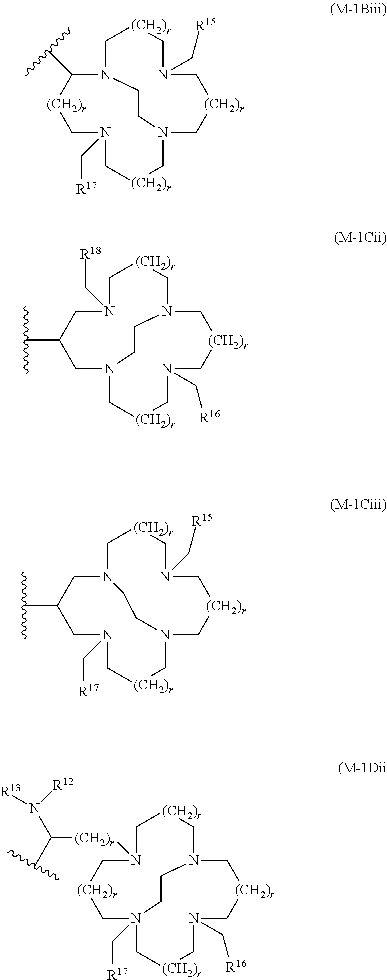

wherein [symbol] represents the bond which attaches R^{M} to the rest of the molecule of in Formula (1); and
r is 0 or 1, and R¹² and R¹³ are described herein.

In some embodiments, R¹⁵ to R¹⁸ are each independently selected from the group consisting of —C(═O)N(R¹⁴)₂, —P(═O)(OR¹⁴)₂, —P(═O)OR¹⁴(R¹⁴), and —P(═O)(R¹⁴)₂. In some embodiments, R¹⁵ to R¹⁸ are each independently selected from the group consisting of —C(═O)NH₂, —P(═O)(OH)₂, —P(═O)(OH) H, —P(═O)(OH)OC₁₋₆alkyl, wherein C₁₋₆alkyl is optionally substituted by one or more R⁸. In the instance where R¹⁵ to R¹⁸ is not substituted with one or more R⁸, it will be understood that a hydrogen atom will remain as the substitution.

In some embodiments, R^{M} is a chelating moiety having a structure of Formula (M-1E), wherein [symbol] represents the bond which attaches R^{M} to the rest of the molecule of in Formula (1):

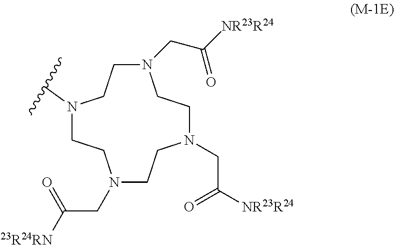

wherein R²³ and R²⁴ are each independently selected from the group consisting of H alkyl, alkenyl, alkynyl, carbocyclyl, heterocyclyl, alkylcarbocyclyl, and alkylheterocyclyl, each of which is optionally substituted; or at least one R²³ and R²⁴ together form an optionally substituted heterocyclyl.

In one embodiment, R²³ and R²⁴ are each independently selected from the group consisting of H, C₁₋₁₀alkyl, C₂₋₁₀alkenyl, C₂₋₁₀alkynyl, 3-10 membered carbocyclyl, 3-10 membered heterocyclyl, C₁₋₁₀alkyl-3-10-membered carbocyclyl, C₁₋₁₀alkyl-3-10-membered heterocyclyl, each of which is optionally substituted; or at least one R²³ and R²⁴ together form an optionally substituted 3-10-membered heterocyclyl. In some embodiments, each R²³ and R²⁴ are independently optionally substituted with one or more R⁸. In the instance where R²³ and R²⁴ is not substituted with one or more R⁸, it will be understood that a hydrogen atom will remain as the substitution.

In one embodiment, R^{M} is a chelating moiety having a structure of Formula (M-1F), wherein [symbol] represents the bond which attaches R^{M} to the rest of the molecule of in Formula (1):

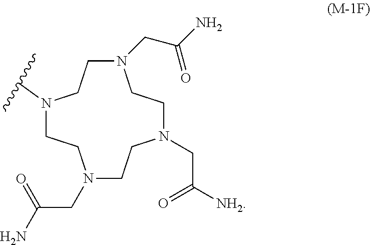 [SMILES: *N1CCN(CC(N)=O)CCN(CC(N)=O)CCN(CC(N)=O)CC1]

In one embodiment, —X³-L²-R^{M} in Formula (1) is 2(R, S)-[1,4,7,10-tetraazacyclododecane-4,7,10-triacetamido]-5-amidopentanoic acid.

R^{M} or the compound of Formula (1) is complexed to a therapeutic radioisotope as described herein. In one embodiment, R^{M} is complexed to ²¹²Pb. In one embodiment, the compound of Formula (1) is complexed to ²¹²Pb.

If R^{M} or the compound of Formula (1) is complexed to a radioisotope described herein, the complex comprising the compound of Formula (1) and radioisotope may be called a radiopharmaceutical. It will be appreciated that reference herein to a compound of Formula (1) being “complexed” to a radioisotope is understood to mean that a radioisotope is complexed to R^{M} or the compound of Formula (1). An example of a compound of Formula (1) complexed to a radioisotope via R^{M} is provided below:

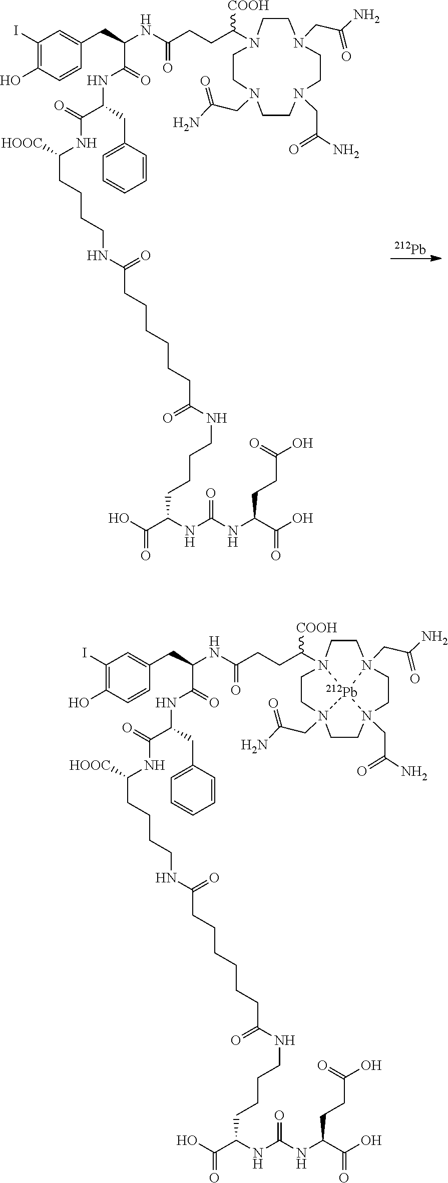

### PSMA Targeting Ligands

In the above Formula (1), A is a PSMA targeting ligand. In one embodiment, A comprises a urea building block (A-1):

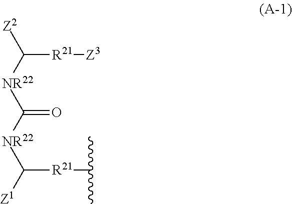

wherein [symbol] represents the bond which attaches A to the rest of molecule in Formula (1).

In some embodiments, R²¹ is —C₁₋₂₀alkyl- which is uninterrupted or interrupted and optionally substituted. In some embodiments, R²¹ is a —C₁₋₁₀alkyl- which is uninterrupted or interrupted and optionally substituted. In some embodiments, R²¹ is a —C₁₋₆alkyl- which is uninterrupted or interrupted and optionally substituted. In some embodiments, R²¹ is optionally substituted with one or more R⁸. In the instance where R²¹ is not substituted with one or more R⁸, it will be understood that a hydrogen atom will remain as the substitution.

In some embodiments, each R²² is independently selected from the group consisting of H, alkyl, alkenyl, alkynyl, carbocyclyl, heterocyclyl, alkylcarbocyclyl, and alkylheterocyclyl, each of which is optionally substituted. In some embodiments, each R²² is H. In some embodiments, each R²² is optionally substituted with one or more R⁸. In the instance where R²² is not substituted with one or more R⁸, it will be understood that a hydrogen atom will remain as the substitution.

In some embodiments, Z¹ to Z³ are each independently selected from the group consisting of —C(═O)OR⁹, —S(═O)OR⁹, —S(═O)₂OR⁹, —S(═O)(OR⁹)₂, —OS(═O)OR⁹, —OS(═O)₂OR⁹, —OS(═O)(OR⁹)₂, —P(═O)(OR⁹)₂, —P(═O)OR⁹ (R⁹), —OP(═O)(OR⁹)₂, and —OP(═O)OR⁹(R⁹). In one embodiment, Z¹ to Z³ are each independently —C(═O)OR⁹. In one embodiment, Z¹ to Z³ are each —COOH.

In some embodiments, A is a PSMA targeting ligand having the structure of Formula (A-2):

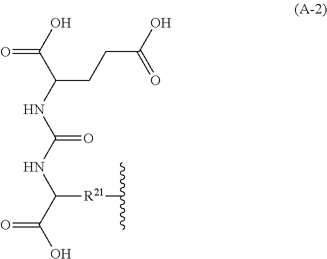

wherein:
[symbol] represents the bond which attaches A to the the rest of the molecule of in Formula (1); and
R²¹ is C₁₋₂₀alkyl optionally substituted with one or more R⁸.
   R⁸ to R¹¹

In the above Formula (1), each of R¹ to R⁷, R¹² to R¹⁴, and R¹⁹ to R²⁴ may be optionally substituted. In one embodiment, each of R¹, R², R³, R⁴, R⁵, R⁶, R⁷, R¹², R¹³, R¹⁴, R¹⁹, R²⁰, R²¹, R²², R²³ and R²⁴ may be optionally substituted with one or more R⁸. In the instance where R¹, R², R³, R⁴, R⁵, R⁶, R⁷, R¹², R¹³, R¹⁴, R¹⁹, R²⁰, R²¹, R²², R²³ and R²⁴ is not substituted with one or more R⁸, then it will be understood that a hydrogen atom will remain as the substitution.

It will be understood that, when any of R¹, R², R³, R⁴, R⁵, R⁶, R⁷, R¹², R¹³, R¹⁴, R¹⁹, R²⁰, R²¹, R²², R²³ and R²⁴ is substituted with one or more R⁸ substituents, the one or more substituents may be the same substituent or a different substituent (e.g., the R⁸ substituents are independently selected from one another).

In the above Formula (1), each of Y¹ to Y⁴ may be optionally substituted. In one embodiment, each of Y¹, Y², Y³, and Y⁴ may be optionally substituted with one or more R⁸. In the instance where Y¹, Y², Y³, and Y⁴ is not substituted with one or more R⁸, then it will be understood that a hydrogen atom will remain as the substitution. In some embodiments, Y¹ is substituted with one, two, three, four, five, or more, R⁸ substituents. In one embodiment, Y¹ is substituted with one R⁸ substituent. In one embodiment, Y¹ is substituted with two R⁸ substituents. In one embodiment, Y¹ is substituted with three R⁸ substituents. In one embodiment, Y¹ is substituted with four R⁸ substituents. In one embodiment, Y¹ is substituted with five R⁸ substituents. In one embodiment, Y¹ is substituted with more than five R⁸ substituents. In one embodiment, Y² is substituted with one R⁸ substituent. In one embodiment, Y² is substituted with two R⁸ substituents. In one embodiment, Y² is substituted with three R⁸ substituents. In one embodiment, Y² is substituted with four R⁸ substituents. In one embodiment, Y² is substituted with five R⁸ substituents. In one embodiment, Y² is substituted with more than five R⁸ substituents. In one embodiment, Y³ is substituted with one R⁸ substituent. In one embodiment, Y³ is substituted with two R⁸ substituents. In one embodiment, Y³ is substituted with three R⁸ substituents. In one embodiment, Y³ is substituted with four R⁸ substituents. In one embodiment, Y³ is substituted with five R⁸ substituents. In one embodiment, Y³ is substituted with more than five R⁸ substituents. In one embodiment, Y⁴ is substituted with one R⁸ substituent. In one embodiment, Y⁴ is substituted with two R⁸ substituents. In one embodiment, Y⁴ is substituted with three R⁸ substituents. In one embodiment, Y⁴ is substituted with four R⁸ substituents. In one embodiment, Y⁴ is substituted with five R⁸ substituents. In one embodiment, Y⁴ is substituted with more than five R⁸ substituents.

In the above Formula (1), each L¹, L² and L³ may be optionally substituted. In one embodiment, each L¹, L² and L³ may be optionally substituted with one or more R⁸. In the instance where L¹, L² and L³ is not substituted with one or more R⁸, then it will be understood that a hydrogen atom will remain as the substitution. In some embodiments, L¹ is substituted with one, two, three, four, five, or more, R⁸ substituents. In one embodiment, L¹ is substituted with one R⁸ substituent. In one embodiment, L¹ is substituted with two R⁸ substituents. In one embodiment, L¹ is substituted with three R⁸ substituents. In one embodiment, L¹ is substituted with four R⁸ substituents. In one embodiment, L¹ is substituted with five R⁸ substituents. In one embodiment, L¹ is substituted with more than five R⁸ substituents. In one embodiment, L² is substituted with one R⁸ substituent. In one embodiment, L² is substituted with two R⁸ substituents. In one embodiment, L² is substituted with three R⁸ substituents. In one embodiment, L² is substituted with four R⁸ substituents. In one embodiment, L² is substituted with five R⁸ substituents. In one embodiment, L² is substituted with more than five R⁸ substituents. In one embodiment, L² is substituted with more than five R⁸ substituents. In one embodiment, L³ is substituted with one R⁸ substituent. In one embodiment, L³ is substituted with two R⁸ substituents. In one embodiment, L³ is substituted with three R⁸ substituents. In one embodiment, L³ is substituted with four R⁸ substituents. In one embodiment, L³ is substituted with five R⁸ substituents. In one embodiment, L³ is substituted with more than five R⁸ substituents.

In the above Formula (1), each R⁸ may be independently selected from the group consisting of H, halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, —NO₂, —CN, —SCN, —N₃, ═O, —N(R⁹)₂, —C(═O)N(R⁹)₂, —S(═O)N(R⁹)₂, —S(═O)₂N(R⁹)₂, —OR⁹, —SR⁹, —OC(═O)R⁹, —C(═O)R⁹, —C(═O)OR⁹, —S(═O)R⁹, —S(═O)₂R⁹, —S(═O)OR⁹, —S(═O)₂OR⁹, —S(═O)(OR⁹)₂, —OS(═O)R⁹, —OS(═O)₂R⁹, —OS(═O)OR⁹,-OS(═O)₂OR⁹, —OS(═O)(OR⁹)₂, —N(R⁹)C(═O)R⁹, —N(R⁹) S(═O)R⁹, —N(R⁹)C(═O)N(R⁹)₂, —N(R⁹) S(═O)₂R⁹, —P(═O)(OR⁹)₂, —P(═O)OR⁹ (R⁹), —P(═O)(R⁹)₂, —OP(═O)(OR⁹)₂, —OP(═O)OR⁹(R⁹) and —OP(═O)(R⁹)₂.

In the above Formula (1), where R⁸ is C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, each C₁₋₁₀alkyl, C₁₋₁₀haloalkyl, C₂₋₁₀alkenyl, C₂₋₁₀alkynyl, 3-10 membered-carbocyclyl, and 3-10-membered-heterocyclyl may be optionally substituted with one or more R¹⁰ substituents. In some embodiments, when R⁸ is C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, each C₁₋₁₀alkyl, C₁₋₁₀haloalkyl, C₂₋₁₀alkenyl, C₂₋₁₀alkynyl, 3-10 membered-carbocyclyl, and 3-10-membered-heterocyclyl may be optionally substituted with one, two, three, four, five or more than five R¹⁰ substituents. It will be understood that, when R⁸ is substituted with one or more R¹⁰ substituents, the one or more substituents may be the same substituent or a different substituent (e.g., the R¹⁰ substituents are independently selected from one another).

In the above Formula (1), each R⁹ may be independently selected from the group consisting of H, C₁-6alkyl, 3-10 membered carbocyclyl, 3-10-membered heterocyclyl, C₁₋₆alkyl-3-10-membered-carbocyclyl, and C₁₋₆alkyl-3-10-membered-heterocyclyl.

In the above Formula (1), when R⁹ is C₁-6alkyl, 3-10 membered carbocyclyl, 3-10-membered heterocyclyl, C₁₋₆alkyl-3-10-membered-carbocyclyl, and C₁₋₆alkyl-3-10-membered-heterocyclyl, each C₁₋₆alkyl, 3-10-membered-carbocyclyl, and 3-10-membered heterocyclyl may be optionally substituted with one or more R¹⁰ substituents. In some embodiments, when R⁹ is C₁-6alkyl, 3-10 membered carbocyclyl, 3-10-membered heterocyclyl, C₁₋₆alkyl-3-10-membered-carbocyclyl, and C₁₋₆alkyl-3-10-membered-heterocyclyl, each C₁₋₆alkyl, 3-10-membered-carbocyclyl, and 3-10-membered heterocyclyl may be optionally substituted with one, two, three, four, five or more than five R¹⁰ substituents. It will be understood that, when R⁹ is substituted with one or more R¹⁰ substituents, the one or more substituents may be the same substituent or a different substituent (e.g., the R¹⁰ substituents are independently selected from one another).

In the above Formula (1), each R¹⁰ may be independently selected from the group consisting of H, halogen, —NO₂, —N(R¹¹)₂, —CN, —SCN, —N₃, ═O, —C(═O)R¹¹, —C(═O)OR¹¹, —C(═O)N(R¹¹)₂, —N(R¹¹)C(═O)R¹¹, —OR¹¹, —P(═O)(OR¹¹)₂, —P(═O)OR¹¹ (R¹¹), —P(═O)(R¹¹)₂, C₁₋₆alkyl, and —OC₁₋₆alkyl. In the above Formula (1), each R¹¹ may be independently selected from the group consisting of H, C₁₋₁₀alkyl, C₁₋₁₀alkyl, C₂₋₁₀alkenyl, C₂₋₁₀alkynyl, 3-10 membered carbocyclyl, 3-10 membered heterocyclyl, C₁₋₁₀alkyl-3-10-membered carbocyclyl, C₁₋₁₀alkyl-3-10-membered heterocyclyl. In one embodiment, each R¹¹ may be independently selected from the group consisting of H and C₁₋₆alkyl. In one embodiment, R¹¹ is H. In one embodiment, R¹¹ is C₁-6alkyl.

In the above Formula (1), each R¹ and R² may be independently optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂,-N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, each R¹ and R² may be independently optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂,-N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R¹ and R² may be independently optionally substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R¹ and R² may be independently optionally substituted with one or more groups selected from halogen, —NO₂, —NH₂, —CN, —SCN, —COOH and —OH.

In one embodiment, R¹ is optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R¹ is optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R¹ is optionally substituted with one or more groups selected from halogen, C₁₋₆alkyl, OC₁₋₆alkyl, C₁-6haloalkyl, OC₁₋₆haloalkyl, —NO₂, —NH₂, —CN, —SCN, —COOH and —OH. In some embodiments, R¹ is optionally substituted with one or more groups selected from halogen, —NO₂, —NH₂, —CN, —SCN, —COOH and —OH. In one embodiment, R¹ is unsubstituted (i.e, substituted with H).

In one embodiment, R¹ is an aryl or alkylaryl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R¹ is an aryl or alkylaryl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R¹ is an aryl or alkylaryl optionally substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R¹ is benzyl optionally substituted with one or more groups selected from halogen, —NO₂, —NH₂, —CN, —SCN, —COOH and —OH. In some embodiments, R¹ is an aryl or alkylaryl optionally substituted with one or more groups selected from halogen, —NO₂, —NH₂, —CN, —SCN, —COOH and —OH. In one embodiment, R¹ is an unsubstituted aryl or an unsubstituted alkylaryl (i.e. ‘substituted’ with H).

In one embodiment, R¹ is phenyl or benzyl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R¹ is phenyl or benzyl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R¹ is phenyl or benzyl optionally substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R¹ is phenyl or benzyl optionally substituted with one or more groups selected from halogen, —NO₂, —NH₂, —CN, —SCN, —COOH and —OH. In some embodiments, R¹ is phenyl or benzyl optionally substituted with one or more groups selected from halogen,-NO₂, —NH₂, —CN, —SCN, —COOH and —OH. In one embodiment, R¹ is an unsubstituted phenyl or unsubstituted benzyl (i.e. ‘substituted’ with H).

In one embodiment, R¹ is benzyl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R¹ is benzyl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R¹ is benzyl optionally substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R¹ is benzyl optionally substituted with one or more groups selected from halogen, —NO₂, —NH₂, —CN, —SCN, —COOH and —OH. In some embodiments, R¹ is benzyl optionally substituted with one or more groups selected from halogen, —NO₂, —NH₂, —CN, —SCN, —COOH and —OH. In one embodiment, R¹ is an unsubstituted benzyl (i.e. ‘substituted’ with H).

In one embodiment, R² is optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R² is optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R² is optionally substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R² is optionally substituted with one or more groups selected from halogen, —NO₂, —NH₂, —CN, —SCN, —COOH and —OH. In one embodiment, R² is optionally substituted with one or more halogen (e.g. one or more of I, Br, C₁, or Br) or —OH.

In one embodiment, R² is an aryl or alkylaryl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R² is an aryl or alkylaryl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R² is an aryl or alkylaryl optionally substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R² is an aryl or alkylaryl optionally substituted with one or more groups selected from halogen, —NO₂, —NH₂, —CN, —SCN, —COOH and —OH. In one embodiment, R² is an aryl or alkylaryl optionally substituted with one or more halogen (e.g. one or more of I, Br, C₁, or Br) or —OH.

In one embodiment, R² is phenyl or benzyl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R² is phenyl or benzyl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R² is phenyl or benzyl optionally substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R² is phenyl or benzyl optionally substituted with one or more groups selected from halogen, —NO₂, —NH₂, —CN, —SCN, —COOH and —OH. In some embodiments, R² is phenyl or benzyl optionally substituted with one or more groups selected from halogen, —NO₂, —NH₂, —CN, —SCN, —COOH and —OH. In one embodiment, R² is phenyl or benzyl optionally substituted with one or more halogen (e.g. one or more of I, Br, C₁, or Br) or —OH.

In one embodiment, R² is benzyl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R² is benzyl optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R² is benzyl optionally substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R² is benzyl optionally substituted with one or more groups selected from halogen, —NO₂, —NH₂, —CN, —SCN, —COOH and —OH. In some embodiments, R² is benzyl optionally substituted with one or more groups selected from halogen, —NO₂, —NH₂, —CN, —SCN, —COOH and —OH. In one embodiment, R² is benzyl optionally substituted with one or more halogen (e.g. one or more of I, Br, C₁, or Br) or —OH.

In one embodiment, R² is an aryl or alkylaryl, substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R² is an aryl or alkylaryl, substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R² is an aryl or alkylaryl, substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R² is an aryl or alkylaryl, substituted with one or more groups selected from halogen, —NO₂, —NH₂, —CN, —SCN, —COOH and —OH. In one embodiment, R² is an aryl or alkylaryl, substituted with one or more halogen (e.g. one or more of I, Br, C₁, or Br) or —OH.

In one embodiment, R² is phenyl or benzyl, substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R² is phenyl or benzyl, substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R² is phenyl or benzyl, substituted with one or more groups selected from halogen, C₁₋₆alkyl, OC₁₋₆alkyl, C₁₋₆haloalkyl, OC₁₋₆haloalkyl, —NO₂, —NH₂, —CN, —SCN, —COOH and —OH. In some embodiments, R² is phenyl or benzyl, substituted with one or more groups selected from halogen, —NO₂, —NH₂, —CN, —SCN, —COOH and —OH. In some embodiments, R² is phenyl or benzyl, substituted with one or more groups selected from halogen, —NO₂, —NH₂, —CN, —SCN, —COOH and —OH. In one embodiment, R² is phenyl or benzyl, substituted with one or more halogen (e.g. one or more of I, Br, C₁, or Br) or —OH.

In one embodiment, R² is benzyl substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰. In some embodiments, R² is benzyl substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R¹¹, —C(═O)OR¹¹, —N(R¹¹)C(═O)R¹¹, and —OR¹¹. In some embodiments, R² is benzyl substituted with one or more groups selected from halogen, C₁₋₆alkyl, OC₁₋₆alkyl, C₁-6haloalkyl, OC₁₋₆haloalkyl, —NO₂, —NH₂, —CN, —SCN, —COOH and —OH. In some embodiments, R² is benzyl substituted with one or more groups selected from halogen, —NO₂, —NH₂, —CN, —SCN, —COOH and —OH. In some embodiments, R² is benzyl substituted with one or more groups selected from halogen, —NO₂, —NH₂, —CN, —SCN, —COOH and —OH. In one embodiment, R² is benzyl substituted with one or more halogen (e.g. one or more of I, Br, C₁, or Br) or —OH.

In one embodiment, R¹ is a group of the formula:

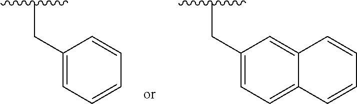 [SMILES: *Cc1ccccc1 ; *Cc1ccc2ccccc2c1]

and R² is a group of the formula:

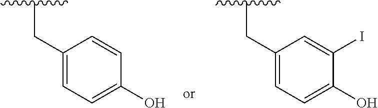

wherein [symbol] represents the bond which attaches R¹ and R², respectively, to the remainder of the compound of Formula (1).

In one embodiment, R¹ is a group of the formula:

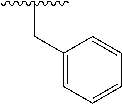 [SMILES: *Cc1ccccc1]

and R² is a group of the formula:

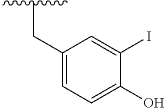

wherein [symbol] represents the bond which attaches R¹ and R², respectively, to the remainder of the compound of Formula (1).

In the above Formula (1), each R³ may be independently optionally substituted with one or more groups H, C₁₋₁₀alkyl, OC₁₋₁₀alkyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, —N(R⁹)₂, —C(═O)N(R⁹)₂, —OR⁹, —OC(═O)R⁹, —C(═O)R⁹, —C(═O)OR⁹, and —N(R⁹)C(═O)R⁹, wherein each C₁₋₁₀alkyl, C₁₋₁₀haloalkyl, C₂₋₁₀alkenyl, C₂₋₁₀alkynyl, 3-10 membered-carbocyclyl, and 3-10-membered-heterocyclyl is optionally substituted with one or more R¹⁰. In some embodiments, each of R³ may be independently optionally substituted with one or more groups selected from C₁₋₁₀alkyl, OC₁₋₁₀alkyl, —NH₂, —OH, —COOH, —C(═O)OC₁₋₆alkyl. In one embodiment, each of R³ is unsubstituted (i.e, substituted with H).

In the above Formula (1), R⁴ to R⁷ may be each independently optionally substituted with one or more groups selected from C₁₋₁₀alkyl, OC₁₋₁₀alkyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, —N(R⁹)₂, —C(═O)N(R⁹)₂, —OR⁹, —OC(═O)R⁹, —C(═O)R⁹, —C(═O)OR⁹, and —N(R⁹)C(═O)R⁹, wherein each C₁₋₁₀alkyl, C₁₋₁₀haloalkyl, C₂₋₁₀alkenyl, C₂₋₁₀alkynyl, 3-10 membered-carbocyclyl, and 3-10-membered-heterocyclyl is optionally substituted with one or more R¹⁰. In one embodiment, R⁴ to R⁷ are unsubstituted (i.e. ‘substituted’ with H).

In the above Formula (1), R¹² and R¹³ may be each independently optionally substituted with one or more groups selected from C₁₋₁₀alkyl, OC₁₋₁₀alkyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, —N(R⁹)₂, —C(═O)N(R⁹)₂, —OR⁹, —OC(═O)R⁹, —C(═O)R⁹, —C(═O)OR⁹, and —N(R⁹)C(═O)R⁹, wherein each C₁₋₁₀alkyl, C₁₋₁₀haloalkyl, C₂₋₁₀alkenyl, C₂₋₁₀alkynyl, 3-10 membered-carbocyclyl, and 3-10-membered-heterocyclyl is optionally substituted with one or more R¹⁰. In one embodiment, R¹² and R¹³ are unsubstituted (i.e. ‘substituted’ with H).

In the above Formula (1), each R¹⁴ may be independently optionally substituted with one or more groups selected from halogen, C₁₋₁₀alkyl, OC₁₋₁₀alkyl, C₁₋₁₀haloalkyl, OC₁₋₁₀haloalkyl, C₂₋₁₀alkenyl, C₂₋₁₀alkynyl, OC₂₋₁₀alkenyl, OC₂₋₁₀alkynyl, —NO₂, —N(R¹¹)₂, —CN, —SCN, —N₃, ═O, —C(═O)R⁹, —C(═O)OR⁹, —N(R⁹)C(═O)R⁹, and —OR⁹, wherein each C₁₋₁₀alkyl, C₁₋₁₀haloalkyl, C₂₋₁₀alkenyl and C₂₋₁₀alkynyl is optionally substituted with one or more R¹⁰.

In the above Formula (1), R¹⁹ to R²⁴ may be each independently optionally substituted with one or more groups selected from C₁₋₁₀alkyl, OC₁₋₁₀alkyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, —N(R⁹)₂, —C(═O)N(R⁹)₂, —OR⁹, —OC(═O)R⁹, —C(═O)R⁹, —C(═O)OR⁹, and —N(R⁹)C(═O)R⁹, wherein each C₁₋₁₀alkyl, C₁₋₁₀haloalkyl, C₂₋₁₀alkenyl, C₂₋₁₀alkynyl, 3-10 membered-carbocyclyl, and 3-10-membered-heterocyclyl is optionally substituted with one or more R¹⁰. In some embodiments, each of R¹⁹ and R²⁴ may be independently optionally substituted with one or more groups selected from C₁₋₁₀alkyl, OC₁₋₁₀alkyl, —NH₂, —OH, —COOH, —C(═O)OC₁₋₆alkyl. In one embodiment, each of R¹⁹ and R²⁴ are unsubstituted (i.e. ‘substituted’ with H).

In the above Formula (1), each of L¹, L² and L³ may be independently optionally substituted with one or more groups selected from R⁸. In some embodiments, each of L¹, L² and L³ may be independently optionally substituted with one or more groups selected from C₁₋₁₀alkyl, OC₁₋₁₀alkyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, —N(R⁹)₂, —C(═O)N(R⁹)₂, —OR⁹, —OC(═O)R⁹, —C(═O)R⁹, —C(═O)OR⁹, and —N(R⁹)C(═O)R⁹, wherein each C₁₋₁₀alkyl, C₁₋₁₀haloalkyl, C₂₋₁₀alkenyl, C₂₋₁₀alkynyl, 3-10 membered-carbocyclyl, and 3-10-membered-heterocyclyl is optionally substituted with one or more R¹⁰. In some embodiments, each of L¹, L² and L³ may be independently optionally substituted with one or more groups selected from C₁₋₁₀alkyl, OC₁₋₁₀alkyl, —N(R⁹)₂, —C(═O)N(R⁹)₂, —OR⁹, —OC(═O)R⁹, —C(═O)R⁹, —C(═O)OR⁹, and —N(R⁹)C(═O)R⁹. In some embodiments, each of L¹, L² and L³ may be independently optionally substituted with one or more groups selected from C₁₋₁₀alkyl, OC₁₋₁₀alkyl, —NH₂, —OH, —COOH, —C(═O)OC₁₋₆alkyl. In one embodiment, each of L¹, L² and L³ may be independently optionally substituted with one or more —NH₂, —OH or —COOH. In one embodiment, each of L¹, L² and L³ may be independently optionally substituted with one or more —COOH.

In the above Formula (1), each of L¹, L² and L³ may each be independently optionally substituted with one or more groups selected from R⁸. In some embodiments, each of L¹, L² and L³ may be independently optionally substituted with one or more groups selected from C₁₋₁₀alkyl, OC₁₋₁₀alkyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, —N(R⁹)₂, —C(═O)N(R⁹)₂, —OR⁹, —OC(═O)R⁹, —C(═O)R⁹, —C(═O)OR⁹, and —N(R⁹)C(═O)R⁹, wherein each C₁₋₁₀alkyl, C₁₋₁₀haloalkyl, C₂₋₁₀alkenyl, C₂₋₁₀alkynyl, 3-10 membered-carbocyclyl, and 3-10-membered-heterocyclyl is optionally substituted with one or more R¹⁰. In some embodiments, L¹ and L² may each be independently optionally substituted with one or more groups selected from C₁₋₁₀alkyl, OC₁₋₁₀alkyl, —N(R⁹)₂, —C(═O)N(R⁹)₂, —OR⁹, —OC(═O)R⁹, —C(═O)R⁹, —C(═O)OR⁹, and —N(R⁹)C(═O)R⁹. In some embodiments, L¹ and L² may each be independently optionally substituted with one or more groups selected from C₁₋₁₀alkyl, OC₁₋₁₀alkyl, —NH₂, —OH, —COOH, —C(═O)OC₁₋₆alkyl. In one embodiment, each of L¹, L² and L³ may be independently optionally substituted with one or more —NH₂, —OH or —COOH. In one embodiment, L¹ and L² may each optionally substituted with one or more —COOH.

In one embodiment, L¹ is —C₁₋₂₀alkyl- which is uninterrupted or interrupted with one or more groups selected from —O—, —S—, —C(═O)—, —C(═O)NH—, —NH—, —C(═O)O—, —C(═O)S—, —S(═O)₂—, —NHC(═S)NH—, and —NHC(═O)NH, and is optionally substituted with one or more groups selected from C₁₋₁₀alkyl, OC₁₋₁₀alkyl, —NH₂, —OH, —COOH, and —C(═O)OC₁₋₆alkyl. In one embodiment, L¹ is —C₁₋₂₀alkyl-interrupted with one or more-C(═O)NR³— (e.g. —C(═O)NH—) amide bonds, and is optionally substituted with with one or more groups selected from C₁₋₁₀alkyl, OC₁₋₁₀alkyl, —NH₂, —OH, —COOH, —C(═O)OC₁₋₆alkyl. In one embodiment, L¹ is —C₁₋₂₀alkyl-interrupted with one or more —C(═O)NR³— (e.g. —C(═O)NH—) amide bonds, and is optionally substituted with with one or more groups selected from —NH₂, —OH, or —COOH. In one embodiment, L¹ is —C₁₋₂₀alkyl-interrupted with one or more —C(═O)NR³— (e.g. —C(═O)NH—) amide bonds, and is optionally substituted with with one or more —COOH.

In one embodiment, L² is —C₁₋₂₀alkyl- which is uninterrupted or interrupted with one or more groups selected from —O—, —S—, —C(═O)—, —C(═O)NH—, —NH—, —C(═O)O—, —C(═O)S—, —S(═O)₂—, —NHC(═S)NH—, and —NHC(═O)NH, and is optionally substituted with one or more groups selected from C₁₋₁₀alkyl, OC₁₋₁₀alkyl, —NH₂, —OH, —COOH, and —C(═O)OC₁₋₆alkyl. In one embodiment, L² is —C₁₋₂₀alkyl-optionally substituted with with one or more groups selected from C₁₋₁₀alkyl, OC₁₋₁₀alkyl, —NH₂, —OH, —COOH, —C(═O)OC₁₋₆alkyl. In one embodiment, L² is —C₁₋₂₀alkyl-optionally substituted with with one or more groups selected from —NH₂, —OH, or —COOH. In one embodiment, L² is —C₁₋₂₀alkyl-optionally substituted with with one or more —COOH.

In one embodiment, L³ is —C₁₋₂₀alkyl- or —C₁₋₁₀alkyl-, which is uninterrupted or interrupted with one or more groups selected from O—, —S—, —C(═O)—, —C(═O)NH—, —NH—, —C(═O)O—, —C(═O)S—, —S(═O)₂—, —NHC(═S)NH—, and —NHC(═O)NH, and optionally substituted with one or more groups selected from C₁₋₁₀alkyl, OC₁₋₁₀alkyl, 3-10 membered carbocyclyl, 3-10-membered heterocyclyl, C₁₋₁₀alkyl-3-10-membered-carbocyclyl, C₁₋₁₀alkyl-3-10-membered-heterocyclyl, —N(R⁹)₂, —C(═O)N(R⁹)₂, —OR⁹, —OC(═O)R⁹, —C(═O)R⁹, —C(═O)OR⁹, and —N(R⁹)C(═O)R⁹, wherein each C₁₋₁₀alkyl, C₁₋₁₀haloalkyl, C₂₋₁₀alkenyl, C₂₋₁₀alkynyl, 3-10 membered-carbocyclyl, and 3-10-membered-heterocyclyl is optionally substituted with one or more R¹⁰.

### Compounds of Formula (1)

In some embodiments, the compound of Formula (1) has a structure selected from the following Formula (1C) to (1H)

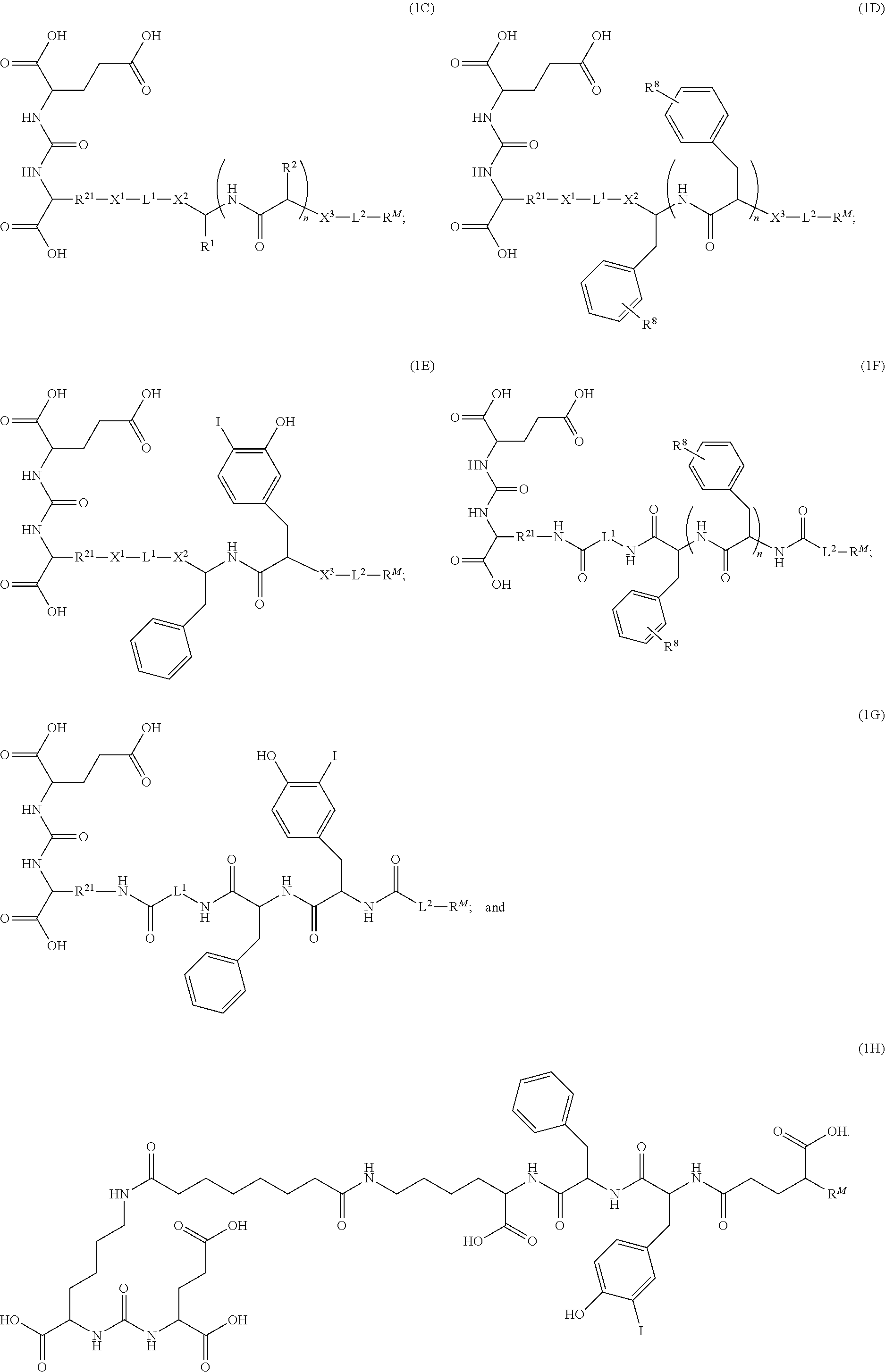

or a pharmaceutically acceptable salt, solvate or stereoisomer thereof;
wherein n, L¹, L², X¹, X², X³, R¹, R², R²¹ and R^{M} are as described herein.

In some embodiments, the compound of Formula (1) has a structure selected from the following Formula (II) to (1K):

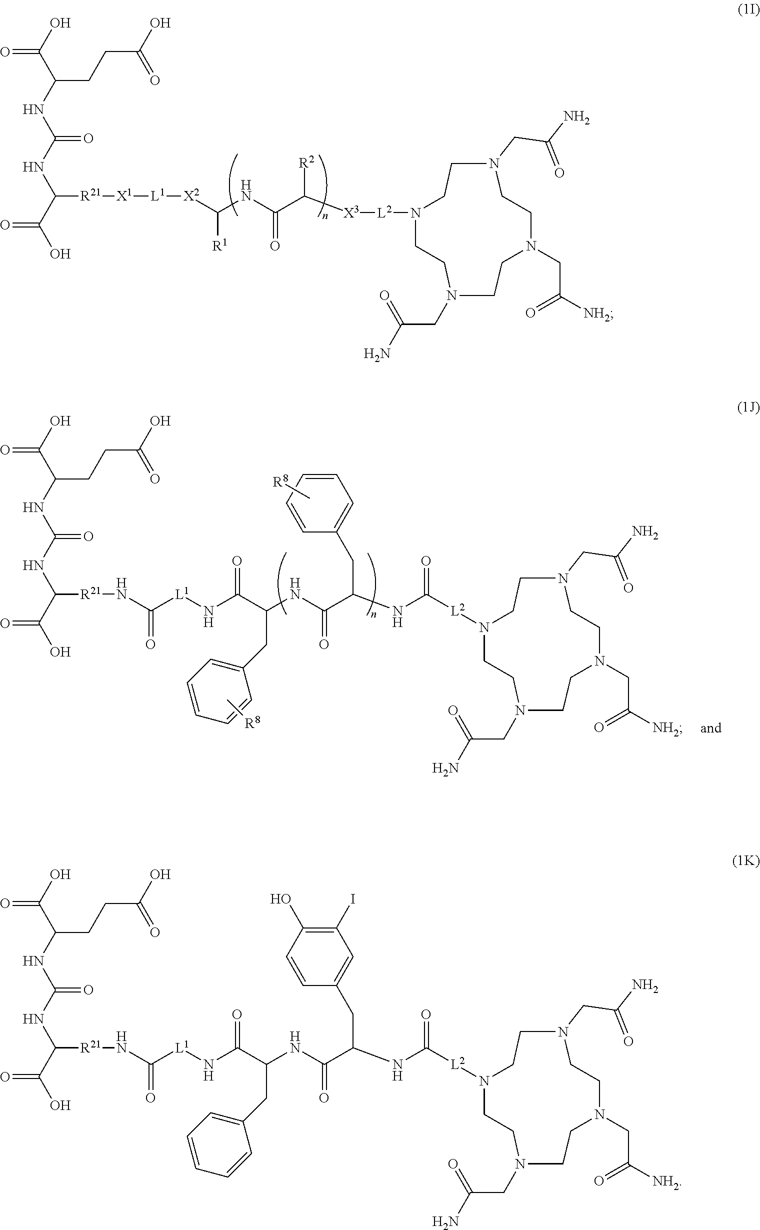

or a pharmaceutically acceptable salt, solvate or stereoisomer thereof;
wherein n, L¹, L², X¹, X², X³, R¹, R², R²¹ and R^{M} are as described herein.

In some embodiments, the compound of Formula (1) has a structure of Formula (1L)

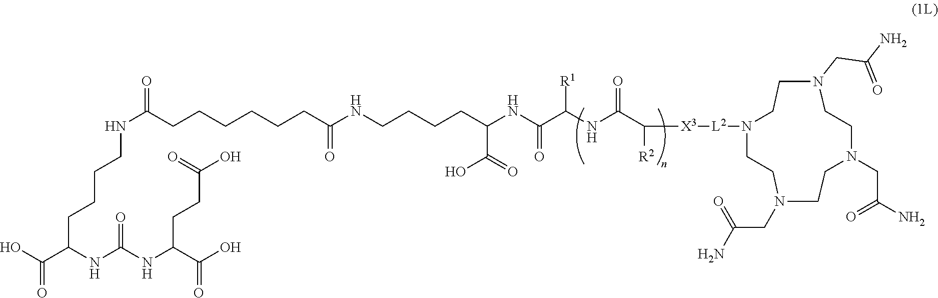

or a pharmaceutically acceptable salt, solvate or stereoisomer thereof;
wherein n, L², X³, R¹, and R² are as described herein.

In one embodiment, the compound of Formula (1) is

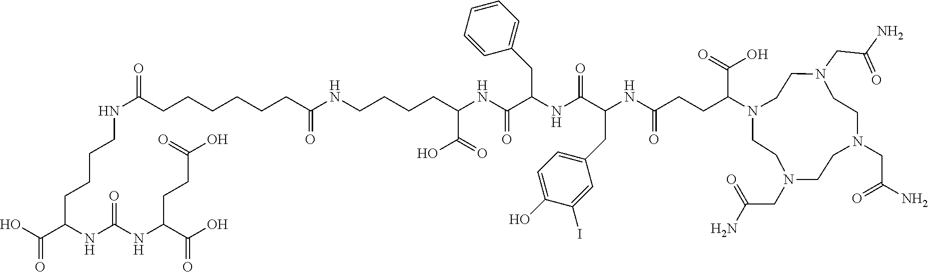 [SMILES: NC(=O)CN1CCN(CC(N)=O)CCN(C(CCC(=O)NC(Cc2ccc(O)c(I)c2)C(=O)NC(Cc2ccccc2)C(=O)NC(CCCCNC(=O)CCCCCCC(=O)NCCCCC(NC(=O)NC(CCC(=O)O)C(=O)O)C(=O)O)C(=O)O)C(=O)O)CCN(CC(N)=O)CC1]

or a pharmaceutically acceptable salt, solvate or stereoisomer thereof.

In some embodiments, the compound of Formula (1) is selected from the group consisting of:

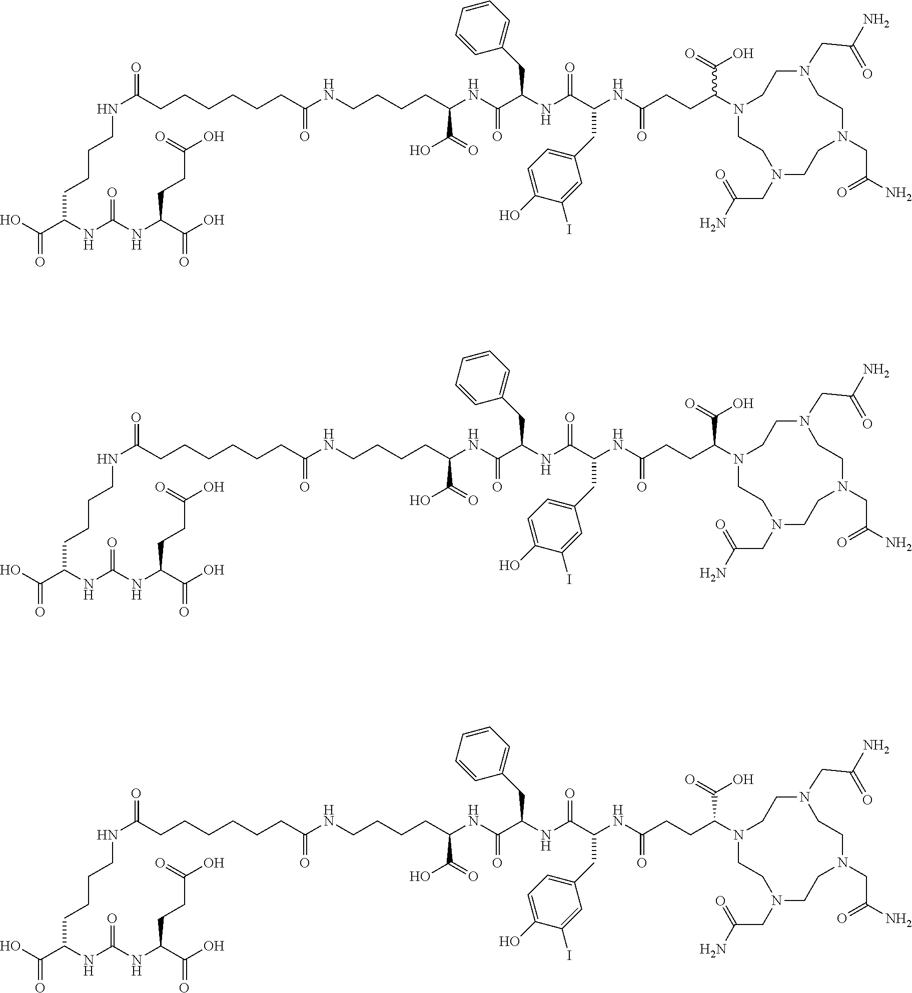

or a pharmaceutically acceptable salt, solvate or stereoisomer thereof.

In some embodiments, the compound of Formula (1) is HO-Glu-CO-Lys[SubA-D-Lys-D-Phe-D-Tyr(3I)-(Pent-²¹²Pb-DO3AM)]-OH (chemical name 3S,7S,26S,29R,32R)-29-benzyl-32-(4-hydroxy-3-iodobenzyl)-5,13,20,28,31,34-hexaoxo-37-(4,7,10-tris(2-amino-2-oxoethyl)-1,4,7,10-tetraazacyclododecan-1-yl)-4,6,12,21,27,30,33-heptaazaheptatriacontane-1,3,7,26,37-pentacarboxylic acid) or a pharmaceutically acceptable salt, solvate or stereoisomer thereof.

In some embodiments, the compound of Formula (1) is:

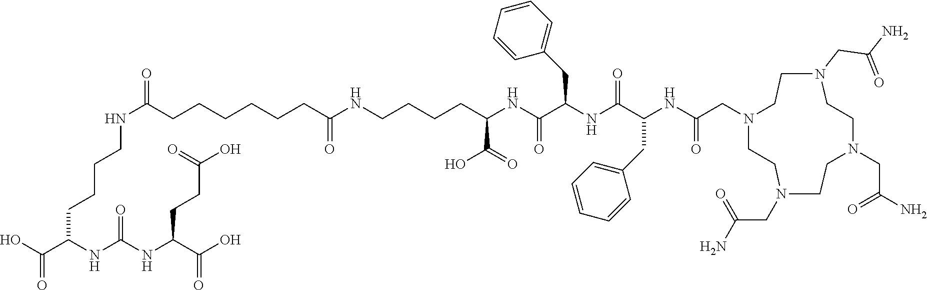 [SMILES: NC(=O)CN1CCN(CC(N)=O)CCN(CC(=O)N[C@H](Cc2ccccc2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)CCN(CC(N)=O)CC1]

or a salt, solvate or stereoisomer thereof.

In some embodiments, the compound of Formula (1) is:

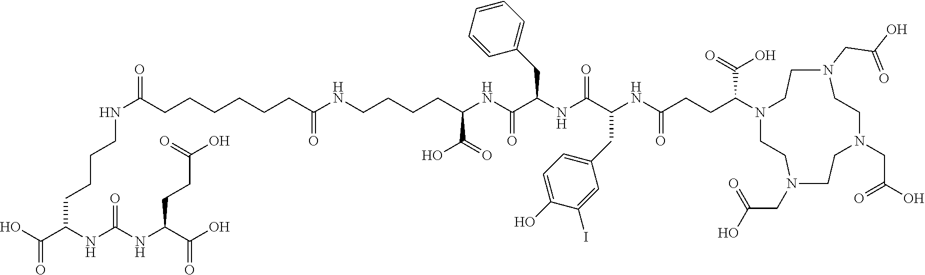 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CC[C@H](C(=O)O)N1CCN(CC(=O)O)CCN(CC(=O)O)CCN(CC(=O)O)CC1)C(=O)O)C(=O)O)C(=O)O]

or a salt, solvate or stereoisomer thereof.

### Indications

While the methods described herein generally relate to certain cancer treatment, such may also be applicable to cardiovascular disease, infection, diabetes, cancer, and/or other conditions.

For cases involving cancer, the cancer may be a cancer that expresses, overexpresses, or upregulates any of the molecular targets or antigens described herein above. For example, in one embodiment the cancer may be a FOLH1-expressing cancer, a SSTR2-overexpressing cancer or a CXCR4-overexpressing cancer.

The cancer may be, for example, a solid tumour derived, for example, either primarily or as a metastatic form, from cancers such as of the liver, prostate, pancreas, head and neck, breast, brain, colon, adenoid, oral, skin, lung, testes, ovaries, cervix, endometrium, bladder, stomach, epithelium, etc.

In some embodiments, the cancer is selected from the group consisting of: carcinoma, sarcoma, leukemia, lymphoma, multiple myeloma, melanoma, central nervous system (CNS) cancers, germ cell tumours, neuroendocrine tumours, mesothelioma, gastrointestinal stromal tumours (GIST), head and neck cancers, thyroid cancer, bladder cancer, kidney cancer, liver cancer, pancreatic cancer, lung cancer, breast cancer, prostate cancer, colorectal cancer, ovarian cancer, cervical cancer, uterine cancer, testicular cancer, esophageal cancer, stomach cancer, gallbladder cancer, bone cancer, soft tissue sarcoma, Ewing sarcoma, rhabdomyosarcoma, wilms tumour, neuroblastoma, retinoblastoma, hodgkin's lymphoma, non-hodgkin's lymphoma, chronic lymphocytic leukemia (CLL), acute lymphoblastic leukemia (ALL), acute myeloid leukemia (AML), chronic myeloid leukemia (CML), myelodysplastic syndromes (MDS), myeloproliferative neoplasms (MPN), non-melanoma skin cancer, basal cell carcinoma, squamous cell carcinoma, thyroid cancer, brain tumours, renal cell carcinoma, glioblastoma, and pancreatic neuroendocrine tumours. In some embodiments, the cancer is metastatic cancer.

In some embodiments, the cancer is prostate cancer. In some embodiments, the cancer is metastatic prostate cancer. In some embodiments, the cancer is metastatic castrate-resistant prostate cancer (mCRPC) or metastatic hormone sensitive prostate cancer (mHSPC). In some embodiments the cancer is metastatic castrate-resistant prostate cancer (mCRPC). In some embodiments the cancer is metastatic hormone sensitive prostate cancer (mHSPC).

In some embodiments, the cancer is a PSMA (prostate-specific membrane antigen) expressing cancer. Prostate-specific membrane antigen (PSMA), also known as folate hydrolase I (FOLH1) and glutamate carboxypeptidase II (GCPII), is a trans-membrane glycoprotein which is primarily expressed in normal human prostate epithelium but which is overexpressed in prostate cancer, including metastatic cancer. Since PSMA is overexpressed in a large proportion of prostate cancers and its expression is further increased in poorly differentiated, metastatic and hormone-refractory carcinomas, it is a very attractive target for PSMA-expressing cancer imaging and therapy.

In some embodiments, the cancer is a SSTR2 (somatostatin receptor 2) expressing cancer. SSTR2 is broadly expressed in meningioma and is a G-protein-coupled receptor, which can be targeted by somatostatin or its synthetic analogs. SSTR2 is widely expressed including in various tumor tissues including neuroendocrine tumors, pituitary adenomas, breast cancer, melanoma, thyroid cancer, and meningioma.

The progress of a cancer is typically determined by assigning a stage to the cancer. Staging is usually carried out by assigning a number from I to IV to the cancer, with I being an isolated cancer and IV being a cancer that has spread to the limit of what the assessment measures. Specifics of staging vary between cancers, but the stage generally takes into account the size of a tumour, whether it has invaded adjacent organs, how many regional (nearby) lymph nodes it has spread to (if any), and whether it has appeared in more distant locations (metastasised).

Generally, Stage I is localised to one part of the body and may be treated by surgical resection (for solid tumours that are small enough). Stage II is locally advanced, and is treatable by chemotherapy, radiation therapy, surgery, or a combination thereof. Stage III is also locally advanced and the designation of Stage II or Stage III depends on the specific type of cancer, although Stage III is generally accepted to be “late” locally advanced. Stage IV cancers have often metastasised to a second organ. Treatment of cancer using a radiopharmaceutical according to the methods of the present disclosure may be used to treat a Stage I, II, III or IV cancer. Treatment according to the methods of the present disclosure may be used to prevent the progression of a cancer from one stage to the next. In one embodiment, treatment according to the methods of the present disclosure is used to prevent progression from Stage I to Stage II. In another embodiment, treatment according to the methods of the present disclosure is used to prevent progression from Stage II to Stage III. In still another embodiment, treatment according to the methods of the present disclosure is used to prevent progression from Stage III to Stage IV.

Preventing or inhibiting progression of the cancer is particularly important for preventing the spread of the cancer, for example the progression from Stage I to Stage II where the cancer spreads locally, or the progression from Stage III to Stage IV where the cancer metastasises to other organs. Methods of treatment according to the present disclosure can therefore be used to prevent the spread of cancer.

In some embodiments, the cancer is relapsed or refractory cancer. For the purposes of the present disclosure, refractory cancers may be taken as cancers that demonstrate resistance to treatment by anti-cancer therapies other than those utilising the methods of the present disclosure. For example, the methods of the present disclosure may be used in the treatment of refractory cancers that are resistant to treatment with radiotherapy (including external beam radiotherapy). Alternatively, or additionally, the methods of the present disclosure may be used in the treatment of refractory cancers that are resistant to biological agents used in the treatment of cancer. In a suitable embodiment, the methods of the present disclosure may be used in the treatment of refractory cancers that are resistant to treatment with methods other than the methods of the present disclosure. Relapsed cancers (or recurrent cancers) are those that return after a period of remission during which the cancer cannot be detected. Cancer recurrence may occur at the site of the original cancer (local cancer recurrence), at a site close to that of the original cancer (regional cancer recurrence), or at a site distant from that of the original cancer (distal cancer recurrence). Accordingly, the methods of the present disclosure may be of great benefit in the context of relapsed cancers. The methods of the present disclosure may be used for the prevention or treatment of a local, regional or distant relapsed cancer.

The methods of the present disclosure may be used to prevent the recurrence of cancer by providing at least about 2, 6, 12, 18, 24, or 30 months of remission. Indeed, the methods of the present disclosure may be used to prevent recurrence of cancer by providing at least about 4, 5, 6, 7, 8, 9, or 10 years of remission.

The methods of the present disclosure may be used to treat a relapsed cancer which has recurred after at least about 2, 6, 12, 18, 24, or 30 months of remission. Indeed, the methods of the present disclosure may be used to treat a relapsed cancer which has recurred after at least about 4, 5, 6, 7, 8, 9, or 10 years of remission.

### Co-Administration

In some embodiments, the radiopharmaceutical may be administered in combination with a further therapeutic agent.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject in need of treatment as disclosed herein. The therapies are administered to a subject in a sequence and within a time interval such that the biological activity or effects overlap. Thus, the term includes sequential as well as coextensive administration of compounds. In a particular embodiment, the therapies are administered to a subject in a sequence and within a time interval such that they provide an increased benefit than if they were administered otherwise. Any additional therapy can be administered in any order with the other additional therapy.

The further therapeutic agent may be any therapeutic agent suitable for co-administration of more than one substance can be for therapeutic and/or prophylactic purposes. If more than one substance or compound is co-administered, the routes of administration of the two or more substances need not be the same. The scope of the methods and uses described herein are not limited by the identity of the substance or substances which may be co-administered with a radiopharmaceutical. Types of fluid that can be co-administered with a radiopharmaceutical should be specific to the circumstances surrounding the particular subject that is suffering from, exhibiting the symptoms of, or at risk of suffering from a bacterial infection. For example, fluids that may be co-administered with a radiopharmaceutical include but are not limited to, electrolytes and/or water, salt solutions, such as sodium chloride and sodium bicarbonate, as well as whole blood, plasma, serum, serum albumin and colloid solutions.

As discussed above, a radiopharmaceutical may for example be administered in combination with one or more additional pharmaceutically active agents. Thus, in some embodiments, the composition comprises a radiopharmaceutical as defined herein, or a pharmaceutically acceptable salt thereof, one or more pharmaceutically acceptable carriers, and one or more additional pharmaceutically active agents.

The present application claims priority from Australian Provisional Patent Application No. 2023902414 filed on 31 Jul. 2023, the contents of which are incorporated herein by reference in their entirety.

### EXAMPLES

### Example 1: General Preclinical Study of the Efficacy of Frequent Dosing With a [²¹²Pb]Pb-Radiopharmaceutical

### Overview

The preclinical study has been designed to test the efficacy of a [²¹²Pb]Pb-radiopharmaceutical in a mouse model of cancer expressing a particular receptor target when administered.

### Method

### Cell Preparation

Human cancer cells expressing a known level of a target of interest are grown up to 80% confluency in cell culture flasks in their respective growth medium. Each culture flask is washed briefly with 10 mL of sterile PBS, pH 7.4 at room temperature. The PBS solution is removed, and the cells are incubated with 10 mL of enzyme-free cell dissociation buffer for 5-15 minutes at 37° C. Once cells appear rounded under the microscope, they are detached from the cell culture flask by gentle pipetting of the dissociation buffer over the cell layer using a 10 mL sterile serological pipette. Cell detachment is confirmed through observation of the cells under a microscope. If detachment is not complete, the flask is gently tapped on the side a few times and incubated at 37° C. for an additional 5 min. Once the detachment is complete, the cell solution is transferred into a sterile falcon tube. The cell solution is centrifuged at 370 rcf for 5 min at room temperature. The cell pellet is resuspended in 1 mL of medium and cells are dissociated by gentle pipetting using a P1000 equipped with 1 mL sterile-filtered tip. The cell solution is diluted up to 4 mL of media. The cell solution concentration is determined by cell-counting in the presence of a live-dead stain (Trypan blue 0.4%), before being centrifuged for 5 min at 370 rcf at room temperature. The supernatant is carefully removed using a P1000 pipette equipped with a sterile-filtered tip and the number of viable cells is determined by the formula: cell count (live cells/ml)×volume of resuspension solution used (4 mL). The cell pellet is resuspended in sterile PBS at a defined concentration allowing tumour uptake.

### Animal Model Preparation

BALB/C nude mice (6-10 weeks old) are acclimatized to the facility for at least 7 days before the experiment. If the chosen cellular model requires Matrigel to establish tumour in nude mice, just prior to cell injection, the cell solution is diluted 1:1 with ice cold Matrigel defrosted overnight on ice. If no Matrigel is required, dilute the cell solution to appropriate concentration using PBS. Animals are placed under anesthesia using isoflurane gas and 100 μl of the cell solution diluted 1:1 in Matrigel (approximately 1 million cells) is injected subcutaneously into the left flank using a 25 G needle. Animals are marked by ear notching for identification and monitored at least three times a week. Monitoring includes general health/behaviour assessment, measurement of animal weight and measurement of tumour volume by digital caliper.

### Animal Treatment

Once tumours reach the desired volume, animals are randomized equally across the predetermined groups for the study (e.g. 8 groups, each with 10 mice). The person skilled in the art will be capable of determining the number of groups for a study, the number of mice per group, as well as the parameters of the dosage regimen that are particular for each group (including regimen dosage (total, and per dosage), the number of doses, and the duration between doses); these may be selected in accordance with standard experimental design, informed by the hypothesis to be tested, and the extent of statistical significance/certainty/power required.

In one example, the radiopharmaceutical may be administered (a) as a single injection (b) as a course of treatment defined by Q9D×2 (c) as a course of treatment defined by Q6D×2 (d) as a course of treatment defined by Q3D×2 (e) as a course of treatment defined by Q9D×3 (f) as a course of treatment defined by Q6D×3, (g) as a course of treatment defined by Q3D×3. The total administered activity is the same for each group (n=7). As noted, the number of groups, size of the groups, MBq dosage, and the number and frequency of the dosages may be modified by the person skilled in the art as required.

| Group | Schedule | Total dose |
|---|---|---|
| 1 (n=10) | 1 μg “cold” radiopharmaceutical | 0.0 MBq |
| 2 (n=10) | 1.5 MBq [²¹²Pb]Pb-radiopharmaceutical; | 1.5 MBq |
| | 1 μg; Single dose | |
| 3 (n=10) | 0.750 MBq [²¹²Pb]Pb-radiopharmaceutical; | 1.5 MBq |
| | 1 μg; Q9Dx2 | |
| 4 (n=10) | 0.750 MBq [²¹²Pb]Pb-radiopharmaceutical; | 1.5 MBq |
| | 1 μg; Q6Dx2 | |
| 5 (n=10) | 0.750 MBq [²¹²Pb]Pb-radiopharmaceutical; | 1.5 MBq |
| | 1 μg; Q3Dx2 | |
| 6 (n=10) | 0.500 MBq [²¹²Pb]Pb-radiopharmaceutical; | 1.5 MBq |
| | 1 μg; Q9Dx3 | |
| 7 (n=10) | 0.500 MBq [²¹²Pb]Pb-radiopharmaceutical; | 1.5 MBq |
| | 1 μg; Q6Dx3 | |
| 8 (n=10) | 0.500 MBq [²¹²Pb]Pb-radiopharmaceutical; | 1.5 MBq |
| | 1 μg; Q3Dx3 | |

The ²¹²Pb labelled radiopharmaceutical (or the “cold” control) is diluted in its respective formulation buffer and injected intravenously via the lateral tail vein using a 0.5 mL insulin syringe. The ligand concentration is kept the same for each treated group.

### Animal Monitoring

Animals are monitored up to three times a week up to 10 days after first treatment. Monitoring may occur for longer, for example up to 2, 3, 4 or more weeks after the first treatment. Monitoring includes a general health/behavioural assessment. Animals are culled once tumour volume reaches a predetermined volume, for example 1000, 1100, 1200, 1300, 1400, or 1500 mm³ or higher, or according to a monitoring scoring sheet.

### Data Analysis

Tumour growth curves may be compared between groups as well as between animal body weight and survival.

### Example 2: Preclinical Study of the Efficacy of Frequent Dosing with a [²¹²Pb]Pb-PSMA-Targeting Radiopharmaceutical

### Overview

A preclinical study was designed in accordance with Example 1, to specifically test the efficacy of the [²¹²Pb]Pb-PSMA-targeting radiopharmaceutical [²¹²Pb]Pb-ADVC001, in a mouse model of PSMA-positive prostate cancer. This study was designed to compare the therapeutic efficacy and toxicity of a dose of the [²¹²Pb]Pb-PSMA-targeting radiopharmaceutical when administered: (a) as a course of treatment defined by Q6D×3, (b) as a course of treatment defined by Q3D×3. The total administered activity was the same for each group (n=2).

### Method

### Cell Preparation

PC3-PIP cells expressing human PSMA were grown up to 80% confluency in cell culture flasks. Each culture flask was washed briefly with 10 mL of sterile PBS, pH 7.4 at room temperature. The PBS solution was removed and the cells incubated with 10 mL of enzyme-free cell dissociation buffer for 5-15 minutes at 37° C. Once cells appeared rounded under the microscope, they were detached from the cell culture flask by gentle pipetting of the dissociation buffer over the cell layer using a 10 mL sterile serological pipette. Cell detachment was confirmed through observation of the cells under a microscope. If detachment was not complete, the flask was gently tapped on the side a few times and incubated at 37° C. for an additional 5 min. Once detachment was complete, the cell solution was transferred into a sterile falcon tube. The cell solution was centrifuged at 370 rcf for 5 min at room temperature. The cell pellet was resuspended in 1 mL of medium and cells dissociated by gentle pipetting using a P1000 equipped with 1 mL sterile-filtered tip. The cell solution was diluted up to 4 mL of media. The cell solution concentration was determined by cell-counting in the presence of a live-dead stain (Trypan blue 0.4%), before being centrifuged for 5 min at 370 rcf at room temperature. The supernatant was carefully removed using a P1000 pipette equipped with a sterile-filtered tip and the number of viable cells determined by the formula: cell count (live cells/ml)×volume of resuspension solution used (4 mL). The cell pellet was resuspended in sterile PBS at a concentration of 20×10⁶ cells/mL.

### Animal Model Preparation

BALB/C nude mice (6-10 weeks old) were acclimatized to the facility for at least 7 days before the experiment. Just prior to cell injection, the cell solution was diluted 1:1 with ice cold Matrigel defrosted overnight on ice. Animals were placed under anesthesia using isoflurane gas, and 100 μl of the cell solution diluted 1:1 in Matrigel (approximately 1 million cells) was injected subcutaneously into the left flank using a 25 G needle. Animals were marked by ear notching for identification and monitored at least three times a week. Monitoring included general health/behaviour assessment, measurement of animal weight and measurement of tumour volume by digital caliper. Once tumours reached ~300 mm³, the animals were randomized into 3 homogenous groups of 10 mice.

### Animal Treatment and Monitoring

The ²¹²Pb labelled radiopharmaceutical (and the “cold” ADVC001 control) was formulated in sodium acetate buffer (0.1 M) with sodium ascorbate (0.25 M) and DTPA (0.1 mg/mL) and injected intravenously via the lateral tail vein using a 0.5 mL insulin syringe. The ligand concentration was kept the same for each treated group.

| Group | Schedule | Total dose |
|---|---|---|
| 1 (n=10) | 1 μg ADVC001 in formulation buffer | 0.0 MBq |
| 2 (n=10) | 0.167 MBq [²¹²Pb]Pb-ADVC001; 1 μg; Q6Dx3 | 0.5 MBq |
| 3 (n=10) | 0.167 MBq [²¹²Pb]Pb-ADVC001; 1 μg; Q3Dx3 | 0.5 MBq |

### Animal Monitoring

Animals were monitored three times a week up to 30 days after the first treatment. Monitoring includes a general health/behavioural assessment. Animals were culled once tumour volume reached 1,500 mm³ or according to monitoring scoring sheet.

### Data Analysis

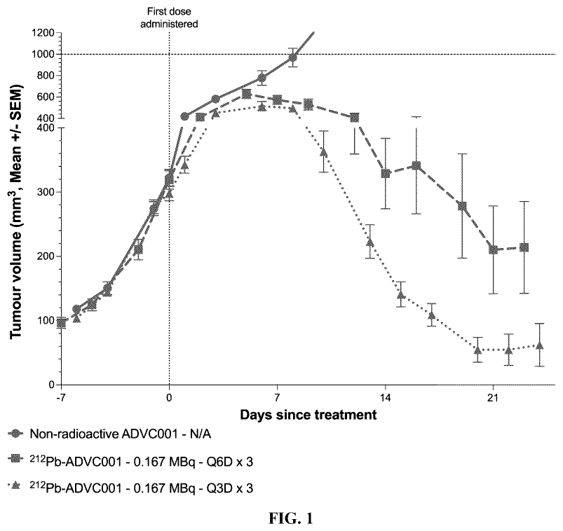

Tumour growth curves were compared between group as well as animal body weight and survival. Tumour growth curves for Groups 1, 2 & 3 are presented in FIG. 1.

### Example 3: Preclinical Study of the Efficacy of Frequent Dosing with a [²¹²Pb]Pb-SSTR2-Targeting Radiopharmaceutical

### Overview

A preclinical study was designed in accordance with Example 1, to specifically test the efficacy of a peptidic [²¹²Pb]Pb-SSTR2-targeting radiopharmaceutical in a mouse model of SSTR2-positive lung cancer. This study was designed to compare the therapeutic efficacy and toxicity of a dose of a [²¹²Pb]Pb-SSTR2-targeting radiopharmaceutical when administered: (a) as a course of treatment defined by Q1W×3 (b) as a course of treatment defined by Q2W×3. The total administered activity is the same for each group (n=10).

### Method

### Cell Preparation

NCI-H69 cells expressing human SSTR2 were grown up to 80% confluency in cell culture flasks. Each culture flask was washed briefly with 10 mL of sterile PBS, pH 7.4 at room temperature. The PBS solution was removed and the cells incubated with 10 mL of enzyme-free cell dissociation buffer for 5-15 minutes at 37° C. Once cells appeared rounded under the microscope, they were detached from the cell culture flask by gentle pipetting of the dissociation buffer over the cell layer using a 10 mL sterile serological pipette. Cell detachment was confirmed through observation of the cells under a microscope. If detachment was not complete, the flask was gently tapped on the side a few times and incubated at 37° C. for an additional 5 min. Once detachment was complete, the cell solution was transferred into a sterile falcon tube. The cell solution was centrifuged at 370 rcf for 5 min at room temperature. The cell pellet was resuspended in 1 mL of medium and cells dissociated by gentle pipetting using a P1000 equipped with 1 mL sterile-filtered tip. The cell solution was diluted up to 4 mL of media. The cell solution concentration was determined by cell-counting in the presence of a live-dead stain (Trypan blue 0.4%), before being centrifuged for 5 min at 370 rcf at room temperature. The supernatant was carefully removed using a P1000 pipette equipped with a sterile-filtered tip and the number of viable cells determined by the formula: cell count (live cells/ml)×volume of resuspension solution used (4 mL). The cell pellet was resuspended in sterile PBS at a concentration of 20×10⁶ cells/mL.

### Animal Model Preparation

BALB/C nude mice (6-10 weeks old) were acclimatized to the facility for at least 7 days before the experiment. Just prior to cell injection, the cell solution was diluted 1:1 with ice cold Matrigel defrosted overnight on ice. Animals were placed under anesthesia using isoflurane gas, and 100 μl of the cell solution diluted 1:1 in Matrigel (approximately 1 million cells) was injected subcutaneously into the left flank using a 25 G needle. Animals were marked by ear notching for identification and monitored at least three times a week. Monitoring includes general health/behaviour assessment, measurement of animal weight and measurement of tumour volume by digital caliper. Once tumours reached about 250 mm³, animals were randomized into 4 homogenous groups of 5-10 mice.

### Animal Treatment

The ²¹²Pb labelled radiopharmaceutical (or “cold” control) was formulated in a sodium acetate buffer and injected intravenously via the lateral tail vein using a 0.5 mL insulin syringe. The ligand concentration was kept the same for each treated group.

| Group | Schedule | Total dose |
|---|---|---|
| 1 (n=10) | 1 μg non-radioactive SSTR2-targeting | 0.00 MBq |
| | compound in formulation buffer | |
| 2 (n=10) | 0.250 MBq [²¹²Pb]Pb-SSTR2-targeting | 0.75 MBq |
| | compound; 1 μg; Q1Wx3 | |
| 3 (n=10) | 0.250 MBq [²¹²Pb]Pb-SSTR2-targeting | 0.75 MBq |
| | compound; 1 μg; Q2Wx3 | |

### Animal Monitoring

Animals were monitored three times a week up to 30 days after first treatment. Monitoring includes a general health/behavioural assessment. Animals were culled once tumour volume reaches 1,000 mm³ or according to monitoring scoring sheet.

### Data Analysis

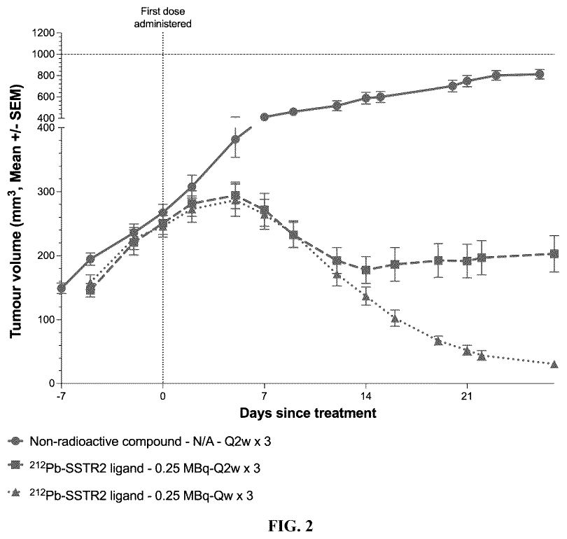

Tumour growth curves were compared between group as well as animal body weight and survival. Tumour growth curves for Groups 1, 2, 3 and 4 are presented in FIG. 2.

### Example 4: Clinical Trial Design and Dosing Schedule

The clinical trial design is a prospective, Phase Ib, single arm, non-randomised, dose escalation and dose interval study designed to explore safety, tolerability and therapeutic efficacy of a radiopharmaceutical comprising a therapeutic isotope having a half-life of less than about 24 hours, such as a, [²¹²Pb]Pb-radiopharmaceutical, such as [²¹²Pb]Pb-PSMA-targeting radiopharmaceutical, such as [²¹²Pb]Pb-ADVC001, administered with varying compressed dosing intervals of 4 weeks or less to subjects with metastatic castration-resistant PSMA-expressing prostate cancer (mCRPC).

### Study Objectives

### Primary Objective

To determine the recommended phase 2 dose (RP2D), including administered activity and dosing frequency, of a [²¹²Pb]Pb-PSMA-targeting radiopharmaceutical.

### Secondary Objective

To evaluate the safety and tolerability of a [²¹²Pb]Pb-PSMA-targeting radiopharmaceutical administered at dosing intervals of 4 weeks or less as described below in patients with metastatic castration-resistant PSMA-expressing prostate cancer.

### Study Design and Duration

The planned study duration is approximately 48 months. The study duration for an individual participant is approximately up to 36 months, comprised of the following study periods:
Screening Period: Up to 28 days prior to Day 1;
Treatment Period: Up to 24 weeks, consisting of up to 6 doses administered 2 or 4 weeks apart;
Follow-up Period: Up to 36 months following Cycle 1 Day 1.

### Treatment Cohort and Administration

Each participant will receive up to 6 doses of a [²¹²Pb]Pb-PSMA-targeting radiopharmaceutical administered at approximately 2- or 4-week intervals (each dose representing a treatment cycle). The [²¹²Pb]Pb-PSMA-targeting radiopharmaceutical will be administered on Day 1 of each treatment cycle by slow (over 2 to 10 minutes) intravenous (IV) injection.

Participants will be enrolled into one of six dose interval cohorts
Cohort 1a: 120 MBq per dose of the [²¹²Pb]Pb-PSMA-targeting radiopharmaceutical administered at 4-week (+/−1 week) intervals
Cohort 1b: 120 MBq per dose of the [²¹²Pb]Pb-PSMA-targeting radiopharmaceutical administered at 2-week (+/−1 week) intervals
Cohort 2a: 160 MBq per dose of the [²¹²Pb]Pb-PSMA-targeting radiopharmaceutical administered at 4-week (+/−1 week) intervals
Cohort 2b: 160 MBq per dose of the [²¹²Pb]Pb-PSMA-targeting radiopharmaceutical administered at 2-week (+/−1 week) intervals
Cohort 3a: 200 MBq per dose of the [²¹²Pb]Pb-PSMA-targeting radiopharmaceutical administered at 4-week (+/−1 week) intervals
Cohort 3b: 200 MBq per dose of the [²¹²Pb]Pb-PSMA-targeting radiopharmaceutical administered at 2-week (+/−1 week) intervals

Each cohort may backfill with additional participants for further characterization of safety, tolerability, and preliminary efficacy of doses and schedules, including additional dose schedules at +1-week of proposed intervals, at or below the maximum tolerated dose (MTD).

### Eligibility Criteria

The eligibility criteria of the study can include one or more of the following: Male participant, aged 18 years or older at the time of consent.

Has documented metastatic adenocarcinoma of the prostate, confirmed by histopathology.

Has metastatic disease (≥1 metastatic lesion present on baseline computed tomography [CT], magnetic resonance imaging [MRI] or bone scintigraphy).

Has castration-resistant prostate cancer progressing or has progressed on androgen receptor therapy and must have castrate level of serum/plasma testosterone (≤50 ng/dl or ≤1.7 nmol/L).

Has had exposure to a taxane-based chemotherapy at any time in the course of their disease (unless taxanes considered contraindicated or declined by patient).

Has disease that is progressing at Screening, despite a castrate testosterone level (≤50 ng/dL or ≤1.7 nmol/L) by the demonstration of at least one of the following:
a) Increase in PSA greater than 25% and >2 ng/mL above nadir, confirmed by progression at two timepoints at least 3 weeks apart;
b) Progressive disease or new lesion(s) (relative to previous imaging) in the viscera or lymph nodes as per RECIST 1.1 or in bone as per Prostate Cancer Working Group 3 (PCWG3). Any ambiguous results are to be confirmed by additional imaging modality (e.g., CT or MRI, ^{99m}Tc bone scintigraphy).

Has disease that is prostate specific membrane antigen (PSMA) positive, as demonstrated by ⁶⁸Ga-PSMA-PET/CT or ¹⁸F-based PSMA PET/CT and confirmed as eligible by local reader. PSMA-positive participants are defined as those having at least one tumour lesion with ⁶⁸Ga or ¹⁸F PSMA PET CT uptake greater than normal liver (based on visual assessment) and all tumour lesions larger than size criteria with ⁶⁸Ga or ¹⁸F-PSMA uptake greater than liver [short axis size criteria: organs ≥1 cm, lymph nodes ≥2.5 cm, bones (soft tissue component) ≥1 cm].

Be Eastern Co-operative Oncology Group (ECOG) Performance Status 0 or 1.

Has adequate renal, haematological, and liver function, as defined by one or more of the following safety laboratory results at Screening:
Absolute neutrophil count: ≥2×10⁹/L;
Hemoglobin: ≥90 g/L;
Platelet count: >150,000×10⁹/L;
Serum creatinine: <1.5× upper limit of normal (ULN) i.e ≤125 μmol/L or calculated creatinine clearance ≥60 mL/min/1.73 m² by Cockcroft-Gault formula;
Serum total bilirubin: <1.5×ULN (unless the patient has Gilbert's syndrome in which case direct bilirubin must be normal);
Serum aspartate aminotransferase (AST) and alanine transaminase (ALT): <1.5× ULN in the absence of liver metastases; <3× ULN if due to liver metastases (in both circumstances bilirubin must meet entry criteria).

Has the capacity to understand the study and be willing and able to comply with all study requirements, including the timing and nature of all required assessments.

Must agree to comply with the radiation protection guidelines (including hospital admissions and isolation, where relevant) that are applied by the treating institution.

Must agree to practice adequate precautions to prevent pregnancy in a partner to avoid potential problems associated with radiation exposure to the unborn child.

Estimated life expectancy >12 weeks.

### Study Endpoints

The end point of the study can include one or more of the following: Primary Endpoints:

Incidence and severity of dose-limiting toxicities assessed in accordance with NCI CTCAE Version 5.0.

### Secondary Endpoints:

PSA response defined as a reduction from baseline PSA level of at least 50% (PSA 50) and 90% (PSA 90), confirmed by a follow-up PSA result.

Objective response rate (ORR) and disease control rate (DCR) in participants with measurable disease (RECIST 1.1).

Radiographic PFS (rPFS) defined as the time from date of first dosing to the occurrence of one of the following:
Progression of measurable lesions using RECIST 1.1;
Progression of bone lesions using PCWG3 criteria;
Death due to any cause

### Overall Survival

Absorbed radiation doses (expressed as Gy/MBq) of administered the [²¹²Pb]Pb-PSMA targeting radiopharmaceutical to organs at risk and tumour lesions.

### Example 5: 212Pb-PSMA Targeted SPECT/CT Imaging in a Patent with Metastatic Castration-Resistant Prostate Cancer

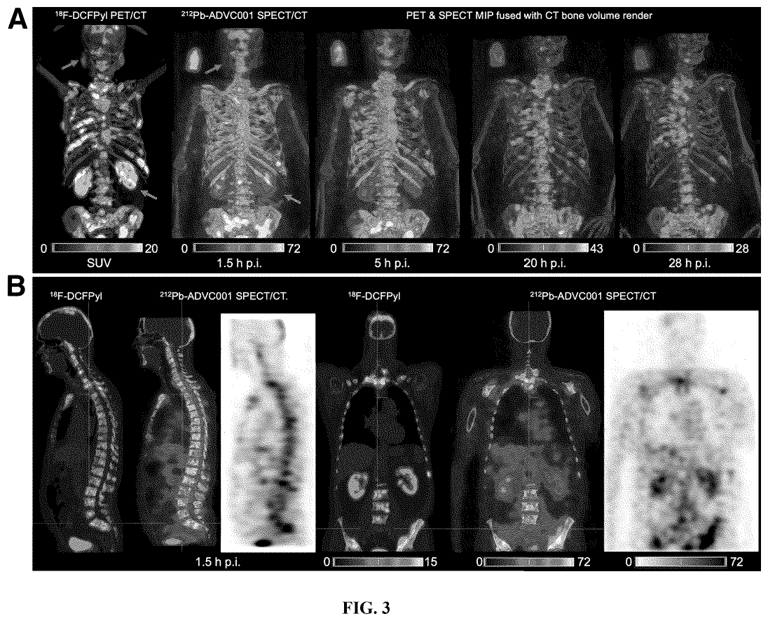

To gauge the biodistribution and inform patient tolerability of a ²¹²Pb radiopharmaceutical comprising an acetamide-based chelator, 60 MBq of ²¹²Pb-ADVC001 was administered to a 73 year old man with metastatic castration-resistant prostate cancer. SPECT/CT imaging was performed at 1.5, 5, 20, and 28 h following infusion. Two simultaneous triple-energy window acquisitions (78 keV±20% with 20% scatter [31% abundance] and 239 keV±10% with 10% scatter [43% abundance] were obtained using a Siemens Intevo Bold (high-energy collimators at 30 s per view for 120 views per rotation at 2 bed positions with noncircular orbits; total time, 60 min). Each energy window was reconstructed independently, and the resulting images were summed with removal of Compton-based orbit artifacts. Representative ²¹²Pb SPECT/CT images (FIG. 3) showed rapid tumour uptake of ²¹²Pb-ADVC001 highly concordant with tumour burden delineated on the pretreatment ¹⁸F-DCFPyl PET/CT images. Images acquired after 20 h showed persistent tumour uptake despite low counts due to ²¹²Pb decay (10.6 h half-life). Depicted in FIG. 3A, ¹⁸F-DCFPyl PET/CT and ²¹²Pb SPECT/CT images showed concordant tumour biodistribution with low salivary gland uptake (indicated with arrows) and rapid kidney clearance of 60 MBq of ²¹²Pb-ADVC001, which is indicative of patient tolerability of ²¹²Pb-ADVC001 in dosage regimens with dosing frequencies less than 4 weeks. FIG. 3B depicts sagittal and coronal images at 1.5 h after injection (p.i.). Further details are published in Griffths et al., Journal of Nuclear Medicine, 2024, 65 (7), the entire contents of which are hereby incorporated herein by reference.

1. A method of treating a human subject with metastatic prostate cancer with bone metastases comprising:
administering to the human subject a therapeutically effective amount of a radiopharmaceutical therapy composition comprising a prostate-specific membrane antigen (PSMA) targeting ligand having ²¹²Pb chelated thereto, wherein the radiopharmaceutical therapy composition is administered to the human subject in at least two treatment cycles separated by an interval of 7 to 14 days and at an activity per dose of between about 60 MBq to 500 MBq and wherein the metastatic prostate cancer with bone metastases is treated.

2. The method of claim 1, wherein the metastatic prostate cancer is metastatic castration-resistant prostate cancer.

3. The method of claim 1, wherein the human subject has previously received at least one androgen receptor pathway inhibitor.

4. The method of claim 1, wherein the human subject is monitored for progression of bone lesions.

5. The method of claim 1, wherein the human subject remains in progression free survival for at least a period of 6 months.

6. The method of claim 1, wherein the PSMA-targeting ligand has a structure of: 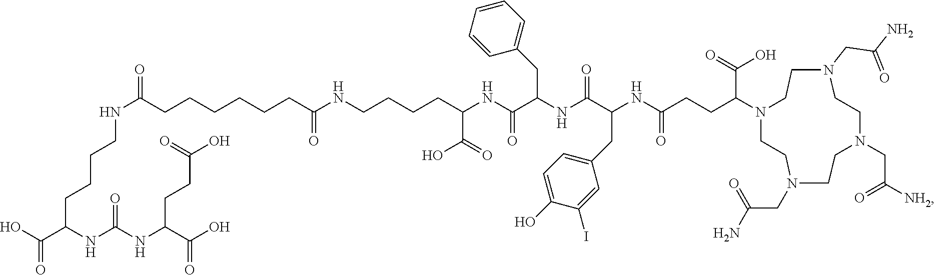 [SMILES: NC(=O)CN1CCN(CC(N)=O)CCN(C(CCC(=O)NC(Cc2ccc(O)c(I)c2)C(=O)NC(Cc2ccccc2)C(=O)NC(CCCCNC(=O)CCCCCCC(=O)NCCCCC(NC(=O)NC(CCC(=O)O)C(=O)O)C(=O)O)C(=O)O)C(=O)O)CCN(CC(N)=O)CC1]
or a pharmaceutically acceptable salt, solvate or stereoisomer thereof.

7. The method of claim 1, wherein the PSMA-targeting ligand has a structure of: 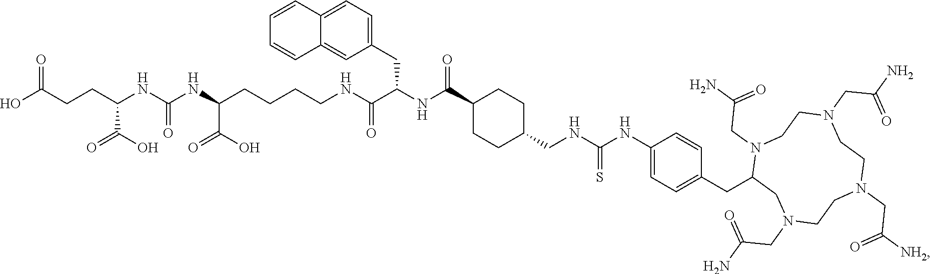 [SMILES: NC(=O)CN1CCN(CC(N)=O)CCN(CC(N)=O)C(Cc2ccc(NC(=S)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)cc2)CN(CC(N)=O)CC1]
or a pharmaceutically acceptable salt, solvate or stereoisomer thereof.

8. The method of claim 1, wherein the human subject exhibits a criteria selected from histologically confirmed adenocarcinoma of the prostate, cytologically confirmed adenocarcinoma of the prostate, demonstrated PSMA-positive disease as determined by PSMA-targeted positron emission tomography (PET) imaging, wherein at least one metastatic lesion exhibits radiotracer uptake greater than uptake in normal liver tissue, radiographically documented metastatic disease comprising at least one bone metastatic lesion, and an Eastern Cooperative Oncology Group (ECOG) performance status of 0, 1 or 2.

9. A method of treating advanced prostate cancer in a human subject in need thereof comprising:
intravenously administering to the human subject a therapeutically effective amount of a radiopharmaceutical comprising a prostate-specific membrane antigen (PSMA) targeting ligand chelated to ²¹²Pb;
wherein the human subject has at least one metastatic lesion detected by computed tomography, magnetic resonance imaging, bone scintigraphy, or any combination thereof;
wherein the therapeutically effective amount of the radiopharmaceutical has an activity per dose of about 1 MBq to about 600 MBq and is administered to the human subject in at least two treatment cycles separated by an interval of one to four weeks;
wherein the advanced prostate cancer is treated.

10. The method of claim 9, wherein the human subject has at least one metastatic lesion detected by bone scintigraphy.

11. The method of claim 9, wherein the metastatic prostate cancer is metastatic castration-resistant prostate cancer.

12. The method of claim 9, wherein the human subject is monitored for progression of bone lesions using prostate cancer clinical trials working group 3 criteria.

13. The method of claim 9, wherein the human subject remains in progression free survival for at least a period of 6 months.

14. The method of claim 9, wherein the PSMA-targeting ligand has a structure of: 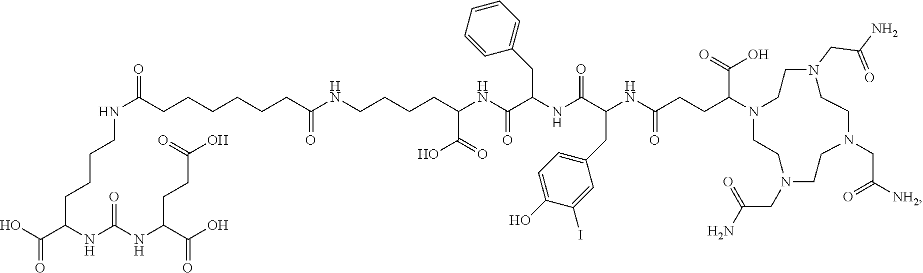 [SMILES: NC(=O)CN1CCN(CC(N)=O)CCN(C(CCC(=O)NC(Cc2ccc(O)c(I)c2)C(=O)NC(Cc2ccccc2)C(=O)NC(CCCCNC(=O)CCCCCCC(=O)NCCCCC(NC(=O)NC(CCC(=O)O)C(=O)O)C(=O)O)C(=O)O)C(=O)O)CCN(CC(N)=O)CC1]
or a pharmaceutically acceptable salt, solvate or stereoisomer thereof.

15. The method of claim 9, wherein the PSMA-targeting ligand has a structure of: 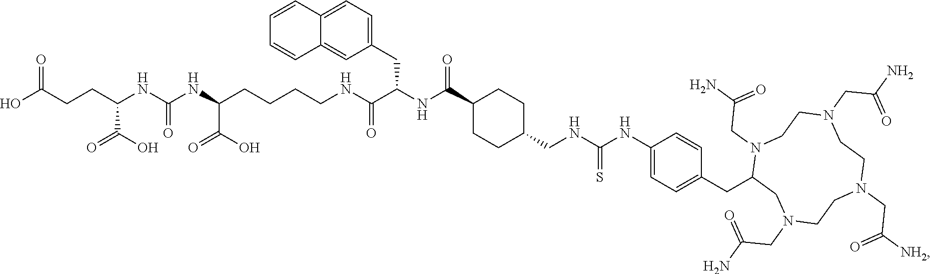 [SMILES: NC(=O)CN1CCN(CC(N)=O)CCN(CC(N)=O)C(Cc2ccc(NC(=S)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)cc2)CN(CC(N)=O)CC1]
or a pharmaceutically acceptable salt, solvate or stereoisomer thereof.

16. The method of claim 9, wherein treatment results in no progression of bone lesions.

17. The method of claim 9, wherein the therapeutically effective amount of the radiopharmaceutical has an activity per dose of about 60 MBq to about 500 MBq.

18. The method of claim 9, wherein the radiopharmaceutical is administered to the human subject in at least two treatment cycles separated by an interval of one to two weeks.

19. A method of inhibiting the progression of metastatic prostate cancer in a human subject with a bone lesion comprising:
intravenously administering to the human subject a therapeutically effective amount of a radiopharmaceutical therapy composition comprising a prostate-specific membrane antigen (PSMA) targeting ligand having ²¹²Pb chelated thereto, wherein the radiopharmaceutical therapy composition is administered to the human subject in at least two treatment cycles separated by an interval of 7 to 14 days and at an activity per dose of between about 60 MBq to 500 MBq and wherein the metastatic prostate is treated.

20. The method of claim 19, wherein the metastatic prostate cancer is metastatic castration-resistant prostate cancer.

21. The method of claim 19, wherein the human subject remains in progression free survival for at least a period of 6 months.

22. The method of claim 19, wherein the wherein the PSMA-targeting ligand has a structure of: 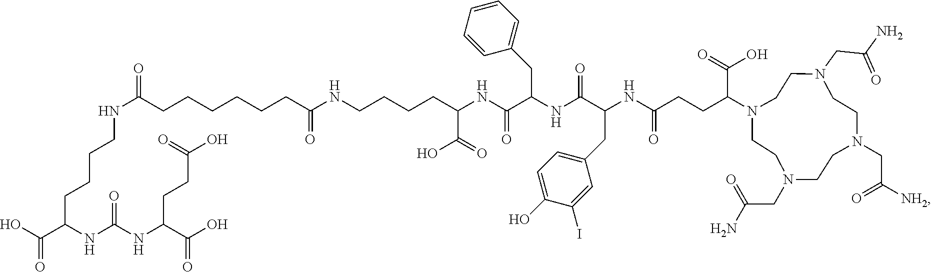 [SMILES: NC(=O)CN1CCN(CC(N)=O)CCN(C(CCC(=O)NC(Cc2ccc(O)c(I)c2)C(=O)NC(Cc2ccccc2)C(=O)NC(CCCCNC(=O)CCCCCCC(=O)NCCCCC(NC(=O)NC(CCC(=O)O)C(=O)O)C(=O)O)C(=O)O)C(=O)O)CCN(CC(N)=O)CC1]
or a pharmaceutically acceptable salt, solvate or stereoisomer thereof.

23. The method of claim 19, wherein the PSMA-targeting ligand has a structure of: 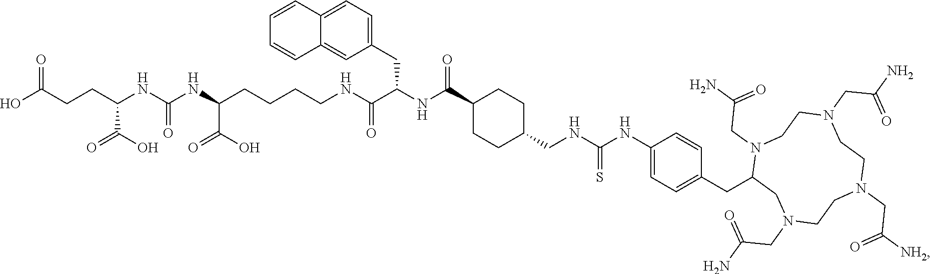 [SMILES: NC(=O)CN1CCN(CC(N)=O)CCN(CC(N)=O)C(Cc2ccc(NC(=S)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)cc2)CN(CC(N)=O)CC1]
or a pharmaceutically acceptable salt, solvate or stereoisomer thereof.

24. The method of claim 19, wherein treatment results in no progression of bone lesions.

25. The method of claim 19, wherein the therapeutically effective amount of the radiopharmaceutical has an activity per dose of about 60 MBq to about 500 MBq.

26. The method of claim 19, wherein the radiopharmaceutical therapy composition is administered to the human subject in at least two treatment cycles separated by an interval of one to two weeks.

27. The method of claim 1, wherein the therapeutically effective amount of the radiopharmaceutical therapy composition is administered intravenously.

28. A method of treating a human subject with metastatic prostate cancer with bone metastases comprising:
administering to the human subject a therapeutically effective amount of a radiopharmaceutical therapy composition comprising a prostate-specific membrane antigen (PSMA) targeting ligand having ²¹²Pb chelated thereto,
wherein the human subject exhibits a criteria selected from histologically confirmed adenocarcinoma of the prostate, cytologically confirmed adenocarcinoma of the prostate, demonstrated PSMA-positive disease as determined by PSMA-targeted positron emission tomography (PET) imaging, wherein at least one metastatic lesion exhibits radiotracer uptake greater than uptake in normal liver tissue, radiographically documented metastatic disease comprising at least one bone metastatic lesion, and an Eastern Cooperative Oncology Group (ECOG) performance status of 0, 1 or 2;
wherein the radiopharmaceutical therapy composition is administered to the human subject in at least two treatment cycles separated by an interval of 7 to 14 days and at an activity per dose of between about 60 MBq to 500 MBq; and
wherein the metastatic prostate cancer with bone metastases is treated, wherein treatment is progression free survival for at least 6 months.

29. The method of claim 28, wherein the PSMA-targeting ligand has a structure of: 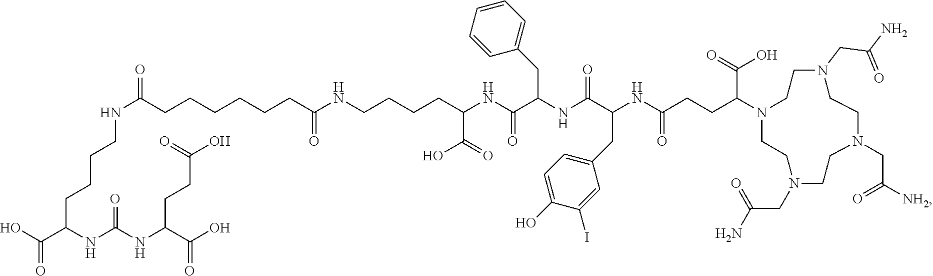 [SMILES: NC(=O)CN1CCN(CC(N)=O)CCN(C(CCC(=O)NC(Cc2ccc(O)c(I)c2)C(=O)NC(Cc2ccccc2)C(=O)NC(CCCCNC(=O)CCCCCCC(=O)NCCCCC(NC(=O)NC(CCC(=O)O)C(=O)O)C(=O)O)C(=O)O)C(=O)O)CCN(CC(N)=O)CC1]
or a pharmaceutically acceptable salt, solvate or stereoisomer thereof.

30. The method of claim 29, wherein the PSMA-targeting ligand has a structure of: 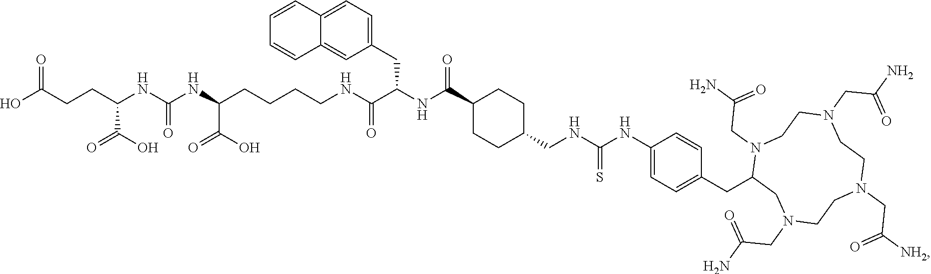 [SMILES: NC(=O)CN1CCN(CC(N)=O)CCN(CC(N)=O)C(Cc2ccc(NC(=S)NC[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)cc2)CN(CC(N)=O)CC1]
or a pharmaceutically acceptable salt, solvate or stereoisomer thereof.
